(19) United States
(12) Patent Application Publication
(10) Pub. No.: US 2023/0295256 A1
(43) Pub. Date: Sep. 21, 2023

(54) COMPOSITIONS FROM GASTROINTESTINAL TRACT MUCINS, AND USES THEREOF

(71) Applicant: Gnubiotics Sciences SA, Epalinges (CH)

(72) Inventors: Adeyemi Adesokan, Epalinges (CH); Yong Miao, Epalinges (CH); Sara Vidal López, Spiez (CH)

(21) Appl. No.: 18/195,078

(22) Filed: May 9, 2023

Related U.S. Application Data
(63) Continuation of application No. 16/687,665, filed on Nov. 18, 2019.
(60) Provisional application No. 62/769,555, filed on Nov. 19, 2018.
(60) Provisional application No. 62/831,627, filed on Apr. 9, 2019.
(60) Provisional application No. 62/880,630, filed on Jul. 30, 2019.
(60) Provisional application No. 62/888,436, filed on Aug. 16, 2019.

(51) Int. Cl. C07K 14/47; A23K 20/147; A23K 20/163; A61K 9/00; C07K 1/34

(52) U.S. Cl. CPC C07K 14/4727; A23K 20/147; A23K 20/163; A61K 9/0053; C07K 1/34; A61K 38/00

(57) ABSTRACT

Disclosed are compositions comprising glycopeptides obtained from gastrointestinal mucins that have superior microbiota affects, and methods of manufacture and use thereof. Such compositions are advantageous for pharmaceutical, food stuff and pet food applications.

### RELATED APPLICATIONS

This application is a continuation of U.S. application Ser. No. 16/687,665 filed Nov. 18, 2019, which claims the benefit of U.S. Provisional Application Ser. No. 62/769,555, filed Nov. 19, 2018, U.S. Provisional Application Ser. No. 62/831,627, filed Apr. 9, 2019, U.S. Provisional Application Ser. No. 62/880,630, filed Jul. 30, 2019, and U.S. Provisional Application Ser. No. 62/888,436, filed Aug. 16, 2019; the contents of all of which are hereby incorporated by reference in their entirety.

### FIELD OF THE INVENTION

The present invention pertains generally to the fields of compositions containing glycopeptides and products derived therefrom, in particular compositions useful as nutritional supplements, such as medical nutrition, domestic animal nutrition, and nutraceutical products that enhance the growth of beneficial microorganisms in the mammalian microbiome, such as Akkermansia muciniphila, and promote production of short-chain fatty acids (SCFA) in the gut. In some embodiments, the present invention pertains to animal feed comprising compositions containing glycopeptides.

### BACKGROUND OF THE INVENTION

Hydrolyzed animal mucosa is a waste product produced during industrial processes. Highly purified forms of this waste product have been used as a protein additive in animal feed having nutritional and physiological benefits such as faster growth, enhanced feed utilization, and improved palatability.

It has also been recently recognized that the dense microbial community (microbiota) present in the mammalian, and in particular human, intestine shortly after birth and throughout the life has a profound effect on health and physiology.

One major factor shaping the composition and physiology of the microbiota is the influx of glycans into the intestine, mostly from diet and host mucosal secretions. Humans consume dozens of different plant- and animal-derived dietary glycans, most of which cannot be degraded by enzymes encoded in the human genome. Microbial fermentation transforms these indigestible glycans into short chain fatty acids which serve as nutrients for colonocytes and other gut epithelial cells (i.e., intestinal epithelial cells). Gut microorganisms therefore play a pivotal symbiotic role in helping mammals (e.g., humans, dogs, cats, and livestock) access calories from otherwise indigestible nutrients and each type of microorganisms prefer different glycans. Therefore, a selective consumption of nutrients can influence which microbial groups proliferate and persist in the gastrointestinal tract. Dietary glycans have been considered as being a possible non-invasive strategy of directly influencing the balance of bacterial species in the gut (Koropathkin et al., 2012, Nat Rev Microbiol. 10(5):323-35).

Gut microbes play an important role in the regulation of host metabolism and low-grade inflammation. Abnormalities in microbiota composition and activity (called dysbiosis) have been implicated in the emergence of the metabolic syndrome, which include diseases such as obesity, type 2 diabetes and cardiovascular diseases. One of the bacteria that influence human metabolism and is found in infant and adult intestinal track (0.5-5% of the total bacteria) as well as in human milk is Akkermansia muciniphila (Derrien et al., 2008, Appl Environ Microbiol., 74(5): 1646-1648; Cani et al., 2017, Front Microbiol., 8: 1765).

Akkermansia muciniphila is a Gram-negative, anaerobic, non-spore-forming bacterium, within genus Akkermansia, from the family—Verrucomicrobiaceae, which is the most abundant mucus degrading bacterium in the healthy individual. The host and Akkermansia communicate continually and this interaction creates a positive feedback loop in which Akkermansia degrades the mucus layer which stimulates new mucus production and the production of new mucus stimulates growth of Akkermansia. This process ensures that abundant amounts of Akkermansia maintain the integrity and shape of the mucus layer. Akkermansia produces important metabolites as a result of the mucus degradation process, in particular two very important short chain fatty acids (SCFA): acetic acid and propionic acid, which trigger a cascade of responses in the host having a crucial role in immune stimulation and metabolic signaling (Derrien et al., 2011, Front Microbiol., 2: 166).

Recent evidence demonstrates that gut concentration of Akkermansia muciniphila is inversely associated with obesity, diabetes, cardiometabolic diseases and low-grade inflammation. Therefore, this bacterium is considered a potential candidate for improving the conditions of subjects suffering or at risk of suffering from those disorders (Cani et al., 2017, supra).

In particular, Akkermansia muciniphila's numbers were higher in pregnant women with normal weight gain than in those with excessive weight gain (Santacruz et al., 2010, Br J Nutr., 104(1):83-92) and Akkermansia muciniphila-like bacteria were significantly lower in the obese/overweight pre-school children (Karlsson et al., 2012, Obesity, 20(11): 2257-61). Akkermansia muciniphila was also shown to inversely correlate with the onset of inflammation, altered adipose tissue metabolism and metabolic disorders during obesity in mice (Schneeberger et al., 2015, Scientific Reports, 5: 16643) and was shown to improve metabolic health during a dietary intervention (calorie restriction) in overweight/obese adults (Dao et al., 2016, Gut, 65(3): 426-36). Akkermansia muciniphila was also shown to be inversely related to the severity of the acute appendicitis (Swidsinski et al., 2011, Gut, 60(1):34-40) and was suggested to play a protective role in autoimmune diabetes development, particularly during infancy (Hansen et al., 2012, Diabetologia, 55(8):2285-94). Further, and not least, a correlation between clinical responses to immune checkpoint inhibitors (ICIs) targeting the PD-1/PD-L1 axis (Programmed cell death protein 1/Programmed death-ligand 1) in cancer patients (non-small cell lung carcinoma, renal cell carcinoma) and the relative abundance of Akkermansia muciniphila was found. In particular, it was shown that fecal microbiota transplantation (FMT) from cancer patients who responded to ICIs into germ-free or antibiotic-treated mice ameliorated the antitumor effects of PD-1 blockade (Routy et al., 2017, Science, 359(6371):91-97).

A possibility that has been investigated to enhance the population of Akkermansia muciniphila in the gut is the administration of live or pasteurized Akkermansia muciniphila in the form of oral supplementation. There is an issue, however, of preserving the viability of Akkermansia muciniphila during production and storage prior to administration of those supplements (Cani et al., 2017, supra). No commercially available probiotic supplement currently exists that contains Akkermansia muciniphila. Alternatively, increasing Akkermansia muciniphila can be achieved through the consumption of certain prebiotics and polyphenol-rich foods. However, the efficacy of those prebiotics and polyphenol-rich foods is limited.

Besides Akkermansia muciniphila, other commensal bacteria including Megamonas spp., Coprococcus comes, and Bacteroides spp. are also known producers of SCFA in the gut. Thus, a prebiotic that can increase the population of these bacteria would be advantageous for improving host health.

### SUMMARY OF THE INVENTION

The present invention pertains to the surprising discovery that compositions obtained from gastrointestinal tract mucins, under conditions wherein the mucins or a partially purified fraction thereof are not subject to conditions or reagents that release oligosaccharides from glycoproteins or glycopeptides, promote beneficial bacteria growth in the gut including growth of Bifidobacterium bifidum, Bifidobacterium animalis subsp. lactis, Lactobacillus acidophilus, Bifidobacterium breve, Bacteroides thetaiotaomicron, Megamonas spp., Prevotella copri, Bacteroides vulgatus, Coprococcus comes, and Akkermansia muciniphila. Furthermore, the inventors surprisingly found that such compositions, unlike unprocessed mucin samples, do not promote excessive Escherichia coli growth. Such compositions, which comprise oligosaccharides bound to glycopeptides, are also better utilized by beneficial bacteria than free oligosaccharides (i.e., free glycans). See, e.g., WO 2019049157, incorporated herein by reference.

Without being bound by theory, the present inventors also believe that the compositions provided herein promote the extended growth of beneficial bacteria in the gut possibly because the bound glycans are depleted more slowly by gut bacteria than free glycans. Specifically, although prior art such as U.S. Pat. No. 8,795,746 (issued Aug. 5, 2018) teach cleaving glycans from amino acid backbones, and using compositions enriched in cleaved glycans to promote gut bacteria, the inventors have surprisingly found that compositions comprising glycoproteins and glycopeptides and not enriched in cleaved glycans promote superior bacterial growth in the gut, including growth of Akkermansia muciniphila.

Applicants have surprisingly found that a good source of glycopeptides for obtaining the compositions of the claimed invention are partially purified gastrointestinal tract mucins produced as a waste product in other industrial processes. Using such waste stream products as a starting material can significantly reduce the manufacturing cost of the present compositions, a clear advantage when utilizing the compositions in commercial products.

Some aspects of the invention are directed to a composition comprising a mixture of glycopeptides obtained from gastrointestinal tract mucins, wherein the composition is obtained without subjecting the mucins or a partially purified fraction thereof to conditions or reagents that release oligosaccharides from glycopeptides. In some embodiments, the oligosaccharide content of the composition is >2% (w/w). In some embodiments, the peptide content of the composition is >50% (w/w). In some embodiments, the peptide content of the composition is >40% (w/w). In some embodiments, the free amino acid content of the composition is <44% (w/w). In some embodiments, the free amino acid content of the composition is between 33% (w/w) and 43% (w/w).

In some embodiments, the composition comprises at least one glycoprotein- or glycopeptide-bound oligosaccharide of each of the following general formulae: Hex₁HexNAc₁, HexNAc₂, NeuAc₁HexNAc₁, NeuGc₁HexNAc₁, Hex₁HexNAc₁Fuc₁, Hex₁HexNAc₂, Hex₁HexNAc₂Sul₁, NeuAc₁Hex₁HexNAc₁, NeuGc₁Hex₁HexNAc₁, NeuAc₁HexNAc₂, NeuGc₁HexNAc₂, Hex₁HexNAc₂Fuc₁, Hex₁HexNAc₂Fuc₁Sul₁, NeuAc₁Hex₁HexNAc₁Fuc₁, Hex₁HexNAc₃Sul₁, Hex₂HexNAc₂Fuc₁, Hex₁HexNAc₃Fuc₁Sul₁, and Hex₂HexNAc₂Fuc₂Sul₁. In some embodiments, the composition comprises at least one glycopeptide-bound oligosaccharide of each of the following general formulae: Hex₁HexNAc₁, HexNAc₂, NeuAc₁HexNAc₁, NeuGc₁HexNAc₁, Hex₁HexNAc₁Fuc₁, Hex₁HexNAc₂, Hex₁HexNAc₂Sul₁, NeuAc₁Hex₁HexNAc₁, NeuGc₁Hex₁HexNAc₁, NeuAc₁HexNAc₂, NeuGc₁HexNAc₂, Hex₁HexNAc₂Fuc₁, Hex₁HexNAc₂Fuc₁Sul₁, NeuAc₁Hex₁HexNAc₁Fuc₁, Hex₁HexNAc₃Sul₁, Hex₂HexNAc₂Fuc₁, Hex₁HexNAc₃Fuc₁Sul₁, and Hex₂HexNAc₂Fuc₂Sul₁.

In some embodiments, the composition has a water solubility of 80-120 g/L at 25° C. In some embodiments, the composition has a water solubility of greater than 120 g/L at 25° C. In some embodiments, the composition does not substantially contain insoluble particles having a diameter greater than 7 μm. In some embodiments, the oligosaccharide content of the composition is >5% (w/w).

In some embodiments, the composition comprises glycoprotein- or glycopeptide-bound oligosaccharides, or glycopeptide-bound oligosaccharides, having at least 7 different structures selected from: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6 SGlcNAcβ1-6)GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3 GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3 [(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3 [Fucα1-2Galβ1-3(6S-)GlcNAcβ1-6]GalNAc, and Fucα1-2Galβ1-3 [Fucα1-2Galβ1-4(6S)GlcNAcβ1-6]GalNAc. In some embodiments, the composition comprises glycoprotein- or glycopeptide-bound oligosaccharides, or glycopeptide-bound oligosaccharides, having at least 14 different structures selected from the list of structures set forth above. In some embodiments, the composition comprises glycoprotein- or glycopeptide-bound oligosaccharides, or glycopeptide-bound oligosaccharides, having at least 21 different structures selected from the list of structures set forth above. In some embodiments, the composition comprises at least one glycoprotein- or glycopeptide-bound oligosaccharide, or at least one glycopeptide-bound oligosaccharide, having each structure shown above. In some embodiments, the composition comprises glycoprotein- or glycopeptide-bound oligosaccharides, or glycopeptide-bound oligosaccharides, having at least 28 different structures.

In some embodiments, the composition comprises at least one sialylated glycoprotein- or glycopeptide-bound oligosaccharide, or at least one sialylated glycopeptide-bound oligosaccharides. In some embodiments, the composition comprises at least three sialylated glycoprotein- or glycopeptide-bound oligosaccharides, or at least three sialylated glycopeptide-bound oligosaccharides. In some embodiments, the composition comprises at least six sialylated glycoprotein- or glycopeptide-bound oligosaccharides, or at least six sialylated glycopeptide-bound oligosaccharides. In some embodiments, the composition comprises ten sialylated glycoprotein- or glycopeptide-bound oligosaccharides, or at least ten sialylated glycopeptide-bound oligosaccharides. In some embodiments, the sialylated glycoprotein- or glycopeptide-bound oligosaccharides, or glycopeptide-bound oligosaccharides, are selected from the following: NeuAcα2-6GalNAc, NeuGcα2-6GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the sialylated glycoprotein- or glycopeptide-bound oligosaccharides, or glycopeptide-bound oligosaccharides, have the structures shown in FIGS. 18-19. In some embodiments, the composition comprises at least 1, 2, 3, 4, 5, 6, 7, 8, 9, or all 10 sialylated glycoprotein- or glycopeptide-bound oligosaccharides, or glycopeptide-bound oligosaccharides, having the structures shown in FIGS. 18-19.

In some embodiments, the oligosaccharide content of the composition is >10% (w/w). In some embodiments, the oligosaccharide content of the composition is >5% (w/w). In some embodiments, the free amino acid content of the composition is <10% (w/w). In some embodiments, the composition has less than 1% free glycans (w/w). In some embodiments, the composition has less than 0.1% free glycans (w/w). In some embodiments, the composition has less than 0.01% free glycans (w/w). In some embodiments, the composition has substantially no free glycans (w/w).

In some embodiments, the composition is capable of inhibiting glycan-mediated binding of one or more pathogenic micro-organisms to the epithelial cells of the gut (i.e., intestinal epithelial cells) when orally administered to a subject. In some embodiments, the one or more pathogenic microorganisms comprise Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, the composition is capable of reducing inflammation when orally administered to a subject. In some embodiments, reducing inflammation comprises a reduction in calprotectin in the blood stream or stool of the subject. In some embodiments, the composition, when orally administered to a subject, is capable of increasing short-chain fatty acid (SCFA) production in the gut of the subject. In some embodiments, the composition, when orally administered to a subject, is capable of lowering pH in the gut of the subject. In some embodiments, the decrease in pH is caused by an increase in SCFA production in the gut.

In some embodiments, the composition, when orally administered to a subject, is capable of increasing the growth or level of one or more commensal bacteria in the gut of the subject. In some embodiments, the obtained composition comprising a mixture of glycopeptides causes more growth of commensal bacteria when orally administered to a subject than an equivalent composition further treated to comprise a mixture of free glycans instead of a mixture of glycopeptides. In some embodiments, the one or more commensal bacteria comprise Coprococcus comes, Prevotella copri, Megamonas spp., or Bacteroides vulgatus.

In some embodiments, the gastrointestinal tract mucins are porcine gastrointestinal tract mucins. In some embodiments, the composition is for use as a medicament. In some embodiments, the composition is in the form of a powder (also sometimes referred to herein as a dried powder), a slurry, or a liquid.

Some aspects of the disclosure are directed to a nutritional or dietary composition or nutritional or dietary premix comprising a composition described herein. In some embodiments, the nutritional or dietary composition or nutritional or dietary premix is used to supplement an animal feed (e.g., a pet food, a dog food or treat, a cat food or treat, a livestock feed). Some aspects of the disclosure are directed to a pharmaceutical composition comprising at least one composition described herein and a pharmaceutically acceptable carrier, diluent or excipient. Some aspects of the disclosure are directed to a composition as described herein for use in prevention and/or treatment of an unbalance of the microbiota and/or disorders associated with dysbiosis such as asymptomatic dysbiotic microbiota, in particular depleted Akkermansia muciniphila gut microbiota. In some embodiments, the pharmaceutical composition is for use in animals (e.g., livestock animals or companion animals, dogs, cats).

Some aspects of the disclosure are directed to an animal feed comprising a composition described herein. In some embodiments, the animal feed comprises 0.5% to 2.0% w/w of the composition. In some embodiments, the animal feed is a dog food, a dog treat, a cat food, or a cat treat. In some embodiments, the animal feed is a livestock feed (e.g., pig feed, poultry feed).

Some aspects of the disclosure are directed to a method of manufacturing a composition comprising a mixture of glycopeptides, comprising the following steps a)-d): step a) providing gastrointestinal tract mucins or a partially purified fraction thereof having a pH of approximately 5.5, step b) optionally concentrating the mucins from step a) by evaporation, step c) partially removing substances in the mucins having a diameter of less than about 0.2 μm or less than about 0.45 μm by filtration or centrifugation, and step d) removing substances in the mucins having a diameter of greater than 7 μm by filtration or centrifugation.

As used herein, “a partially purified fraction” of gastrointestinal tract mucins comprises at least about 40%, at least about 50%, at least about 60%, at least about 70%, at least about 80%, at least about 90%, at least about 92.5%, at least about 95%, at least about 97.5%, at least about 98%, at least about 99%, or at least about 99.5% of the protein- and peptide-bound glycans present in un-purified gastrointestinal tract mucins.

In some embodiments, step a) further comprises purifying the mucins to remove large insoluble particles, fats, and lipids. In some embodiments, step a) further comprises desalinating the mucins.

In some embodiments, the method of manufacture further comprises a step e) of: further purifying the mucins by ultrafiltration, thereby removing particles having a weight of less than about 2 kDa. In some embodiments, the method of manufacture further comprises a step e) of: further purifying the mucins by removing substances in the mucins having a diameter of greater than about 0.22 μm by filtration or centrifugation. In some embodiments, the method of manufacture further comprises a step f) of: drying the resultant composition comprising the mixture of glycopeptides. In some embodiments, the resultant composition is dried via spray drying. Methods of spray drying are known in the art and are not limited.

In some embodiments, the resulting composition (i.e., obtained composition) comprising a mixture of glycopeptides has a water solubility of 80-120 g/L at 25° C. In some embodiments, the resulting composition (i.e., the obtained composition) comprising a mixture of glycopeptides has a water solubility of greater than or equal to about 120 g/L at 25° C. In some embodiments, the oligosaccharide content of the resulting composition comprising a mixture of glycopeptides is >5% (w/w).

In some embodiments, the resulting composition comprises glycoprotein- or glycopeptide-bound oligosaccharides, or glycopeptide-bound oligosaccharides, having 7 different structures selected from: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3 [Fucα1-2Galβ1-3(6S-)GlcNAcβ1-6]GalNAc, Fucα1-2Galβ1-3[Fucα1-2Galβ1-4(6S)GlcNAcβ1-6]GalNAc. In some embodiments, the resulting composition comprises glycoprotein- or glycopeptide-bound oligosaccharides, or glycopeptide-bound oligosaccharides, having at least 14 different structures selected from the list of structures shown above. In some embodiments, the resulting composition comprises glycoprotein- or glycopeptide-bound oligosaccharides, or glycopeptide-bound oligosaccharides, having at least 21 different structures selected from the list of structures shown above. In some embodiments, the resulting composition comprises at least one glycoprotein- or glycopeptide-bound oligosaccharide, or at least one glycopeptide-bound oligosaccharide, having each structure shown above.

In some embodiments, the resulting composition comprising a mixture of glycopeptides comprises glycopeptide-bound oligosaccharides having at least 28, 29, or 30 different structures. In some embodiments, the resulting composition comprising a mixture of glycopeptides comprises less than 1% free glycans (w/w). In some embodiments, the resulting composition comprising a mixture of glycopeptides comprises less than 0.1% free glycans (w/w). In some embodiments, the resulting composition comprising a mixture of glycopeptides comprises less than 0.01% free glycans (w/w). In some embodiments, the resulting composition comprising a mixture of glycopeptides comprises substantially no free glycans (w/w). The phrase “free glycans” refers to glycans that are not attached to a protein or polypeptide.

In some embodiments, the partially purified fraction of mucins of step a) comprises less than 1% free glycans. In some embodiments, the partially purified fraction of mucins of step a) comprises at least one glycoprotein- or glycopeptide-bound oligosaccharide, or at least one glycopeptide-bound oligosaccharide, of each of the following general formulae: Hex₁HexNAc₁, HexNAc₂, NeuAc₁HexNAc₁, NeuGc₁HexNAc₁, Hex₁HexNAc₁Fuc₁, Hex₁HexNAc₂, Hex₁HexNAc₂Sul₁, NeuAc₁Hex₁HexNAc₁, NeuGc₁Hex₁HexNAc₁, NeuAc₁HexNAc₂, NeuGc₁HexNAc₂, Hex₁HexNAc₂Fuc₁, Hex₁HexNAc₂Fuc₁Sul₁, NeuAc₁Hex₁HexNAc₁Fuc₁, Hex₁HexNAc₃Sul₁, Hex₂HexNAc₂Fuc₁, Hex₁HexNAc₃Fuc₁Sul₁, and Hex₂HexNAc₂Fuc₂Sul₁. As used herein, a “partially purified fraction” of mucins of step a) refer to a fraction of mucins comprising glycoproteins and glycopeptides, and not comprising more than about 5% free glycans. In some embodiments, the “partially purified fraction” of mucins of step a) does not comprise more than about 5%, more than about 4%, more than about 3%, more than about 2%, more than about 1%, more than about 0.5%, or more than about 0.1% free glycans. In some embodiments, the “partially purified fraction” of mucins of step a) comprises substantially no glycans. In some embodiments, the partially purified fraction of mucins of step a) has been partially depleted of glycans by enzymatic hydrolysis. In some embodiments, the mucins of step a) have been hydrolyzed. In some embodiments, the gastrointestinal tract mucins are porcine gastrointestinal tract mucins. In some embodiments, the partially purified fraction of mucins of step a) do not comprise substantially any glycoproteins.

In some embodiments, the resulting composition comprising a mixture of glycopeptides causes reduced growth or a reduced level of Escherichia coli in the gut of a subject (e.g., a dog, a cat, a human) when orally administered to the subject. In some embodiments, the resulting composition comprising a mixture of glycopeptides causes reduced growth of Escherichia coli when orally administered to a subject than a composition derived from the same process but not purified to remove insoluble particles greater than 7 μm. In some embodiments, the resulting composition comprising a mixture of glycopeptides causes increased growth of Akkermansia muciniphila gut microbiota when orally administered to a subject.

In some embodiments, the resulting or obtained composition comprising a mixture of glycopeptides causes more growth of commensal bacteria when orally administered to a subject than an equivalent composition further treated to comprise a mixture of free glycans instead of a mixture of glycopeptides. In some embodiments, the one or more commensal bacteria comprise Coprococcus comes, Prevotella copri, Megamonas spp., or Bacteroides vulgatus.

In some embodiments, the method of manufacture further comprises a step g) of adding the composition to a foodstuff. In some embodiments, the resultant foodstuff contains 0.5% to 2.0% w/w of the composition. In some embodiments, the foodstuff is an animal feed. In some embodiments, the animal feed is a dog food, a dog treat, a cat food, or a cat treat. In some embodiments, the animal feed is a livestock feed (e.g., pig feed, poultry feed).

Some aspects of the present invention are related to a method of treating, preventing, or reducing the severity of a pathogenic microorganism infection of the gut of a subject comprising orally administering to the subject a composition disclosed herein or a composition manufactured by a method disclosed herein. In some embodiments, the pathogenic microorganism is selected from Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, the pathogenic microorganism is Escherichia coli (e.g., a pathogenic strain of Escherichia coli).

Some aspects of the present invention are related to a method of increasing the growth of commensal bacteria in the gut of a subject comprising orally administering to the subject a composition disclosed herein or a composition manufactured by a method disclosed herein. In some embodiments, the commensal bacteria comprise Coprococcus comes, Prevotella copri, or Bacteroides vulgatus.

Some aspects of the present invention are related to a method of reducing the fat mass of a subject comprising orally administering to the subject a composition disclosed herein or a composition manufactured by a method disclosed herein.

Some aspects of the present invention are related to a method of treating, preventing, or reducing inflammation in a subject comprising orally administering to the subject a composition disclosed herein or a composition manufactured by a method disclosed herein. In some embodiments, reduces a level of calprotectin in the blood stream or stool of the subject.

Some aspects of the present invention are related to a method of increasing production of short chain fatty acid (SCFA) in the gut of a subject comprising orally administering to the subject a composition disclosed herein or a composition manufactured by a method disclosed herein. In some embodiments, the composition, when orally administered to a subject, is capable of lowering pH in the gut of the subject. In some embodiments, the decrease in pH is caused by an increase in SCFA production in the gut.

Some aspects of the present invention are related to a method of improving gut barrier integrity in the gut of a subject comprising orally administering to the subject a composition disclosed herein or a composition manufactured by a method disclosed herein.

Some aspects of the disclosure are directed to a composition comprising a mixture of glycopeptides obtainable from a method disclosed herein.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N J, 2005. Non-limiting information regarding therapeutic agents and human diseases is found in Goodman and Gilman's The Pharmacological Basis of Therapeutics, 11th Ed., McGraw Hill, 2005, Katzung, Bacteroides (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, MD) and National Center for Biotechnology Information, National Library of Medicine (Bethesda, MD), as of May 1, 2010, available on the World Wide Web at ncbi.nlm.nih.gov/omim/ and in Online Mendelian Inheritance in Animals (OMIA), a database of genes, inherited disorders and traits in animal species (other than human and mouse), at omia.angis.org.au/contact.shtml.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a liquid chromatography plot of a composition of the claimed invention (GNU100).

FIG. 2 shows a GNU100 profile obtained in HPAEC-PAD. The principal Sugars in the oligosaccharide component are shown.

FIG. 3 is a graph illustrating growth, as measured by OD on the y-axis, of Bifidobacterium bifidum at the indicated time points in minimal media without supplementation (no glucose), with glucose (glucose), or with a composition of the claimed invention (GNU100).

FIG. 4 is a graph illustrating growth, as measured by OD on the y-axis, of Bifidobacterium animalis subsp. lactis at the indicated time points in minimal media without supplementation (no glucose), with glucose (glucose), or with a composition of the claimed invention (GNU100).

FIG. 5 is a graph illustrating growth, as measured by OD on the y-axis, of Bifidobacterium breve at the indicated time points in minimal media without supplementation (no glucose), with glucose (glucose), or with a composition of the claimed invention (GNU100).

FIG. 6 is a graph illustrating growth, as measured by OD on the y-axis, of Lactobacillus acidophilus at the indicated time points in minimal media without supplementation (NG), with glucose (G), or with a composition of the claimed invention (GNU100).

FIG. 7 is a graph illustrating growth, as measured by OD on the y-axis, of Akkermansia muciniphila at the indicated time points in minimal media without supplementation (NG), with glucose (G), or with a composition of the claimed invention (GNU100).

FIG. 8 is a graph illustrating growth, as measured by OD on the y-axis, of Bacteroides thetaiotaomicron at the indicated time points in minimal media without supplementation (NG), with glucose (G), or with a composition of the claimed invention (GNU100).

FIG. 9 is a schematic of a process for obtaining a composition of the claimed invention.

FIG. 10 is a graph illustrating the number of dogs that consumed 0-20%, 21-40%, 41-60%, 61-80%, or 81-100% of dog food (standard diet+5% fat) supplemented with 1% of a composition of the claimed invention (GNU100).

FIG. 11 shows graphs illustrating total daily consumption and individual preferences of dogs for dog food (standard diet+5% fat) supplemented with 1% of a composition of the claimed invention (GNU100). Top panel shows that the group of dogs ate, in total, significantly more dog food (standard diet+5% fat) supplemented with 1% of a composition of the claimed invention (GNU100), than non-supplemented dog food. Bottom panel shows the preference of individual dogs for dog food (standard diet+5% fat) or dog food (standard diet+5% fat) supplemented with 1% of a composition of the claimed invention (GNU100).

FIG. 12 is a graph illustrating the number of cats that consumed 0-20%, 21-40%, 41-60%, 61-80%, or 81-100% of cat food supplemented with 1% of a composition of the claimed invention (GNU100).

FIG. 13 shows graphs illustrating total daily consumption and individual preferences of cats for cat food supplemented with 1% of a composition of the claimed invention (GNU100). Top panel shows that the group of cats ate, in total, significantly more cat food supplemented with 1% of a composition of the claimed invention (GNU100) than non-supplemented cat food. Bottom panel shows the preference of individual cats for cat food, or cat food supplemented with 1% of a composition of the claimed invention (GNU100).

FIG. 14 shows a comparison between traditional prebiotics having simple structures and galactose or fructose building blocks, and GNU100 having multiple building blocks, branched structures and a higher variety of structures (diversity) enabling wider functionality including for immune cell modulation and anti-microbial activity.

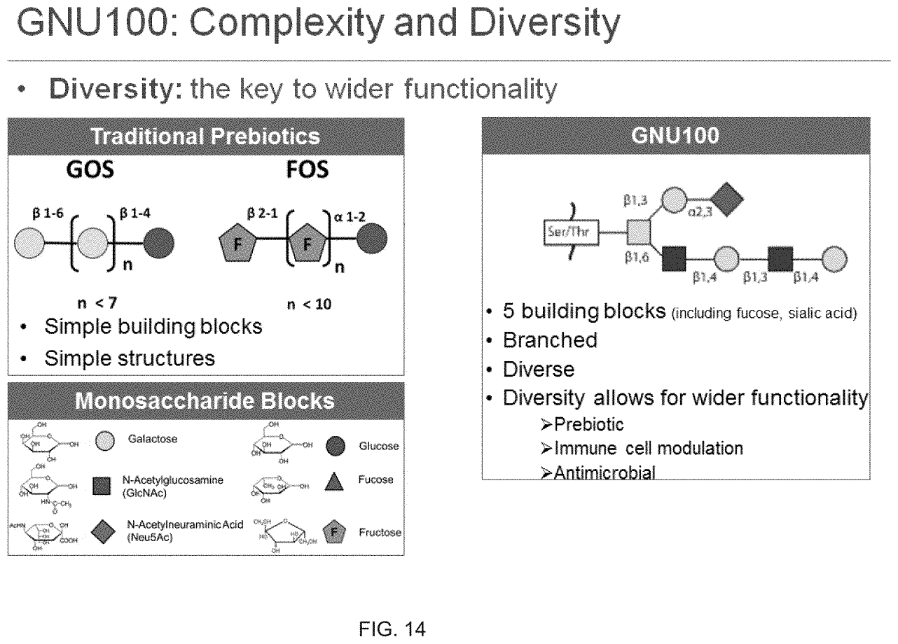

FIG. 15 shows the high structural diversity of GNU100 leads to greater functionality similar to natural milk oligosaccharides.

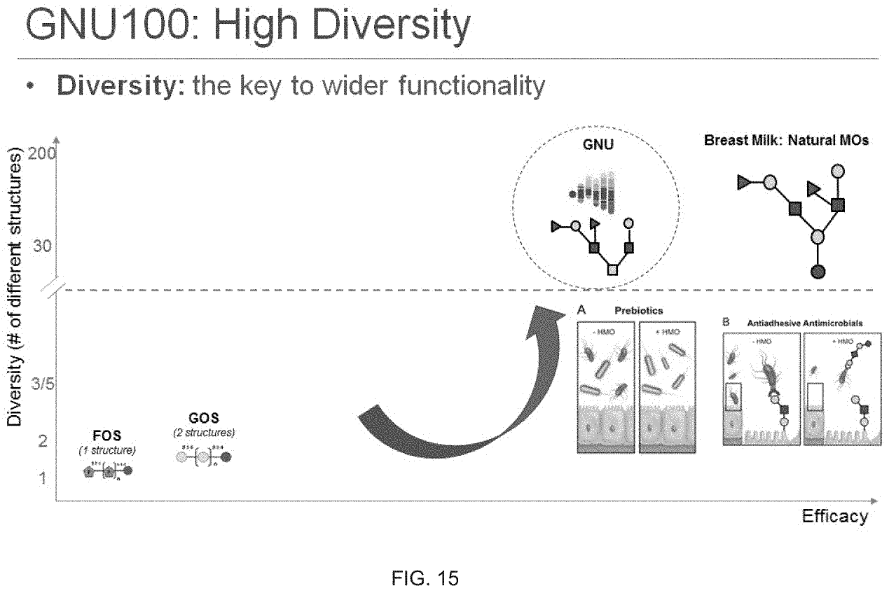

FIG. 16 shows that GNU100 contains sialyation: fucose and sialic acid residues that may be recognized by pathogens. GNU100 oligosaccharides are expected to bind to leptin receptors on the pathogens, thereby preventing binding to epithelial cells of the gut.

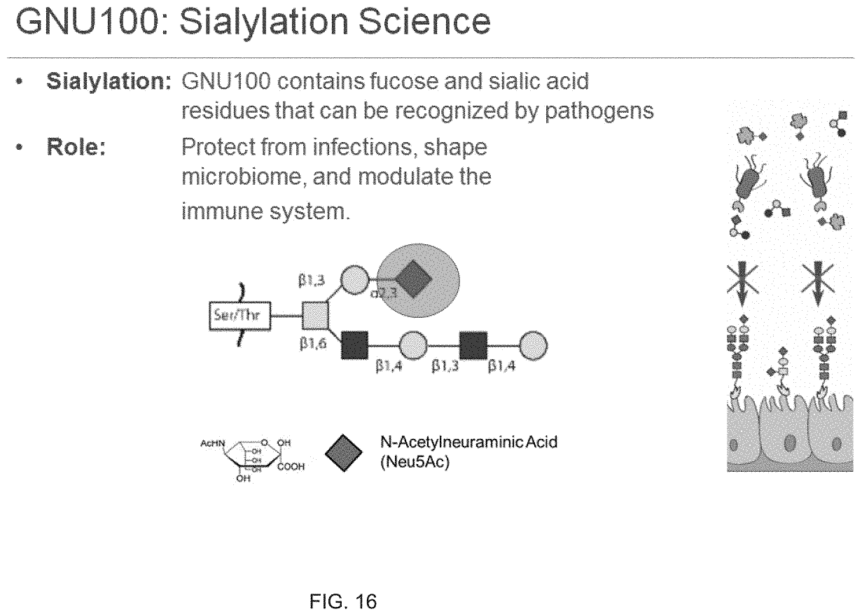

FIG. 17 shows GNU100 can comprise 10 sialylated glycans, similar to dog and cat milk. Conversely, neither FOS nor GOS comprise sialic acid residues and have less diversity and less protection against pathogens.

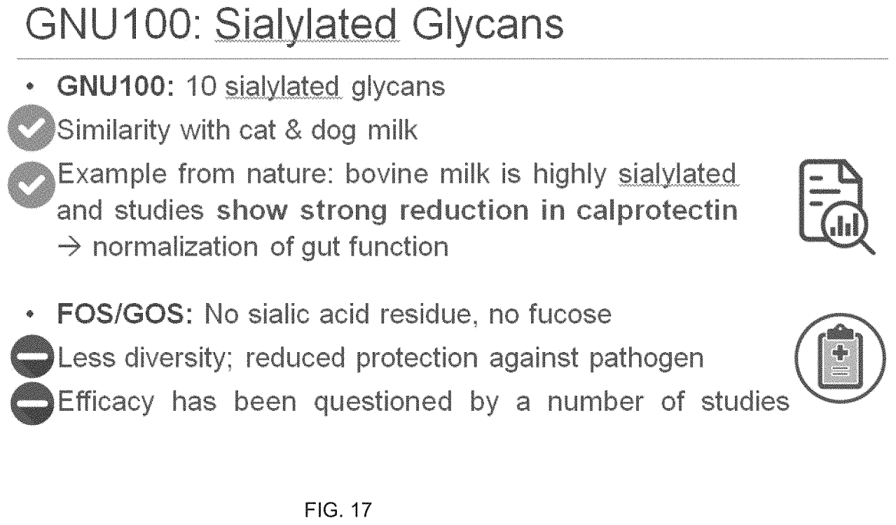

FIG. 18 shows sialylated glycan structures of glycoprotein- or glycopeptide-bound oligosaccharides of the present invention. The number at the top of each structure corresponds to the number provided in the first column of Table 1 herein.

FIG. 19 shows further sialylated glycan structures of glycoprotein- or glycopeptide-bound oligosaccharides of the present invention. The number at the top of each structure corresponds to the number provided in the first column of Table 1 herein. The key for the coloured shapes shown in the drawings are given in FIG. 18.

FIG. 20 shows an alternate schematic of a method to obtain a composition as taught herein.

FIG. 21 is a bar graph showing pH changes in a colonic simulation between 0-6, 6-24, and 24-48 hours after addition of either 0.5% or 1% GNU100 with cat faecal inoculum (first three groups of columns) or dog faecal inoculum (second three groups of columns).

FIG. 22 is a graph showing total gas production in a colonic simulation between 0-6, 6-24, and 24-48 hours after addition of either 0.5% or 1% GNU100 with cat faecal inoculum (first three columns) or dog faecal inoculum (second three columns).

FIG. 23 is a graph showing total acetate production in a colonic simulation between 0-6, 6-24, and 24-48 hours after addition of either 0.5% or 1% GNU100 with cat faecal inoculum (first three columns) or dog faecal inoculum (second three columns).

FIG. 24 is a graph showing total propinate production in a colonic simulation between 0-6, 6-24, and 24-48 hours after addition of either 0.5% or 1% GNU100 with cat faecal inoculum (first three columns) or dog faecal inoculum (second three columns).

FIG. 25 provides a graph showing total butyrate production in a colonic simulation between 0-6, 6-24, and 24-48 hours after addition of either 0.5% or 1% GNU100 with cat faecal inoculum (first three columns) or dog faecal inoculum (second three columns).

FIG. 26 provides a graph of Coprococcus comes growth in cat lumen (i.e., cat faecal inoculum without mucus beads) showing increased Caprococcuscomes at 24 hours post addition of either 0.5% or 1% GNU100.

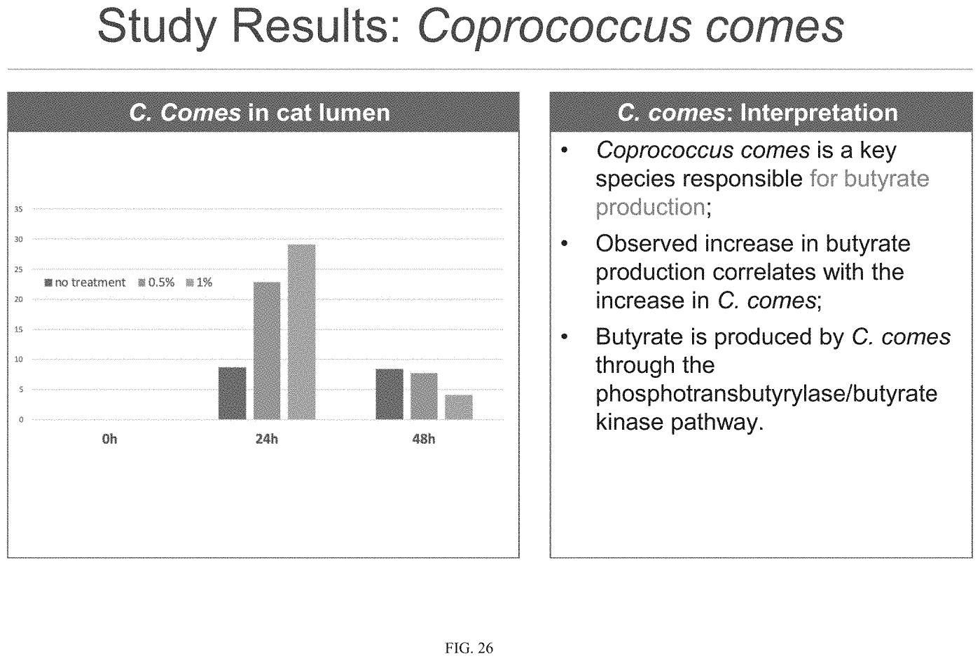

FIG. 27 provides a graph of Prevotella copri growth in dog lumen (i.e., dog faecal inoculum without mucus beads) showing increased Prevotella copri at 24 hours post addition of either 0.5% or 1% GNU100.

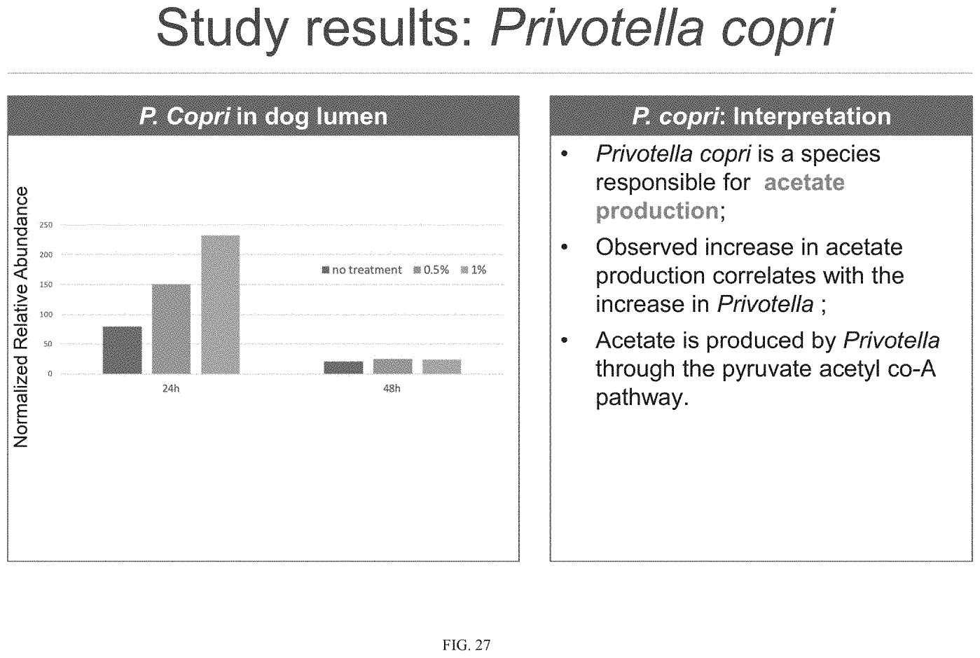

FIG. 28 provides a graph showing total lactic acid production in a colonic simulation (faecal inoculum) between 0-6, 6-24, and 24-48 hours after addition of either 0.5% or 1% GNU100 with cat faecal inoculum (first three columns) or dog faecal inoculum (second three columns).

FIG. 29 are graphs showing total ammonia production (top panel) or total SCFA production (bottom panel) in a colonic simulation between 0-6, 6-24, and 24-48 hours after addition of either 0.5% or 1% GNU100 with cat faecal inoculum (first three columns) or dog faecal inoculum (second three columns).

FIG. 30 is an illustration showing that GNU100 is a functional mimic of sugars found in milk, having prebiotic, immunomodulatory, anti-viral/anti-microbial, pathogen recognition, and immune function activity.

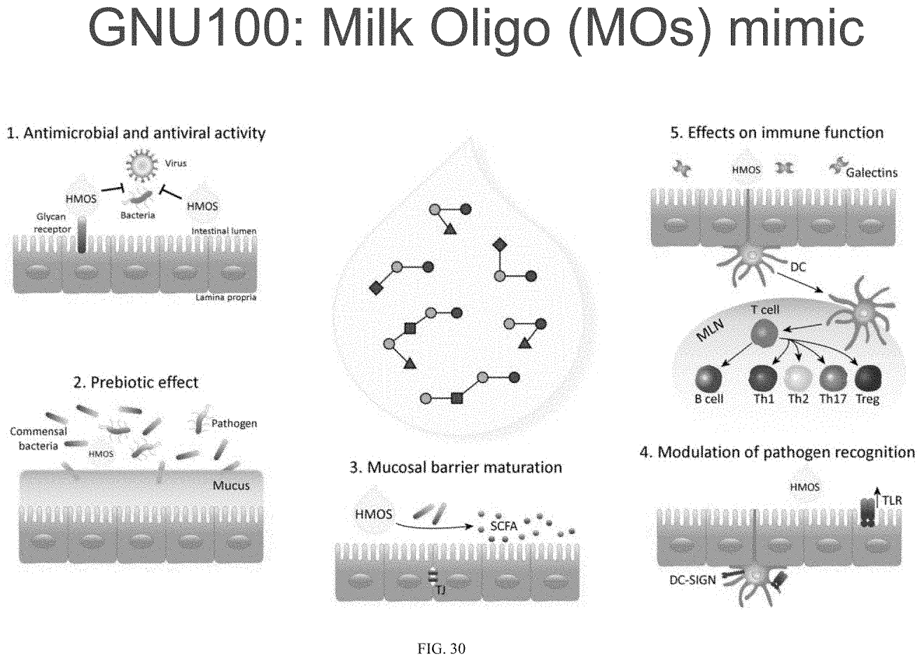

FIG. 31 provides a graph showing Bacteroides vulgatus growth in a colonic simulation with cat faecal inoculum with and without mucus beads with 0.5% or 1% GNU100 24 hours or 48 hours after addition of GNU100. 1% GNU100 increased Bacteroides vulgatus growth.

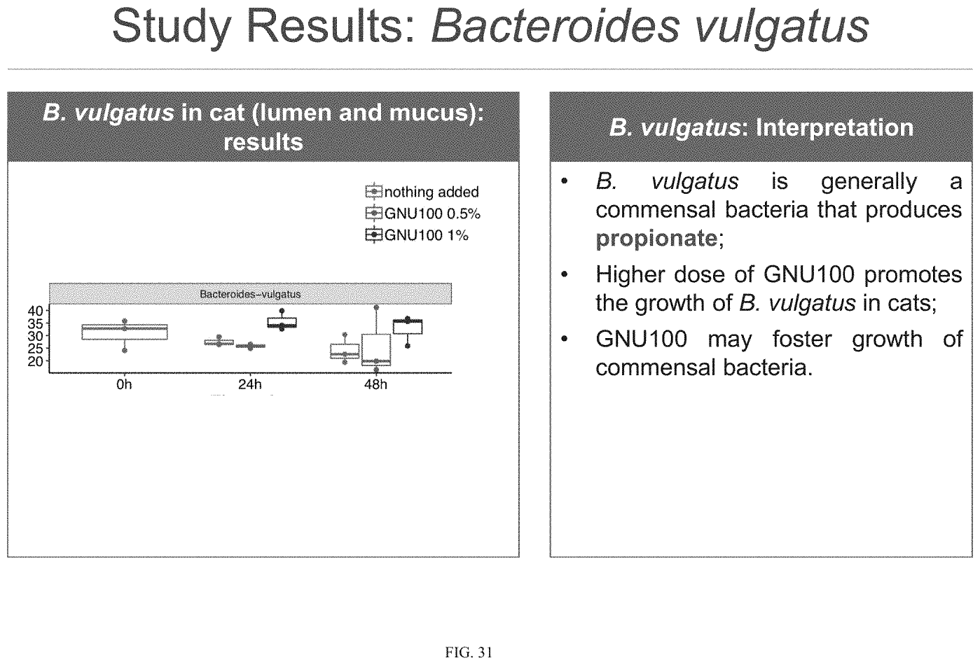

FIG. 32 provides a graph showing Escherichia coli growth in a colonic simulation with dog faecal inoculum with (mucus) and without (lumen) mucus beads. Escherichia coli growth was inhibited in a dose response curve.

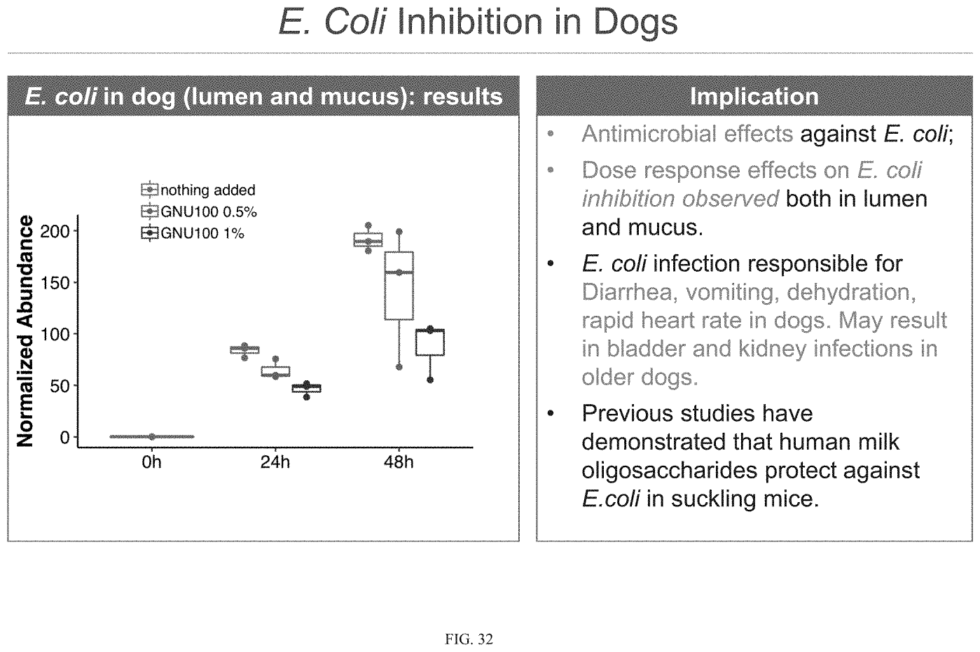

FIG. 33 provides a graph showing Escherichia coli growth in a colonic simulation with dog faecal inoculum with (mucus) and without (lumen) mucus beads. Escherichia coli growth was inhibited in the presence of mucus beads, suggesting GNU100 has an effect on the gut barrier in cats.

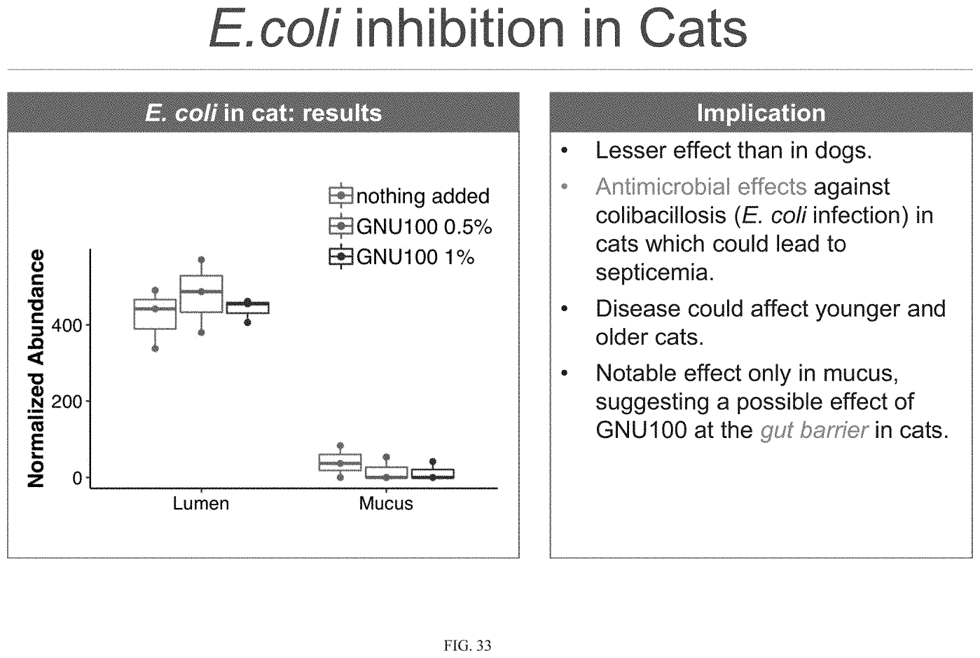

FIG. 34 is a schematic showing the simulator of intestinal tract environment with a faecal inoculum from dog or cat used in Example 8.

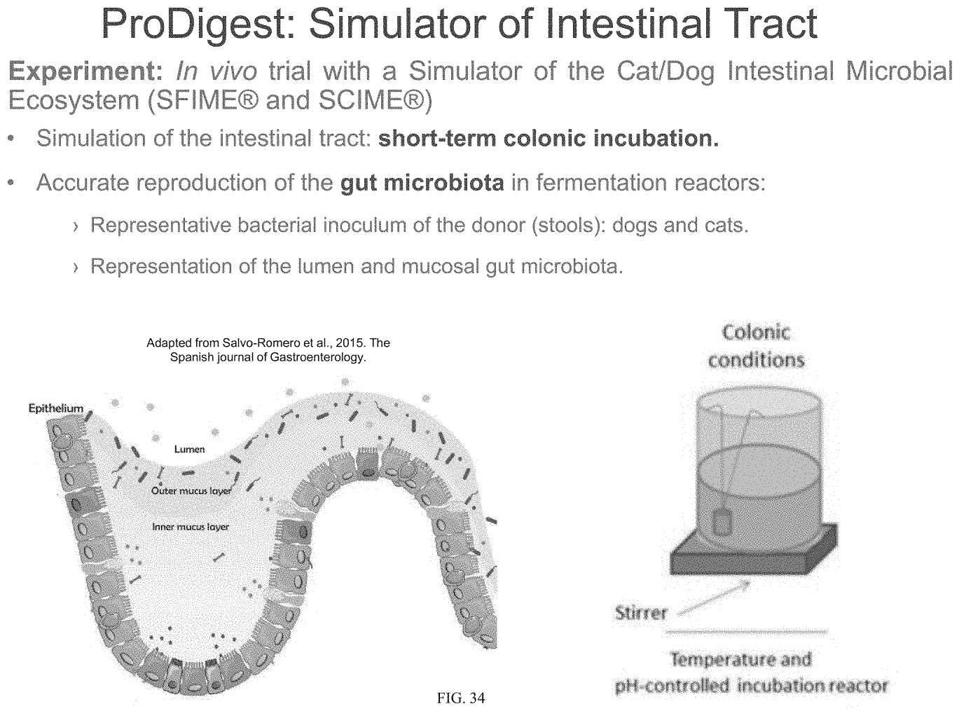

FIG. 35 is a schematic of the study design for the simulator of intestinal tract environment with a faecal inoculum from dog or cat used in Example 8.

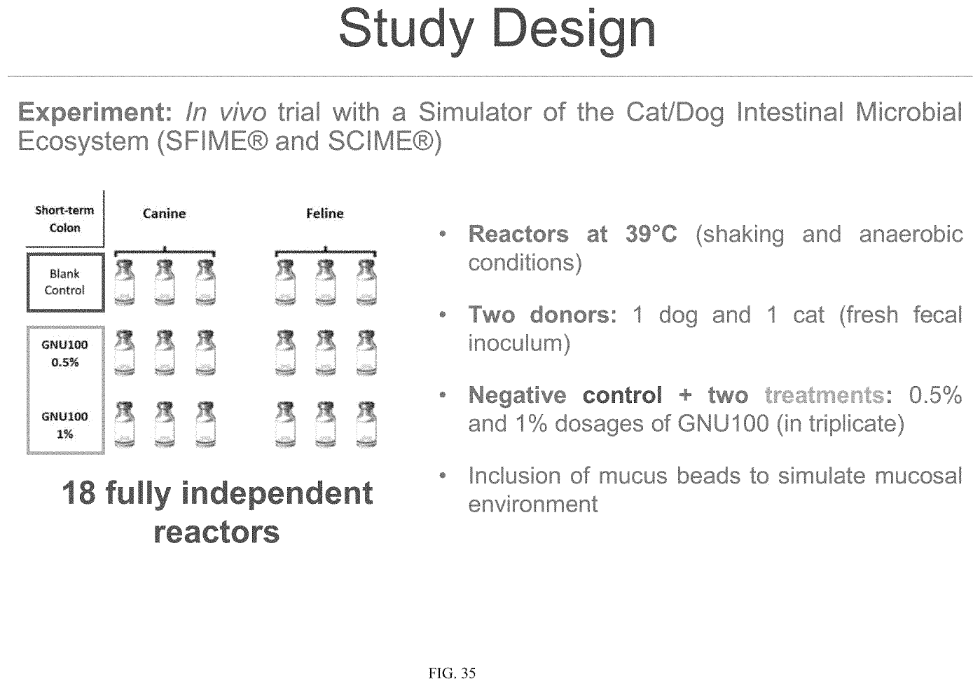

FIG. 36 shows normalized abundance of Escherichia coli detected 24 h and 48 h after the beginning of incubation in dog and cat lumen samples. Escherichia coli levels show a tendency to decrease after 24 h in dog samples treated with GNU100 0.5% (p=0.0536) and have a significant drop in samples treated with GNU100 1% (p=0.002337) compared to the control samples. The same trend was observed for the 48 h timepoint (pGNU100 0.5%=0.3289, pGNU100 1%=0.01251).

FIGS. 37A-37B show decreases in the relative abundance of Escherichia Spp in cat and dog samples with GNU100. (FIG. 37A) Escherichia spp abundance was greatly decreased in dog lumen samples treated with GNU100. This effect is dose-dependent and observable at 24 h and 48 h ABI. (FIG. 37B) A similar trend was observed in cat lumen samples despite GNU100 0.5% inducing a greater decrease than GNU100 1% at 24 h API.

FIG. 38 shows the normalized abundance of Prevotella copri in dog lumen samples. Prevotella copri increased with GNU100 addition to dog lumen samples at 24 h. An equally low abundance of Prevotella copri was observed for both treatment and control samples at 48 h ABI. Increase in Prevotella within the initial 24 hr corresponds to the release of SCFA.

FIG. 39 shows the relative abundance of the Clostridium in dog lumen samples. Clostridium was decreased in a dose-dependent manner at both 24 h and 48 h timepoints.

FIG. 40 shows the normalized abundance of Coprococcus comes in cat lumen samples. Coprococcus comes was increased in cat lumen samples treated with GNU100. This effect was dose-dependent and observable at 24 h. An equally low abundance was observed for both treatment and control samples at 48 h ABI.

FIGS. 41A-41B show the relative abundances of the Bacteroides genus and normalized abundance of Bacteroides vulgatus in cats and dog samples. (FIG. 41A) Bacteroides vulgatus levels show a tendency to increase at 24 h and 48 h ABI in cat samples treated with GNU100 1% (pGNU100 1% 24 h=0.005948, pGNU100 1% 48 h=0.003208) compared to the control samples. (FIG. 41B) Despite the overall low abundance, Bacteroides vulgatus is decreased in dog lumen samples at 48 h ABI (pGNU100 0.5% 48 h=0.009367, pGNU100 1% 48 h=0.003208) compared to the control samples. Error bars represent standard error of the mean.

FIGS. 42A-42B show the relative abundance of the Megamonas genus in cat and dog lumen samples. (FIG. 42A) Genus level analysis did not detect any member of the Megamonas genus in cat samples. (FIG. 42B) Megamonas abundance in dog lumen samples showed a dose-dependent increase in all samples treated with GNU100.

### DETAILED DESCRIPTION OF THE INVENTION

The expression “a source of gastrointestinal tract mucins” encompasses any natural source of mucin from which glycans and glycopetides can be extracted, suitable for mammalian nutrition or pharmaceutical use. Typical sources of gastrointestinal tract mucins are extracts from gastrointestinal tract, in particular from porcine source or from bovine source. Commercial sources for gastrointestinal tract mucins include Biofac A/S (Kastrup, Denmark), Zhongshi Duqing (Heze, China), Shenzhen Taier Biotechnology Co., LTD (Shenzhen, China), and Dongying Tiandong Pharmaceutical Co. (Shandong, China).

The expression “subject” refers to mammals. For examples, mammals contemplated by the present invention include human, primates, domesticated animals such as cattle, sheep, pigs, horses, rodents, cats, dogs and other pets. In preferred embodiments, the subject is a human. In other preferred embodiments, the subject is a dog or cat.

The expression “domestic animal” refers to cattle, sheep, pigs, horses, other farm mammals, rodents, cats, dogs and other pets.

The expression “nutritional supplement” means any comestible material having a nutritional value suitable for mammalian nutrition which can be used either alone as such or in combination with standard foodstuff.

The expression “feed additives” means products used in animal nutrition for purposes of improving the quality of feed and the quality of food from animal origin, or to improve the animals' performance and health, e.g. providing enhanced digestibility of the feed materials. In some embodiments, “feed additive” conforms with Article 5(3) of Regulation (EC) No 767/2009, section (f) “favorably affect animal production, performance or welfare, particularly by affecting the gastro-intestinal flora or digestibility of feed stuffs; or (g) have a coccidiostatic or histomonostatic effect.”

The expressions “animal food,” “animal feed,” and “pet food” means foodstuff suitable for animal nutrition. Substances such as nutrients and ingredients, in particular all the recommended vitamins and minerals suitable for nutritionally complete and balanced animal feed compositions, and recommenced amounts thereof, may be found for example, in the Official Publication of The Association of American Feed Control Officials, Inc. (AAFCO), Atlanta, G A, 2017 or in National Research Council, 2006, Nutritional Guidelines from the European Pet Food Industry Federation or Association of American Feed Control Officials, Official Publication, 2015. The expression “dog food” means foodstuff suitable for canine nutrition. The expression “cat food” means foodstuff suitable for feline nutrition. Dog food and cat food are known in the art to come in wet, semi-dry and dry formulations. Each of these wet, semi-dry and dry formulations are encompassed by “dog food” and “cat food” as disclosed herein unless it is otherwise apparent from the disclosure. In some embodiments, the dog food or cat food could be a treat. For instance, the “treat” could be a paste, biscuit, jerky treat, chewable flavored tablet, or the like. In some embodiments, the animal feed is a pig feed or a poultry (e.g., chicken, turkey) feed.

According to a particular embodiment, “dry” means that the water content is less than 5 weight-% (wt-%), based on the total weight of the composition, premix or formulation.

The term “glycoprotein” refers to proteins linked to oligosaccharides, e.g., proteins either N-linked or O-linked to oligosaccharides, and having a molecular weight of more than about 5 kDa. The term “glycopeptide” refers to peptides linked to oligosaccharides, e.g., peptides either N-linked or O-linked to oligosaccharides, and having a molecular weight of less than about 5 kDa. Methods of determining molecular weight of glycopeptides and glycoproteins are known in the art and are not limited. In some embodiments, the molecular weight of glycopeptides and glycoproteins are determined by size exclusion chromatography.

In some embodiments, peptides are defined as having a molecular weight of less than about 5 kDa. In some embodiments, the term peptides include glycopeptides. In some embodiments, proteins are defined as having a molecular weight of more than about 5 kDa. In some embodiments, the term proteins include glycoproteins.

### Compositions

Some aspects of the invention are directed to a composition comprising a mixture of glycopeptides obtained from gastrointestinal tract mucins, wherein the composition is obtained without subjecting the mucins or a partially purified fraction thereof to conditions or reagents that release oligosaccharides from glycoproteins or glycopeptides.

In some embodiments, the oligosaccharide content of the composition is greater than about 1.8% (w/w), greater than about 2.0% (w/w), greater than about 2.5% (w/w), greater than about 3% (w/w), greater than about 5% (w/w), greater than about 10% (w/w), greater than about 11% (w/w), greater than about 12% (w/w), greater than about 15% (w/w), greater than about 20% (w/w), or more. In some embodiments, the oligosaccharide content of the composition is greater than 5% (w/w). In some embodiments, the oligosaccharide content of the composition is greater than 10% (w/w). Methods of determining oligosaccharide content are known in the art and are not limited. In some embodiments, oligosaccharide content is determined by HPAEC-PAD with an acid pre-treatment to hydrolyze the glycans into monosaccharides.

In some embodiments, the peptide content of the composition is greater than about 65% (w/w), is greater than about 60% (w/w), is greater than about 55% (w/w), is greater than about 50% (w/w), is greater than about 45% (w/w), or is greater than about 40% (w/w). In some embodiments, the peptide content of the composition is greater than 50% (w/w). In some embodiments, the peptide content of the composition is greater than 40% (w/w). The peptide content as used herein refers to the content of all peptides, including glycopeptides. Methods of determining peptide content are known in the art and are not limited. In some embodiments, peptide content is determined by size exclusion chromatography.

In some embodiments, the protein content of the composition is less than about 0.05% (w/w), less than about 0.1% (w/w), less than about 1% (w/w), less than about 5% (w/w), less than about 6% (w/w), less than about 7% (w/w), less than about 8% (w/w), less than about 9% (w/w), less than about 10% (w/w), less than about 15% (w/w), or less than about 20% (w/w). In some embodiments, the protein content of the composition is less than 10% (w/w). In some embodiments, the composition is substantially free of protein. Methods of determining protein content are known in the art.

In some embodiments, the free amino acid content of the composition is less than about 44% (w/w), less than about 40% (w/w), is less than about 38% (w/w), is less than about 36% (w/w), is less than about 34% (w/w), is less than about 32% (w/w), is less than about 31% (w/w), is less than about 30% (w/w), less than about 29.5% (w/w), less than about 29% (w/w), less than about 28.5% (w/w), less than about 28% (w/w), less than about 27% (w/w), less than about 26% (w/w), less than about 25% (w/w), less than about 24% (w/w), less than about 20% (w/w), less than about 15% (w/w), less than about 10% (w/w), less than about 7.5% (w/w), less than about 5% (w/w), less than about 2.5% (w/w), less than about 1% (w/w), or less than about 0.5% (w/w). In some embodiments, the free amino acid content of the composition is less than 30% (w/w) or less than 10% (w/w). In some embodiments, the free amino acid content of the composition is less than 44% (w/w). In some embodiments, the free amino acid content of the composition is between 33% (w/w) and 43% (w/w). Methods of determining free amino acid content are known in the art. In some embodiments, free amino acid content is determined by Hydrophilic Interaction Liquid Chromatography coupled to High Resolution Mass Spectrometry (HILIC-HRMS). In some embodiments, free amino acid content is determined by HPLC, LC-MS/MS, HPAEC-PAD, and/or with an amino acid analyser.

In some embodiments, the composition comprises glycoprotein- or glycopeptide-bound oligosaccharides having each of the following general formulae: Hex₁HexNAc₁, HexNAc₂, NeuAc₁HexNAc₁, NeuGc₁HexNAc₁, Hex₁HexNAc₁Fuc₁, Hex₁HexNAc₂, Hex₁HexNAc₂Sul₁, NeuAc₁Hex₁HexNAc₁, NeuGc₁Hex₁HexNAc₁, NeuAc₁HexNAc₂, NeuGc₁HexNAc₂, Hex₁HexNAc₂Fuc₁, Hex₁HexNAc₂Fuc₁Sul₁, NeuAc₁Hex₁HexNAc₁Fuc₁, Hex₁HexNAc₃Sul₁, Hex₂HexNAc₂Fuc₁, Hex₁HexNAc₃Fuc₁Sul₁, and Hex₂HexNAc₂Fuc₂Sul₁. In some embodiments, the composition comprises glycopeptide-bound oligosaccharides having each of the following general formulae: Hex₁HexNAc₁, HexNAc₂, NeuAc₁HexNAc₁, NeuGc₁HexNAc₁, Hex₁HexNAc₁Fuc₁, Hex₁HexNAc₂, Hex₁HexNAc₂Sul₁, NeuAc₁Hex₁HexNAc₁, NeuGc₁Hex₁HexNAc₁, NeuAc₁HexNAc₂, NeuGc₁HexNAc₂, Hex₁HexNAc₂Fuc₁, Hex₁HexNAc₂Fuc₁Sul₁, NeuAc₁Hex₁HexNAc₁Fuc₁, Hex₁HexNAc₃Sul₁, Hex₂HexNAc₂Fuc₁, Hex₁HexNAc₃Fuc₁Sul₁, and Hex₂HexNAc₂Fuc₂Sul₁. In some embodiments, the composition further comprises at least one, at least two, at least three, at least four, at least five, at least six, at least seven, at least eight, at least nine, at least ten, at least fifteen, or at least twenty glycoprotein- or glycopeptide-bound oligosaccharides having a general formula that differs from any of the general formulae set forth above. In some embodiments, the composition further comprises at least one, at least two, at least three, at least four, at least five, at least six, at least seven, at least eight, at least nine, at least ten, at least fifteen, or at least twenty glycopeptide-bound oligosaccharides having a general formula that differs from any of the general formulae set forth above. Methods of determining the general formula of glycopeptide or glycoprotein bound oligosaccharides are known in the art. In some embodiments, the general formula of glycopeptide or glycoprotein bound oligosaccharides is determined by liquid chromatography-electrospray ionization tandem mass spectrometry (LC-ESI/MS) after reductive glycan release. In some embodiments, the composition comprises substantially no glycoproteins.

In some embodiments, the composition has a water solubility of 80-120 g/L at 25° C. In some embodiments, the composition has a water solubility of about 80 g/L, about 85 g/L, about 90 g/L, about 95 g/L, about 100 g/L, about 105 g/L, about 110 g/L, about 115 g/L, or about 120 g/L at 25° C. In some embodiments, the composition has a water solubility of greater than about 120 g/L at 25° C.

In some embodiments, the composition does not substantially contain insoluble particles having a diameter greater than 7 μm. As utilized herein, the term “substantially” refers to the complete or nearly complete extent or degree of a characteristic or property, as would be appreciated by one of skill in the art. Thus, a composition that “does not substantially contain insoluble particles having a diameter greater than 7 μm” refers to a composition having a lack of, or near lack of, insoluble particles with a diameter greater than 7 μm, as would be appreciated by one of skill in the art. For instance, if a composition is filtered to remove insoluble particles having a diameter greater than 7 μm, such composition may still contain a trace amount of insoluble particles having a diameter greater than 7 μm, but would be considered substantially free of insoluble particles having a diameter greater than 7 μm. In some embodiments, the composition does not substantially contain insoluble particles having a diameter greater than about 7 μm, greater than about 6 μm, greater than about 5 μm, or greater than about 4 μm. In some embodiments, the composition does not contain insoluble particles having a diameter greater than about 7 μm, greater than about 6 μm, greater than about 5 μm, or greater than about 4 μm. Methods of determining particle size are known in the art. In some embodiments, a filter with a desired cut-off size (for instance, 7 μm) can be used to remove insoluble particles larger than the cut-off size, or determine whether a composition contains insoluble particles greater than a desired cut-off size.

In some embodiments, the composition does not substantially contain insoluble particles having a diameter of greater than about 0.3 inn, greater than about 0.22 inn, or greater than about 0.1 inn. In some embodiments, the composition is filtered or centrifuged to remove insoluble particles having a diameter greater than 0.22 inn.

In some embodiments, the composition comprises glycoprotein- or glycopeptide-bound oligosaccharides having at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or all of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1- 6)GalNAc, Fucα1-2Galβ1-3(NeuAcβ2-6)GalNAc, GlcNAcβ1-3 [Galβ1-4(6 S)GlcNAcβ1-6]GalNAc, Galβ1-4GlcNAcβ1-3 [(6 S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, Fucα1-2Galβ1-3[Fucα1-2Galβ1-4(6S)GlcNAcβ1-6]GalNAc. In some embodiments, the composition comprises or glycopeptide-bound oligosaccharides having at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or all of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3 [(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3 [Fucα1-2Galβ1-3(6S-)GlcNAcβ1-6]GalNAc, Fucα1-2Galβ1-3 [Fucα1-2Galβ1-4(6S)GlcNAcβ1-6]GalNAc. In some embodiments, the composition comprises substantially no glycoproteins.

Methods of determining the structure of oligosaccharides bound to glycoproteins and glycopeptides are known in the art and are not limited. In some embodiments, the structure of oligosaccharides bound to glycoproteins and glycopeptides is determined by tandem mass spectrometry (MS/MS).

In some embodiments, the composition comprises glycoprotein- or glycopeptide-bound oligosaccharides having at least 14 different structures selected from the list of structures shown above. In some embodiments, the composition comprises glycopeptide-bound oligosaccharides having at least 14 different structures selected from the list of structures shown above.

In some embodiments, the composition comprises glycoprotein- or glycopeptide-bound oligosaccharides having at least 21 different structures selected from the list of structures shown above. In some embodiments, the composition comprises glycopeptide-bound oligosaccharides having at least 21 different structures selected from the list of structures shown above.

In some embodiments, the composition comprises at least one glycoprotein- or glycopeptide-bound oligosaccharide having each structure shown above. In some embodiments, the composition comprises at least one glycopeptide-bound oligosaccharide having each structure shown above.

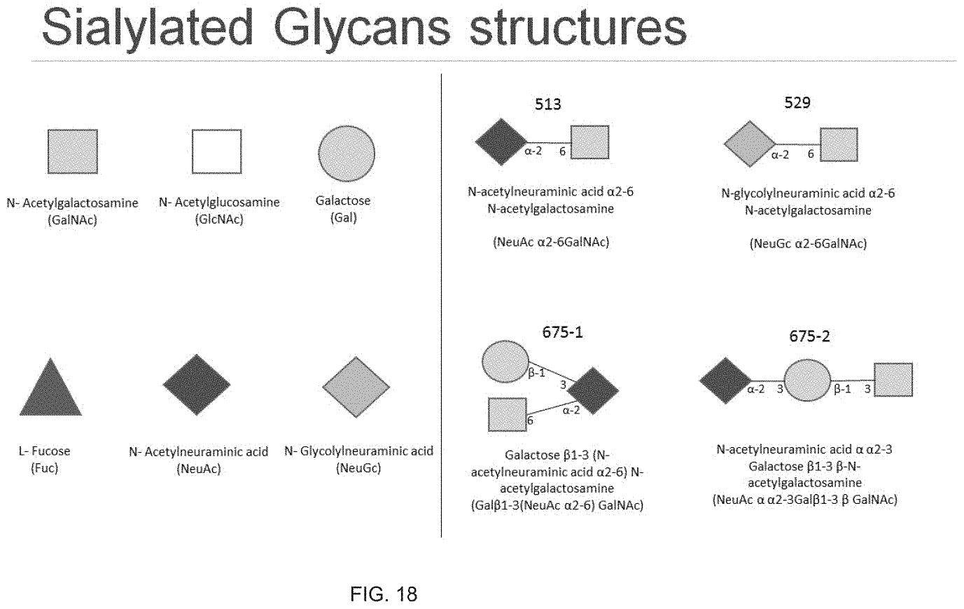

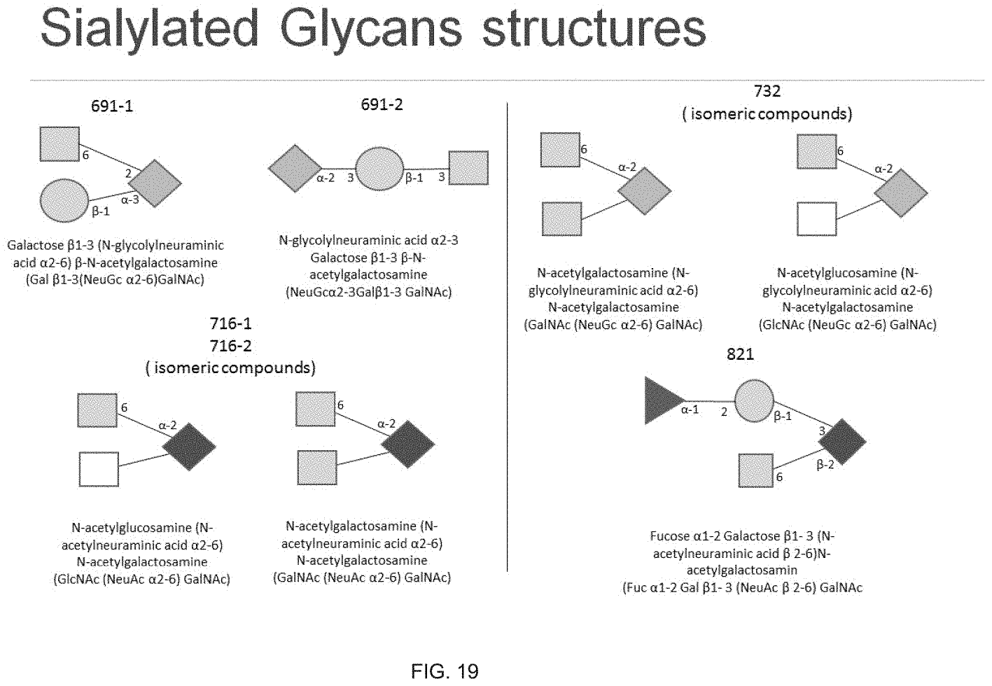

In some embodiments, the composition comprises at least one sialylated glycoprotein- or glycopeptide-bound oligosaccharide. In some embodiments, the composition comprises at least three sialylated glycoprotein- or glycopeptide-bound oligosaccharides. In some embodiments, the composition comprises at least six sialylated glycoprotein- or glycopeptide-bound oligosaccharides. In some embodiments, the composition comprises ten sialylated glycoprotein- or glycopeptide-bound oligosaccharides. In some embodiments, the sialylated glycoprotein- or glycopeptide-bound oligosaccharides are selected from the following: NeuAcα2-6GalNAc, NeuGcα2-6GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the sialylated glycoprotein- or glycopeptide-bound oligosaccharides have the structures shown in FIGS. 18-19. In some embodiments, the composition comprises at least 1, 2, 3, 4, 5, 6, 7, 8, 9, or all 10 sialylated glycoprotein- or glycopeptide-bound oligosaccharides having the structures shown in FIGS. 18-19.

In some embodiments, the composition comprises glycoprotein- or glycopeptide-bound oligosaccharides having at least 20, 21, 22, 23, 24, 25, 26, 27, 28, 29, 30, 31, 32, 33, 34, 35, 36, 37, 38, 39, or 40 different structures.

In some embodiments, the composition comprises at least one sialylated glycopeptide-bound oligosaccharide. In some embodiments, the composition comprises at least three sialylated glycopeptide-bound oligosaccharides. In some embodiments, the composition comprises at least six sialylated glycopeptide-bound oligosaccharides. In some embodiments, the composition comprises ten sialylated or glycopeptide-bound oligosaccharides. In some embodiments, the sialylated glycopeptide-bound oligosaccharides are selected from the following: NeuAcα2-6GalNAc, NeuGcα2-6GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, and Fucα1-2Galβ1-3(NeuAcβ2-6)GalNAc. In some embodiments, the sialylated glycopeptide-bound oligosaccharides have the structures shown in FIGS. 18-19. In some embodiments, the composition comprises at least 1, 2, 3, 4, 5, 6, 7, 8, 9, or all 10 sialylated glycopeptide-bound oligosaccharides having the structures shown in FIGS. 18-19.

In some embodiments, the composition comprises glycopeptide-bound oligosaccharides having at least 20, 21, 22, 23, 24, 25, 26, 27, 28, 29, 30, 31, 32, 33, 34, 35, 36, 37, 38, 39, or 40 different structures.

In some embodiments, the composition comprises less than about 5%, less than about 2.5%, less than about 1%, less than about 0.5%, less than about 0.1%, or less than about 0.01% free glycans (w/w). In some embodiments, the composition comprises substantially no free glycans. Methods of measuring free glycans are known in the art and are not limited. In some embodiments, free glycans are measured by LC-MS/MS (Liquid Chromatography with tandem mass spectrometry).

In some embodiments, the composition is capable of inhibiting glycan-mediated binding of one or more pathogenic micro-organisms to mucosal cells when orally administered to a subject. Many pathogens like bacteria, viruses and protozoan parasites, express lectins to attach to the glycans of the epithelial cell surface of the host and colonize or invade the host and cause disease. Sialylated glycans in GNU100 have structures similar to surface glycans of intestinal epithelial cells (i.e., epithelial cells of the gut). Thus, sialylated glycans in GNU100 can serve as bacterial lectin ligand analogs blocking bacterial attachment and act as antiadhesive antimicrobials. GNU100 structures can therefore serve as soluble decoy moieties to prevent pathogen binding and decrease the risk of infections as unbound pathogens are carried downstream and excreted with the feces. Alternatively, GNU100 structures can bind receptors such as lectins on host cells which could block pathogen binding to host cells via a competition mechanism, reducing risk of infections. The aforementioned mechanisms are not only relevant to gastrointestinal tract environment but also to other body locations that contain mucus such as, but not limited to, the respiratory or urinary tract.

In some embodiments, the one or more pathogenic microorganisms comprise Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition inhibits glycan-mediated binding of one or more pathogenic micro-organisms to mucosal cells by about 5%, 10%, 20%, 30%, 40%, 50%, 60%, 70%, 80%, 85%, 90%, 95%, 99%, 99.9% or more. In some embodiments, administration of the composition inhibits glycan-mediated binding of one or more pathogenic micro-organisms to mucosal cells by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition is capable of reducing the growth of one or more pathogenic microorganisms in the gut when administered to a subject. In some embodiments, the one or more pathogenic microorganisms comprise Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition inhibits the growth of the pathogenic microorganism by about 5%, 10%, 20%, 30%, 40%, 50%, 60%, 70%, 80%, 85%, 90%, 95%, 99%, 99.9% or more. In some embodiments, administration of the composition inhibits the growth of the pathogenic microorganism by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition is capable of reducing the level of one or more pathogenic microorganisms in the gut when administered to a subject. In some embodiments, the one or more pathogenic microorganisms comprise Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition reduces the level of the pathogenic microorganism in the gut by about 5%, 10%, 20%, 30%, 40%, 50%, 60%, 70%, 80%, 85%, 90%, 95%, 99%, 99.9% or more. In some embodiments, administration of the composition reduces the level of the pathogenic microorganism in the gut by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, the composition is capable of reducing the level of Escherichia coli (e.g., pathogenic Escherichia coli) in the gut when administered to a subject. In some embodiments, administration of the composition reduces the level of Escherichia coli in the gut by about 10%-80%, 20%-70%, 30%-60%, or any range therebetween.

In some embodiments, the composition is capable of reducing inflammation when orally administered to a subject. In some embodiments, administration of the composition reduces inflammation (e.g., inflammation in the gut) by about 5%, 10%, 20%, 30%, 40%, 50%, 60%, 70%, 80%, 85%, 90%, 95%, 99%, 99.9% or more. In some embodiments, administration of the composition reduces inflammation (e.g., inflammation in the gut) by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, reducing inflammation comprises a reduction in calprotectin in the blood stream or stool of the subject. In some embodiments, calprotectin is increased in the stool or decreased in the blood by about 5%, 10%, 20%, 30%, 40%, 50%, 60%, 70%, 80%, 85%, 90%, 95%, 99%, 99.9% or more. In some embodiments, calprotectin is increased in the stool or decreased in the blood by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition is capable of increasing lactate production in the gut when orally administered to a subject. In some embodiments, lactate production is increased by about 5%, 10%, 20%, 30%, 40%, 50%, 60%, 70%, 80%, 85%, 90%, 95%, 99%, 99.9% or more. In some embodiments, lactate production is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition, when orally administered to a subject, is capable of increasing short-chain fatty acid (SCFA) production in the gut of the subject. SCFAs play an important role in host health and SCFA production is considered a benefit to the host. SCFAs serve as a source of energy for intestinal epithelial cells, and help maintain intestinal integrity by promoting mucus production and gut barrier function. SCFAs also have anti-tumor effects on colonic carcinoma. SCFAs have further been shown to have immunomodulation effects including T cell regulation and intestinal anti-inflammatory properties. Moreover, SCFAs are involved in the modulation of homeostasis and metabolism including reduction of cholesterol and fatty acid synthesis in the liver. SCFAs have also been shown to have antibacterial properties via stimulating antimicrobial peptides and reducing luminal pH. In some embodiments, SCFAs comprise at least one of butyrate and propionate.

In some embodiments, SCFA production (e.g., butyrate and/or propionate) is increased by about 5%, 10%, 20%, 30%, 40%, 50%, 60%, 70%, 80%, 85%, 90%, 95%, 99%, 99.9% or more. In some embodiments, SCFA production (e.g., butyrate and/or propionate) is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition, when orally administered to a subject, is capable of lowering pH in the gut of the subject. Lowering the pH is advantageous as growth and viability of beneficial bacteria can be enhanced. In some embodiments, the decrease in pH is caused by an increase in SCFA production in the gut.

The source of gastrointestinal tract mucins is not limited. Gastrointestinal tract mucins can be obtained from bovine, porcine, ovine, dromedary, and avian sources. In some embodiments, the gastrointestinal tract mucins are porcine gastrointestinal tract mucins. In some embodiments, hydrolyzed porcine gastrointestinal tract mucins obtained as an industrial by-product from heparin production are used as a source of porcine gastrointestinal tract mucins. In some embodiments, the hydrolyzed porcine gastrointestinal tract mucins obtained as an industrial by-product from heparin production have been subjected to a proteolytic enzyme treatment to release heparin glycans. In some embodiments, the proteolytic enzyme is trypsin, chymotrypsin, papain, or subtilisin-type enzymes such as ALCALASE® or MAXATASE®. In some embodiments, the hydrolyzed porcine gastrointestinal tract mucins obtained as an industrial by-product from heparin production have been subjected to autolysis, addition of pancreas extract, saliva, or chemical hydrolysis to release heparin. In some embodiments, the hydrolyzed porcine gastrointestinal tract mucins obtained as an industrial by-product from heparin production have not been subjected to autolysis, addition of pancreas extract, saliva, or chemical hydrolysis to release heparin. In some embodiments, the hydrolyzed porcine gastrointestinal tract mucins obtained as an industrial by-product from heparin production have been treated with a subtilisin-type enzyme or quaternary ammonium resins to remove heparin.

In some embodiments, the composition is for use as a medicament. In some embodiments, the composition is for use in a nutritional or dietary composition or nutritional or dietary premix. In some embodiments, the nutritional or dietary composition or nutritional or dietary premix is for use in supplementing an animal feed. In some embodiments, the nutritional or dietary composition or nutritional or dietary premix is for use as a pet food supplement (e.g., to supplement a dog food, dog treat, cat food, or cat food treat). In some embodiments, the nutritional or dietary composition or nutritional or dietary premix is for use as a livestock (e.g. pig or poultry) feed supplement. The nutritional or dietary composition or nutritional or dietary premix may be in the form of a slurry, liquid, syrup, or powder. In some embodiments, the nutritional or dietary composition or nutritional or dietary premix does not contain additional flavoring agents to enhance palatability for the animal. As shown herein, dogs and cats find the compositions disclosed herein highly palatable (e.g., more palatable than standard dog or cat food).

In some embodiments, the composition is for use in a pharmaceutical composition further comprising a pharmaceutically acceptable carrier, diluent or excipient.

In some embodiments, the composition is for use in prevention and/or treatment of an unbalance of the microbiota and/or disorders associated with dysbiosis such as asymptomatic dysbiotic microbiota, in particular depleted Akkermansia muciniphila gut microbiota. The term “dysbiosis” is defined as a state in which the microbiota produces harmful effects via (a) qualitative and quantitative changes in the content or amount of the microbiota itself (e.g., depleted Akkermansia muciniphila), (b) changes in their metabolic activities; and/or (c) changes in their local distribution. Abnormalities in microbiota composition and activity (called dysbiosis) have been implicated in the emergence of the metabolic syndrome, which include diseases such as obesity, type 2 diabetes and cardiovascular diseases. Akkermansia muciniphila is one of the most abundant single species in the healthy human intestinal microbiota (0.5-5% of the total bacteria). Low levels of Akkermansia muciniphila in the dietary tract have been associated with insulin resistance and metabolic disease. Thus, in some embodiments, a human with dysbiosis has a percentage of Akkermansia muciniphila in the gut compared to total gut bacteria of less than about 3%, 2%, 1.5%, 1%, or 0.5%. In some embodiments, a human with dysbiosis exhibits insulin resistance or obesity. In some embodiments, the composition is for use in prevention and/or treatment of obesity. In some embodiments, the composition is for use in weight control.

In some embodiments, the composition is for use in prevention and/or treatment of Escherichia coli infection in a subject. In some embodiments, the subject is a cat with colibacillosis. In some embodiments, the subject has one or more of diarrhea, vomiting, dehydration, or rapid heartbeat. In some embodiments, the subject is a dog (e.g., elderly dog). In some embodiments, the composition is for use in prevention and/or treatment of a kidney or bladder infection.

In some embodiments, the composition is for use in an animal feed.

In some embodiments, the composition can be used for the preparation of nutritional/dietary supplement or complete food, in particular for oral delivery.

In some embodiments, the composition is in the form of nutritional supplement or complete food. In some embodiments, the composition is useful as an infant formula supplement. In some embodiments, the composition is useful as a human nutritional supplement. In some embodiments, the composition is useful as a domestic animal nutritional supplement. In some embodiments, the composition is useful as a dog or cat nutritional supplement. In some embodiments, the composition is useful as a livestock (e.g., pig, poultry) nutritional supplement.

The complete food or dietary/nutritional supplement according to the invention can be artificially enriched in vitamins, soluble or insoluble mineral salts or mixtures thereof or enzymes.

The compositions of the invention can be formulated as solid dosage forms containing a nutritional/dietary supplement with or without suitable excipients or diluents and prepared either by compression or molding methods well known in the art, encompassing compressed tablets and molded tablets or tablet triturates. In addition to the active or therapeutic/nutritional/cosmetic ingredient or ingredients, tablets contain a number or inert materials or additives, including those materials that help to impart satisfactory compression characteristics to the formulation, including diluents, binders, and lubricants. Other additives which help to give additional desirable physical characteristics to the finished tablet, such as disintegrators, coloring agents, flavoring agents, and sweetening agents might also be added in those compositions. In some embodiments, the solid dosage form is for use as a supplement for an animal (e.g., a dog or cat supplement, a pig supplement, a poultry supplement). In some embodiments, the animal supplement does not contain additional flavoring agents to enhance palatability for the animal.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like.

As used herein, “binders” are agents used to impart cohesive qualities to the powdered material. Binders, or “granulators” as they are sometimes known, impart cohesiveness to the tablet formulation, which insures the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils.

As used herein, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

As used herein, “coloring agents” are agents that give tablets a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye.

As used herein, “flavoring agents” vary considerably in their chemical structure, ranging from simple esters, alcohols, and aldehydes to carbohydrates and complex volatile oils. Natural and synthetic flavors of almost any desired type are now available.

Further materials as well as formulation processing techniques and the like are set out in The Science and Practice of Pharmacy (Remington: The Science & Practice of Pharmacy), 22nd Edition, 2012, Lloyd, Ed. Allen, Pharmaceutical Press, which is incorporated herein by reference.

The compositions of the invention can be in the forms of a powder or syrups. In some embodiments, the compositions of the invention may be in the form of a slurry, syrup, or liquid.

As used herein, “powders” means a solid dosage form intended to be suspended or dissolved in water or another liquid or mixed with soft foods prior to administration. Powders are typically prepared by spray drying or freeze drying of liquid formulations. In some embodiments, the powder is prepared by spray drying. Powders are advantageous due to flexibility, stability, rapid effect, and ease of administration. As used herein, a “slurry” refers to a liquid having the composition contained or suspended therein. In some embodiments, the slurry may comprise a non-aqueous solvent containing the composition. In some embodiments, the slurry may comprise a volume of an aqueous solvent and more of the composition described herein than is dissolvable in the volume of aqueous solvent.

According to a particular aspect, the compositions according to the present invention are useful for use in infant food formulations or in premixes (which are then used to produce infant food formulations). The premix is usually in a dry form. The premix is usually produced by mixing the composition according to the present invention with other suitable ingredients, which are useful and/or essential in an infant formulation and/or premix (or which are useful and/or essential for the production of an infant formulation and/or premix).

According to a particular aspect, an infant formulation in the context of the present invention is usually a dry formulation, which is then dissolved either in water or in milk.

The infant food premix or food formulations may further contain auxiliary agents, for example antioxidants (such as ascorbic acid or salts thereof, tocopherols (synthetic or natural); butylated hydroxytoluene (BHT); butylated hydroxyanisole (BHA); propyl gallate; tert-butyl hydroxyquinoline and/or ascorbic acid esters of a fatty acid); ethoxyquin, plasticizers, stabilizers (such as soy lecithin, citric acid esters of mono- and di-glycerides, and the like), humectants (such as glycerine, sorbitol, polyethylene glycol), dyes, fragrances, fillers and buffers.

According to a further aspect of the present invention, is provided an infant formula comprising a composition of glycoprotein- and glycopeptide-bound oligosaccharides as defined herein for use in promoting, assisting or achieving balanced growth or development in an infant or preventing or reducing the risk of unbalanced growth or development in an infant.

According to a particular aspect of the present invention, an infant formula may further comprise proteins fulfilling the minimum requirements for essential amino acid content and satisfactory growth, for example where over 50% by weight of the protein source is whey. Protein sources based on whey, casein and mixtures thereof may be used as well as protein sources based on soy. As far as whey proteins are concerned, the protein source may be based on acid whey or sweet whey (as readily available by-product of cheese making, preferably where caseino-glyco-macropeptide (CGMP) has been removed) or mixtures thereof and may include alpha-lactalbumin and beta-lactoglobulin in whatever desired proportions.

According to a particular aspect of the present invention, an infant formula may further comprise a carbohydrate source such as lactose, saccharose, maltodextrin, starch and mixtures thereof.

According to a particular aspect of the present invention, an infant formula may further comprise human milk oligosaccharides (HMOs).

According to a particular aspect of the present invention, an infant formula may further comprise a source of lipids including high oleic sunflower oil and high oleic safflower oil. The essential fatty acids linoleic and [alpha]-linolenic acid may also be added as may small amounts of oils containing high quantities of preformed arachidonic acid and docosahexaenoic acid such as fish oils or microbial oils. An infant formula may also contain all vitamins and minerals understood to be essential in the daily diet and in nutritionally significant amounts. Minimum requirements have been established for certain vitamins and minerals. Examples of minerals, vitamins and other nutrients optionally present in the infant formula include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. An infant formula may optionally contain other substances which may have a beneficial effect such as fibres, lactoferrin, nucleotides, nucleosides, and the like.

According to a particular aspect, the animal food formulation according to the invention can be of any form, such as dry product, semi moist product, wet food product or a liquid and includes any food supplement, snack or treat. This includes, standard food products including liquids, as well as pet food treat (for example, snack bars, pet chew, crunchy treat, cereal bars, snacks, biscuits and sweet products). Preferably, the pet foodstuff may be in the form of a dry foodstuff or wet foodstuff. The foodstuff of the first aspect of the invention is, in particular, a nutritionally balanced food product and/or food supplement, for example a pet product and/or pet supplement.

According to a further aspect of the present invention, is provided an animal feed (e.g., a dog food or a cat food) comprising a composition of glycoprotein- and glycopeptide-bound oligosaccharides as defined herein for use in promoting, assisting or achieving balanced growth or development in an animal or preventing or reducing the risk of unbalanced growth or development in an animal. In some embodiments, the animal feed is a pig feed or a poultry feed.

According to a particular embodiment, the animal food formulations or premixes may include one or more nutrients selected from essential amino acids (such as aspartic acid, serine, glutamic acid, glycine, alanine or proline) and essential lipids (such as myristic acid, palmitic acid, stearic acid, palmitoleic acid, oleic acid or linolenic acid).

In a further aspect of the invention, there is provided pet foodstuff comprising the compositions described herein. In some embodiments, the pet foodstuff comprises about 0.5% (w/w), about 0.6% (w/w), about 0.7% (w/w), about 0.8% (w/w), about 0.9% (w/w), about 1% (w/w), about 1.1% (w/w), about 1.2% (w/w), about 1.3% (w/w), about 1.4% (w/w), about 1.5% (w/w), about 1.6% (w/w), about 1.7% (w/w), about 1.8% (w/w), about 1.9% (w/w), about 2% (w/w), about 2.25% (w/w), about 2.5% (w/w), about 2.75% (w/w), or about 3% (w/w) of the composition of the invention. The pet foodstuff may comprise aspartic acid, serine, glutamic acid, glycine, alanine or proline or any combination thereof and myristic acid, palmitic acid, stearic acid, palmitoleic acid, oleic acid or linolenic acid or any combination thereof.

In some embodiments, the compositions are useful as a pharmaceutical composition to treat a human suffering from obesity, diabetes, cardiometabolic diseases or low-grade inflammation.

Methods of manufacturing the compositions described herein are not limited. In some embodiments, the compositions described herein are obtained by the methods of manufacture also described herein.

### Methods of Manufacturing

Some aspects of the disclosure are directed to a method of manufacturing a composition comprising a mixture of glycopeptides, comprising the following steps a)-d): Step a) providing gastrointestinal tract mucins or a partially purified fraction thereof having a pH of approximately 5.0 to 5.5, Step b) optionally concentrating the mucins of step b) by evaporation, Step c) optionally partially removing substances in the mucins having a diameter of less than about 0.2 μm or less than 0.45 μm by filtration or centrifugation, and Step d) removing substances in the mucins having a diameter of greater than 7 μm by filtration or centrifugation.

In some embodiments, the method of manufacturing a composition comprising a mixture of glycopeptides, comprises the following steps a)-d): Step a) providing gastrointestinal tract mucins or a partially purified fraction thereof having a pH of approximately 5.5, Step b) optionally concentrating the mucins of step b) by evaporation, Step c) partially removing substances in the mucins having a diameter of less than about 0.2 μm or less than 0.45 μm by filtration or centrifugation, and Step d) removing substances in the mucins having a diameter of greater than 7 μm by filtration or centrifugation.

As used herein, the gastrointestinal tract mucins or a partially purified fraction thereof having a pH of approximately 5.0-5.5 set forth in step a) comprise any gastrointestinal tract mucins described herein. In some embodiments, a partially purified fraction thereof comprises hydrolyzed gastrointestinal tract mucins as described herein (e.g., from a waste stream of an industrial process). In some embodiments, the gastrointestinal tract mucins or a partially purified fraction thereof have been treated with a base (e.g., sodium hydroxide) in order to obtain a pH of 5.0-5.5.

In some embodiments, the mucins of step a) are purified to remove large insoluble particles, lipids, and fats. In some embodiments, the mucins were purified by centrifugation at 500 to 10,000×g and the supernatant collected to remove large insoluble particles, lipids, and fats. In some embodiments of step b), the mucins were passed through a filter having a cut-off of about 100 kDa and the filtrate collected to remove large insoluble particles, lipids, and fats.

In some embodiments of step b), the mucins are concentrated by partial evaporation with, e.g., a rotary evaporator. In some embodiments, evaporation reduces the total purified mucin volume by about 10%, 20%, 30%, 40%, 50%, 60%, 70%, 80%, 85%, 90%, 95%, or more. In some embodiments, the mucins are concentrated by filtration. For example, the mucins may be filtered to remove excess water, optionally with alcohol (e.g., n-butanol) and water washing steps. In some embodiments, the filtration is with a 0.45 μm filter.

In some embodiments of step c), partially removing substances in the mucins having a diameter of less than about 0.45 μm comprises partial filtration with a cut-off of 0.45 μm.

In some embodiments, step a) further comprises desalinating the mucins. In some embodiments, the mucins are desalinated with a desalting column. Desalting columns are known in the art and not limited. In some embodiments, the desalting column has a resin with an exclusion limit or molecular weight cut off (MWCO) of between 5 and 10 kDa. In some embodiments, the mucins are desalinated by dialysis with an appropriate buffer and a dialysis membrane blocking movement of amino acids, proteins, or glycans across the membrane.

In some embodiments, step d) comprises filtration of the mucins by passage through Whatman paper and collection of the filtrate. Methods of removing substances of a certain size are known in the art and are not limited.

Some embodiments of the methods of manufacture disclosed herein further comprise a step e) of further purifying the mucins by ultrafiltration, thereby removing particles and molecules having a weight of less than about 5 kDa, 3 kDa, 2 kDa, or 1 kDa.

Some embodiments of the methods of manufacture disclosed herein further comprise a step f) of drying the resultant composition comprising a mixture of glycopeptides. Methods of drying the composition are known in the art and are not limited. In some embodiments, the composition is dried with a roto-evaporator. In some embodiments, the composition is dried via spray drying. In some embodiments, the spray drying results in particles having a range of about 10 to 150 μm.

Some embodiments of the methods of manufacture disclosed herein further comprise a step g) of adding the composition to a foodstuff. In some embodiments, the composition is added to the foodstuff after step e) described above (e.g., the composition is added as a liquid, slurry or syrup). In some embodiments, the composition is added to the foodstuff after step f) described above (e.g., the composition is added as a powder or solid). In some embodiments, the foodstuff is an animal feed (e.g., dog food, dog treat, cat food, cat treat). In some embodiments, the foodstuff is a pig feed or poultry feed. In some embodiments, the composition is added to the foodstuff to a final amount of 0.5% to 2.0% w/w.

In some embodiments of the methods disclosed herein, the resulting composition comprising a mixture of glycopeptides has a water solubility of 80-120 g/L at 25° C. In some embodiments, the composition has a water solubility of about 80 g/L, about 85 g/L, about 90 g/L, about 95 g/L, about 100 g/L, about 105 g/L, about 110 g/L, about 115 g/L, or about 120 g/L at 25° C. In some embodiments, the composition has a water solubility of about 120 g/L or more at 25° C.

In some embodiments of the methods disclosed herein, the oligosaccharide content of the resulting composition comprising a mixture of glycopeptides is >5% (w/w). In some embodiments, the oligosaccharide content of the composition is greater than about 1.8% (w/w), greater than about 2.0% (w/w), greater than about 2.5% (w/w), greater than about 3% (w/w), greater than about 5% (w/w), greater than about 10% (w/w), greater than about 11% (w/w), greater than about 12% (w/w), greater than about 15% (w/w), greater than about 20% (w/w), or more.

In some embodiments, the resulting composition comprises glycoprotein- or glycopeptide-bound oligosaccharides having at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or all of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6 SGlcNAcβ1-6)GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3 [(6S)GlcNAcβ1-6]GalNAc, Galβ1-3(Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-) GlcNAcβ1-6]GalNAc, and Fucα1-2Galβ1-3 [Fucα1-2Galβ1-4(6 S)GlcNAcβ1-6]GalNAc. Methods of determining the structure of oligosaccharides bound to glycoproteins and glycopeptides are known in the art and are not limited. In some embodiments, the structure of oligosaccharides bound to glycoproteins and glycopeptides is determined by tandem mass spectrometry (MS/MS).

In some embodiments, the resulting composition comprises glycopeptide-bound oligosaccharides having at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, or all of the following structures: Galβ1-3GalNAc, GlcNAcβ1-6GalNAc, NeuAcα2-6GalNAc, NeuGcα2-6GalNAc, Fucα1-2Galβ1-3GalNAc, Gal+GlcNAcβ1-6GalNAc, Galβ1-3(GlcNAcβ1-6)GalNAc, Galβ1-3GlcNAcβ1-6GalNAc, Galβ1-3(GlcNAcβ1-6)GalNAc, Galβ1-3(6SGlcNAcβ1-6)GalNAc, Galβ1-3(NeuAcα2-6)GalNAc, NeuAcαα2-3Galβ1-3GalNAc, Galβ1-3(NeuGcα2-6)GalNAc, NeuGcα2-3Galβ1-3GalNAc, GlcNAc-(NeuAcα2-6)GalNAc, GalNAc-(NeuAcα2-6)GalNAc, HexNAc-(NeuGcα2-6)GalNAc, Fucα1-2(GalNAcα1-3)Galβ1-3GalNAc, Fucα1-2Galβ1-4GlcNAcβ1-6GalNAc, Fucα1-2Galβ1-3(GlcNAcβ1-6)GalNAc, Fucα1-2Galβ1-3(6S-GlcNAcβ1-6)GalNAc, Fucα1-2Galβ1-3(NeuAcβ2-6)GalNAc, GlcNAcβ1-3[Galβ1-4(6S)GlcNAcβ1-6]GalNAc, Galβ1-4GlcNAcβ1-3[(6S)GlcNAcβ1-6]GalNAc, Galβ1-3 (Fucα1-2Galβ1-4GlcNAcβ1-6)GalNAc, Fucα1-2Galβ1-4(6S)GlcNAcβ1-6[GlcNAcβ1-3]GalNAc, GlcNAcβ1-3[Fucα1-2Galβ1-3(6S-)GlcNAcβ1-6]GalNAc, and Fucα1-2Galβ1-3[Fucα1-2Galβ1-4(6S)GlcNAcβ1-6]GalNAc. Methods of determining the structure of oligosaccharides bound to glycopeptides are known in the art and are not limited. In some embodiments, the structure of oligosaccharides bound to glycopeptides is determined by tandem mass spectrometry (MS/MS).

In some embodiments, the resulting composition comprises glycoprotein- or glycopeptide-bound oligosaccharides, or glycopeptide-bound oligosaccharides, having at least 14 of the structures shown above. In some embodiments, the resulting composition comprises glycoprotein- or glycopeptide-bound oligosaccharides, or glycopeptide-bound oligosaccharides, having at least 21 of the structures shown above. In some embodiments, the resulting composition comprises glycoprotein- or glycopeptide-bound oligosaccharide, or glycopeptide-bound oligosaccharides, having each of the structures shown above. In some embodiments, the resulting composition comprises glycoprotein- or glycopeptide-bound oligosaccharides having at least 20, 21, 22, 23, 24, 25, 26, 27, 28, 29, 30, 31, 32, 33, 34, 35, 36, 37, 38, 39, or 40 different oligosaccharide structures.

In some embodiments, the resulting composition comprising a mixture of glycopeptides comprises less than 1% free glycans (w/w). In some embodiments, the resulting composition comprises less than about 5%, less than about 2.5%, less than about 1%, less than about 0.5%, less than about 0.1%, or less than about 0.01% free glycans (w/w). In some embodiments, the resulting composition comprising a mixture of glycopeptides comprises substantially no glycans. Methods of measuring free glycans are known in the art and are not limited. In some embodiments, free glycans are measured by LC-MS/MS.

In some embodiments, the partially purified fraction of mucins of step a) has been partially depleted of glycans by enzymatic hydrolysis. In some embodiments, the mucins of step a) have been hydrolyzed. In some embodiments, the gastrointestinal tract mucins are porcine gastrointestinal tract mucins. In some embodiments, the gastrointestinal tract mucins are porcine gastrointestinal tract mucins from an industrial waste stream.

In some embodiments, the obtained composition comprising a mixture of glycopeptides causes reduced growth of Escherichia coli when orally administered to a subject than a composition derived from the same process but not purified to remove insoluble particles greater than 7 μm. The type of Escherichia coli is not limited. In some embodiments, the Escherichia coli is commensal Escherichia coli. In some embodiments, the Escherichia coli is pathogenic Escherichia coli (e.g., associated with diarrheal diseases). In some embodiments, the Escherichia coli is both commensurate and pathogenic Escherichia coli. In some embodiments, “reduced growth of Escherichia coli” means at least about 10%, at least about 20%, at least about 30%, at least about 40%, at least about 50%, at least about 60%, at least about 70%, at least about 80%, at least about 90%, or at least about 95% less growth of Escherichia coli.

In some embodiments, the obtained composition comprising a mixture of glycopeptides causes increased growth of Akkermansia muciniphila gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Bifidobacterium bifidum gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Lactobacillus acidophilus gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Bifidobacterium animalis subsp. lactis gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Bifidobacterium breve gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Bacteroides thetaiotaomicron gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Coprococcus comes gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Prevotella copri gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Bacteroides vulgatus gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the obtained composition comprising a mixture of glycopeptides causes increased growth of Megamonas spp. gut microbiota when orally administered to a subject. In some embodiments, growth is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

In some embodiments, the resulting or obtained composition comprising a mixture of glycopeptides causes more growth of commensal bacteria when orally administered to a subject than a composition (e.g., an equivalent composition) treated to comprise a mixture of free glycans instead of a mixture of glycopeptides. In some embodiments, the one or more commensal bacteria comprise Coprococcus comes, Prevotella copri, Megamonas spp., or Bacteroides vulgatus. In some embodiments, growth is at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold more than growth caused by administration of an equivalent composition further treated to comprise a mixture of free glycans instead of a mixture of glycopeptides.

In some embodiments, the obtained composition comprising a mixture of glycopeptides causes increased production of SCFA (e.g., butyrate and/or propionate production) in the gut when orally administered to a subject. In some embodiments, production is increased by at least about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, or 5-fold.

### Methods of Treatment

The present compositions comprise mixtures of glycoprotein- or glycopeptide-bound oligosaccharides, or only glycopeptide-bound oligosaccharides, that are much more structurally diverse than previous pre-biotic formulations, in particular prebiotics containing fructooligosaccharides (FOS) and/or galactoligosaccharides (GOS). FOS and GOS are linear chain, simpler oligosaccharides that do not contain the structural complexity and diversity of the present composition. Specifically, the present composition comprises branched structures containing fucose, sialic acid, and N-acetylglucosamine, which are completely absent in FOS and GOS. Further, some of the oligosaccharides in the present composition are sialylated while GOS and FOS do not contain any sialic acid at all. Thus, unlike these previous prebiotics, the glycoprotein- or glycopeptide-bound oligosaccharides, or only glycopeptide-bound oligosaccharides, of the present composition have multiple building blocks, branched structures and a higher variety of structures which impart biological functionalities including anti-microbial activity, better microbiota maintenance, and immunological activity.

Some aspects of the present invention are related to a method of treating, preventing, or reducing the severity of a pathogenic microorganism infection of the gut of a subject comprising orally administering to the subject a composition disclosed herein or a composition manufactured by a method disclosed herein. In some embodiments, the pathogenic microorganism is selected from Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition inhibits glycan-mediated binding of one or more pathogenic micro-organisms to mucosal cells by about 5%, 10%, 20%, 30%, 40%, 50%, 60%, 70%, 80%, 85%, 90%, 95%, 99%, 99.9% or more. In some embodiments, administration of the composition inhibits glycan-mediated binding of one or more pathogenic micro-organisms to mucosal cells by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, administration of the composition to a patient inhibits growth or decreases the level of one or more pathogenic microorganisms (e.g., Escherichia coli) in the gut of the patient by about 5%, 10%, 20%, 30%, 40%, 50%, 60%, 70%, 80%, 85%, 90%, 95%, 99%, 99.9% or more. In some embodiments, administration of the composition to a patient inhibits growth or decreases the level of one or more pathogenic microorganisms (e.g., pathogenic Escherichia coli) in the gut of the patient by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold or more.

Some aspects of the present invention are related to a method of reducing the fat mass of a subject comprising orally administering to the subject a composition disclosed herein or a composition manufactured by a method disclosed herein.

Some aspects of the present invention are related to a method of treating, preventing, or reducing inflammation in a subject comprising orally administering to the subject a composition disclosed herein or a composition manufactured by a method disclosed herein. In some embodiments, administration of the composition reduces inflammation (e.g., inflammation in the gut) by about 5%, 10%, 20%, 30%, 40%, 50%, 60%, 70%, 80%, 85%, 90%, 95%, 99%, 99.9% or more. In some embodiments, administration of the composition reduces inflammation (e.g., inflammation in the gut) by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, reduces a level of calprotectin in the blood stream or stool of the subject. In some embodiments, calprotectin is decreased in the stool or decreased in the blood by about 5%, 10%, 20%, 30%, 40%, 50%, 60%, 70%, 80%, 85%, 90%, 95%, 99%, 99.9% or more. In some embodiments, calprotectin is decreased in the stool or blood by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more (e.g., compared to before administration of a composition of the invention).

Some aspects of the present invention are related to a method of increasing production of short chain fatty acid (SCFA) (e.g., butyrate and/or propionate) in the gut of a subject comprising orally administering to the subject a composition disclosed herein or a composition manufactured by a method disclosed herein. In some embodiments, SCFA production is increased by about 5%, 10%, 20%, 30%, 40%, 50%, 60%, 70%, 80%, 85%, 90%, 95%, 99%, 99.9% or more. In some embodiments, SCFA production is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, the composition, when orally administered to a subject, is capable of lowering pH in the gut of the subject. In some embodiments, the decrease in pH is caused by an increase in SCFA production in the gut.

In some embodiments, administration of the composition to a patient increases growth or increases the level of one or more commensal bacteria (e.g., Coprococcus comes, Prevotella copri, Megamonas spp., and/or Bacteroides vulgatus) in the gut of the patient by about 5%, 10%, 20%, 30%, 40%, 50%, 60%, 70%, 80%, 85%, 90%, 95%, 99%, 99.9% or more. In some embodiments, administration of the composition to a patient increases growth or increases the level of one or more commensal bacteria (e.g., Coprococcus comes, Prevotella copri, Megamonas spp., and/or Bacteroides vulgatus) in the gut of the patient by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold or more.

Some aspects of the present invention are related to a method of improving gut barrier integrity in the gut of a subject comprising orally administering to the subject a composition disclosed herein or a composition manufactured by a method disclosed herein.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

### EXAMPLES

### Example 1—Preparation and Analysis of GNU100 (Prep #1)

Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (500 to 10,000×g) to remove large insoluble particles, fats, and lipids. The mucins were then desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further processed by partially filtering through a 0.2 μM Polyethersulfone (PES) filter (Millipore Sigma) to remove some amino acids and salts, and the retentate collected.

One hundred ml of the retentate was filtrated on Whatman filter paper (diameter 110 mm, pore size 4-7 inn) by suction using a Büchner funnel. About 100 ml of filtrate (brown liquid) was obtained. The solid residue was discarded, and the filtrate dried under rotary evaporator at 50 mbar and 50° C. m=31.8 g. Total yield=31.8%. Dry substance yield=88% to yield a powder composition of the claimed invention labeled GNU100. The powder composition was white to yellow with a neutral or slight amino acid smell and had a 2-5% moisture content. The water solubility of the powder was 80 to 120 g/L at 25° C.

Analysis of glycan content of GNU100-O-glycans were released from glycopeptides and glycoproteins in GNU100 by β-elimination in 50 mM NaOH and 0.5M NaBH4. If needed, pH was adjusted to above 12, which is required for a successful release reaction. The samples were incubated in 50° C., with the lids loosely tightened. On day 2, the samples were slowly neutralized with concentrated acetic acid (HAc). Aliquots (20 ul) of the samples were desalted using cation exchange resin (AG50W×8) packed onto a ZipTip C18 tip. After drying the samples in SpeedVac, 50 ul 1% Acetic Acid (HAc) in methanol was added five times to remove residual borate by evaporation.

Released glycans were resuspended in water and analyzed by liquid chromatograph-electrospray ionization tandem mass spectrometry (LC-ESI/MS). The oligosaccharides were separated on a column (10 cm×250 (μm) packed in-house with 3 (μm porous graphite particles (Hypercarb, Thermo-Hypersil, Runcorn, UK). The oligosaccharides were injected on to the column and eluted with an acetonitrile gradient (Buffer A, 10 mM ammonium bicarbonate; Buffer B, 10 mM ammonium bicarbonate in 80% acetonitrile); Buffer C: 0.1% HAc. The gradient (0-45% Buffer B) was eluted for 30 min, followed by 8 min with 100% Buffer B, followed by 10 min with 0.1% HAc, and equilibrated with Buffer A in the next 15 min. A 40 cm×50 (μm i.d. fused silica capillary was used as transfer line to the ion source.

The samples were analyzed in negative ion mode on a LTQ linear ion trap mass spectrometer (Thermo Electron, San José, CA), with an IonMax standard ESI source equipped with a stainless steel needle kept at −3.5 kV. Compressed air was used as nebulizer gas. The heated capillary was kept at 270° C., and the capillary voltage was −50 kV. Full scan (m/z 380-2000, two microscan, maximum 100 ms, target value of 30,000) was performed, followed by data-dependent MS² scans (two microscans, maximum 100 ms, target value of 10,000) with normalized collision energy of 35%, isolation window of 2.5 units, activation q=0.25 and activation time 30 ms. The threshold for MS² was set to 300 counts. Data acquisition and processing were conducted with Xcalibur software (Version 2.0.7).

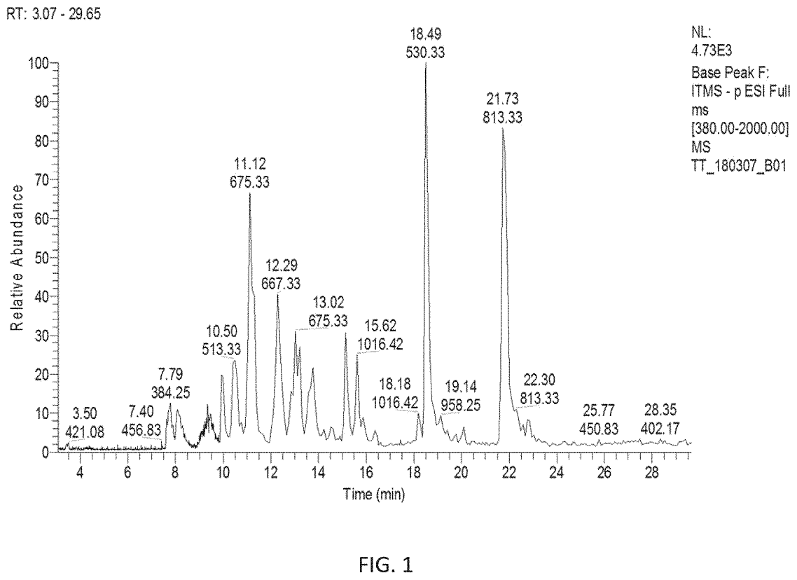

The chromatogram resulting from this analysis is shown in FIG. 1 wherein the values on top of each peak indicate the retention time and m/z value, respectively. The general formula of the detected glycans, as well as their putative structures are shown in Table 1 below:

TABLE 1
Oligosaccharides structures in GNU100 as obtained via LCS MS.

| Name ^{[1]} | Composition ^{[2]} | Putative structures ^{[3]} | RT ^{[4]} |
|---|---|---|---|
| 384 | Hex1HexNAc1 | Galβ1-3GalNAcol | 7.8 |
| 425 | HexNAc2 | GlcNAcβ1-6GalNAcol | 8.1, 9.9 |
| 513 | NeuAc1HexNAc1 | NeuAcα2-6HalNAcol | 10.5 |
| 529 | NeuGc1HexNAc1 | NeuGcα2-6GalNAcol | 10.3 |
| 530 | Hex1HexNAc1deHex1 | Fucα1-2Galβ1-33GalNacol | 18.5 |
| 587-1 | Hex1HexNAc2 | Gal + GlcNAcβ1-6GalNAcol | 10.1 |
| 587-2 | Hex1HexNAc2 | Galβ1-3(GlcNAcβ1-6)GalNAcol | 10.4 |
| 587-3 | Hex1HexNAc2 | Galβ1-3GlcNAcβ1-6GalNAcol | 11.2 |
| 587-4 | Hex1HexNAc2 | Galβ1-3(GlcNAcβ1-6)GalNAcol | 12.01 |
| 667 | Hex1hexNAc2Sul1 | Galβ1-3(6SGlcNAβ1-6)GalNAcol | 12.3 |
| 675-1 | NeuAc1Hex1HexNAc1 | Galβ1-3(NeuAcα2-6)GalNAcol | 11.1 |
| 675-2 | NeuAc1Hex1HexNAc1 | NeuAcαα2-3Galβ1-3GalNAcol | 13.2 |
| 691-1 | NeuGc1Hex1HexNAc1 | Galβ1-3(NeuAcα2-6)GalNAcol | 11 |
| 691-2 | NeuGc1Hex1HexNAc1 | NeuGcα2-3Galβ1-3GalNAcol | 12.8 |
| 716-1 | NeuAc1HexNAc2 | HexNAc-(NeuAcα2-6)GalNAcol | 11.3 |
| 716-2 | NeuAc1HexNAc2 | HexNAc-(NeuAcα2-6)GalNAcol | 13.8 |
| 732 | NeuGc1HexNAc2 | HexNAc-(NeuGcα2-6)GalNAcol | 11.1 |
| 733-1 | Hex1HexNAc2deHex1 | Fucα1-2(GalNAcα1-3)Galβ1-3GalNAcol | 13.21 |
| 733-2 | Hex1HexNAc2deHex1 | Fucα1-2Galβ1-4GlcNAcβ1-6GalNAcol | 15.4 |
| 733-3 | Hex1HexNAc2deHex1 | Fucα1-2Galβ1-3(GlcNAcβ1-6)GalNAcol | 18.9 |
| 813 | Hex1HexNAc2deHex1Sul1 | Fucα1-2Galβ1-3(6S-GlcNAcβ1-6)GalNAcol | 21.7 |
| 821 | NeuAc1Hex1HexNAc1deHex1 | Fucα1-2Galβ1-3(NeuAcβ2-6)GalNAcol | 21.8 |
| 870-1 | Hex1HexNAc3Sul1 | GlcNAcβ1-3[Galβ1-4(6S)GlcNAcβ1-6]GalNAcol | 14.6 |
| 870-2 | Hex1HexNAc3Sul1 | Galβ1-4GlcNAcβ1-3[(6S)GlcNAcβ1-6]GalNAcol | 15.1 |
| 895-1 | Hex2HexNAc2deHex1 | Galβ1-3(Fucα1-2Galβ1-4GlcNAcβ1-6)GalNAcol | 15.4 |
| 1016-1 | Hex1HexNAc3deHex1Sul1 | Fucα1-2Galβ1-4(6S)GlcNAcβ1-6[GlcNAcβ1-3]GalNAcol | 14.5 |
| 1016-2 | Hex1HexNAc3deHex1Sul1 | GlcNAcβ1-3[Fucα1-2Galβ1-3(6S-)GlcNAcβ1-6]GalNAcol | 18.19 |
| 1121 | Hex2HexNAc2deHex2Sul1 | Fucα1-2Galβ1-3[Fucα1-2Galβ1-4(6S)GlcNAcβ1-6]GalNAcol | 21.8 |

Footnote:
^{[1]} The names of structures;
^{[2]} Hex, hexose; HexNAc, N-acetylhexosamine; deHex, fucose; NeuAc, N-acetylneuraminic acid; NeuGc, N-acetyl glycolylneuraminic acid; S, sulphate
^{[3]} structures are given in the text according following rules: the structure is described clockwise and left-to-right where reducing end locates righmost side (as shown in cartoon figure); “ + ” is used for uncertain location Gal, galactose; Galol, alditol form of Gal; Gal NAc, N-acetylgalactosamine; GalNAcol, alditol form of Gal NAc; GlcNAc, N-acetylglucosamine; Fuc, fucose; S, sulfate; NeuAc/NeuGc, N-acetylneuramnic acid/N-acetyl glycolylneraminic acid
^{[4]} Retention time (RT) of selected structure on LC;
Recitation of “Hex” in structures 716-1 and 716-2 correspond to Glc or Gal

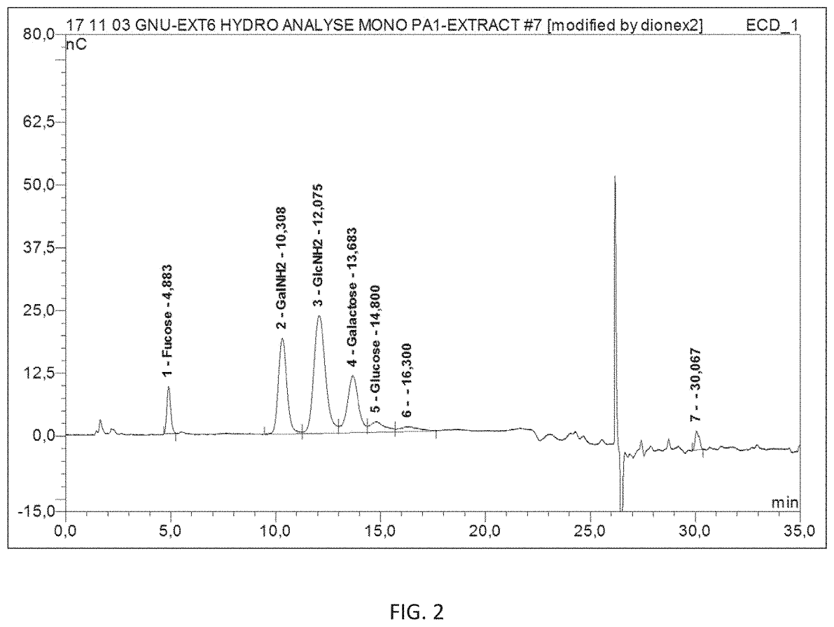

Determination of Principle Sugars in GNU100—HPAEC-PAD (High-performance anion exchange chromatography with derivatization-free, pulsed amperometric detection) was performed on the GNU100 composition to determine the principal sugars in the oligosaccharide component. This result is shown in a chromatogram in FIG. 2.

Specifically, GNU100 was freeze dried to remove water and treated with TFA 2N at 5 g/L at 100° C. during 4 hours under agitation to obtain free monosaccharides. The sample was then neutralized (NaOH 19N), diluted with distilled water and filtered through an 0.24 μm filter. The resulting sample was brought to a concentration of 100 mg/L to 500 mg/L of monosaccharides and loaded on a CarboPac PA-1 (Dionex) 4×250 mm analytical column to perform HPAEC-PAD with the following parameters.
System: ICS 2500 (Dionex) with pump, electrochemical detector, thermal compartment and autosampler.
Temperature of column 17° C.
Rate of elution: 1 mL/min
Volume of sample: 20 μl
Detection: Electrochemical detection PAD with reference electrode mode Ag/Cl.
Data Acquisition Software: Chromeleon (Dionex).

Elution Gradient: NaOH from 0.18 mM to 200 mM; Sodium Acetate from 0 to 500 mM. A mixture of external standards of monosaccharides (Fuc, GalNH2, GlcNH2, Gal, Glc at 6 mg/L and 12 mg/L) was analyzed in parallel to identify and quantify each monosaccharide in the tested sample.

Based on the results of the HPAEC-PAD analysis, the principle composition and content of monosaccharides in GNU100 were determined, as shown in Table 2.

TABLE 2
Composition and content of monosaccharides in GNU 100.
Oligosaccharides quantitative composition in mg/L for 100 mg/L

| Sam- | GalNac- | | | | Galac- | | |
|---|---|---|---|---|---|---|---|
| ple | ol | Fucose | GalNH2 | GlcNH2 | tose | Glucose | Total |
| GNU | 0 | 0.169 | 0.696 | 0.915 | 0.282 | 0 | 2.062 |
| 100 | | | | | | | |

Free Amino Acids analysis of GNU100—GNU100 was dissolved in water to obtain 200 mg/ml solution. 25 μL of prepared solution was extracted with 275 μL of pre-cooled Acetonitrile (ACN):H2O (5:1, v/v) solvent containing internal standards. This solvent and sample mixture was vortexed and incubated for 1 hour at −20° C., followed by 15 min centrifugation (at 13,000 rpm at 4° C.) to facilitate protein precipitation. The resulting supernatant was collected and analyzed using Hydrophilic Interaction Liquid Chromatography coupled to High Resolution Mass Spectrometry (HILIC-HRMS) in positive ionization mode on a Q Exactive™ Hybrid Quadrupole-Orbitrap interfaced with Thermo Accela 1250 UPLC pump and CTC PAL Analytics autosampler. Amino acids were separated using a BEH Amide, 1.7 μm, 100 mm×2.1 mm I.D. column (Waters, Massachusetts, US). The mobile phase was composed of A=10 mM ammonium formate and 0.1% FA in water and B=0.1% FA in ACN. The instrument was set to acquire over the m/z range 60-900 at 70′000 FWHM resolution.

Amino acids and derivatives were quantified by using a standard calibration curves and isotopic labeled internal standards (please Table 3 below). Data was processed using TraceFinder Clinical Research (version 4.1, Thermo Fischer Scientific).

TABLE 3
List of quantified amino acids with the concentration
range of a calibration curve and stable isotope
labeled standard used for each acid.

| Amino acids & | Stable isotope- | Concentration |
|---|---|---|
| derivatives | labeled standard | range (μM) |
| 2-Aminoadipate | Tyrosine (13C9, 15N) | 4-500 |
| Alanine | Alanine (13C3, 15N) | 20-2500 |
| alpha-Aminobutyrate | Tyrosine (13C9, 15N) | 4-500 |
| Asparagine | Asparagine (13C4) | 2-250 |
| Aspartate | Aspartate (13C4, 15N) | 1-124 |
| beta-Alanine/Sarcosine | Alanine (13C3, 15N) | 4-508 |
| Cirtulline | Cirtulline (Ureido-13C) | 8-1000 |
| Creatine | Alanine (13C3, 15N) | 2-255 |
| Creatinine | Phenylalanine (13C3, 15N) | 31-4000 |
| Guanidinoacetate | Threonine (13C4, 15N) | 4-500 |
| gamma-Aminobutyrate | Methionine (13C5, 15N) | 4-495 |
| Glutamine | Glutamate (13C5, 15N) | 39-5000 |
| Glutamate | Glutamate (13C5, 15N) | 10-1248 |
| Histidine | Histidine (13C6; 15N3) | 8-1025 |
| Hydroxyproline | Proline (13C5, 15N) | 2-300 |
| Isoleucine/Allo Isoleucine | Isoleucine (13C6, 15N) | 8-1000 |
| Kynurenine | Phenylalanine (13C3, 15N) | 4-500 |
| Leucine | Leucine (13C6, 15N) | 8-1000 |
| Methionine | Methionine (13C5, 15N) | 2-263 |
| Phenylalanine | Phenylalanine (13C3, 15N) | 2-259 |
| Pipecolate | Tyrosine (13C9, 15N) | 4-500 |
| Proline | Proline (13C5, 15N) | 8-1010 |
| Taurine | Taurine (1,2-13C2) | 39-5010 |
| Threonine | Threonine (13C4, 15N) | 5-579 |
| Tryptophan | Phenylalanine (13C3, 15N) | 2-260 |
| Tyrosine | Tyrosine (13C9, 15N) | 2-246 |
| Valine | Valine (13C5, 15N) | 8-1010 |

Elemental Analysis of GNU100 An elemental analysis of the GNU100 sample was also performed. Carbon, hydrogen and nitrogen content were determined with a CHN analyzer (PerkinElmer). Chlorine content was determined with a FX Amperometric Total Chlorine Analyzer (FoxCroft). Sulfur, phosphorus, boron and sodium were measured with a Thermo Fisher Scientific ICP-iCAP 7400 elemental analyzer. Finally, fluoride content was determined by mineralizing the sample via the Wurzchmitt method followed by using the TISAB IV reagent and a fluoride ion selective electrode (Thermo Fisher Scientific).

The results are shown in Table 4.

TABLE 4

| Elements | GNU100 |
|---|---|
| C (%) | 31.2 |
| H (%) | 7.6 |
| N (%) | 9.9 |
| B (ppm) | <1 |
| Cl tot. (ppm) | 2000 |
| F (ppm) | <500 |
| Na (ppm) | 44000 |
| P (ppm) | 11000 |
| S tot. (ppm) | 2000 |
| As (ppm) | <1 |
| Cd (ppm) | <1 |
| Pb (ppm) | <1 |
| Hg (ppm) | <1 |

### Example 2. Alternate Preparation of GNU100 (Prep #2)

Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (500 to 10,000×g) to remove large insoluble particles, fats, and lipids. The mucins were desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further processed by partially filtering through a 0.2 μM Polyethersulfone (PES) filter (Millipore Sigma) to remove some salts and amino acids, and the retentate collected.

One hundred ml of the retentate was filtrated on Whatman filter paper (diameter 110 mm, pore size 4-7 μm) by suction using a Büchner funnel. 100 ml of filtrate (brown liquid) was obtained. The filtrate was then ultra-filtrated though a PES membrane having a molecular weight cutoff (MWCO) of 2 kDa (Millipore Sigma) and the retentate collected. The retentate was then dried under rotary evaporator at 50 mbar and 50° C., to yield a powder composition of the claimed invention having substantially the same properties as the GNU100 of Example 1. However, the powder of Example 2 exhibited reduced growth of Escherichia coli in in vitro bacterial culture as compared to the powder of Example 1.

### Example 3 Alternate Preparation of GNU100 (Prep #3)

Partially purified hydrolyzed porcine gastrointestinal tract mucins were obtained from commercial sources and stabilized at a pH of 5.0 using sulfuric acid or sodium hydroxide, as appropriate. The stabilized mucins were then centrifuged at low speed (5,000 to 10,000×g) to remove large insoluble particles and lipids. The mucins were then desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further purified by filtering through a 0.2 μM Polyethersulfone (PES) filter (Millipore Sigma) and the retentate collected.

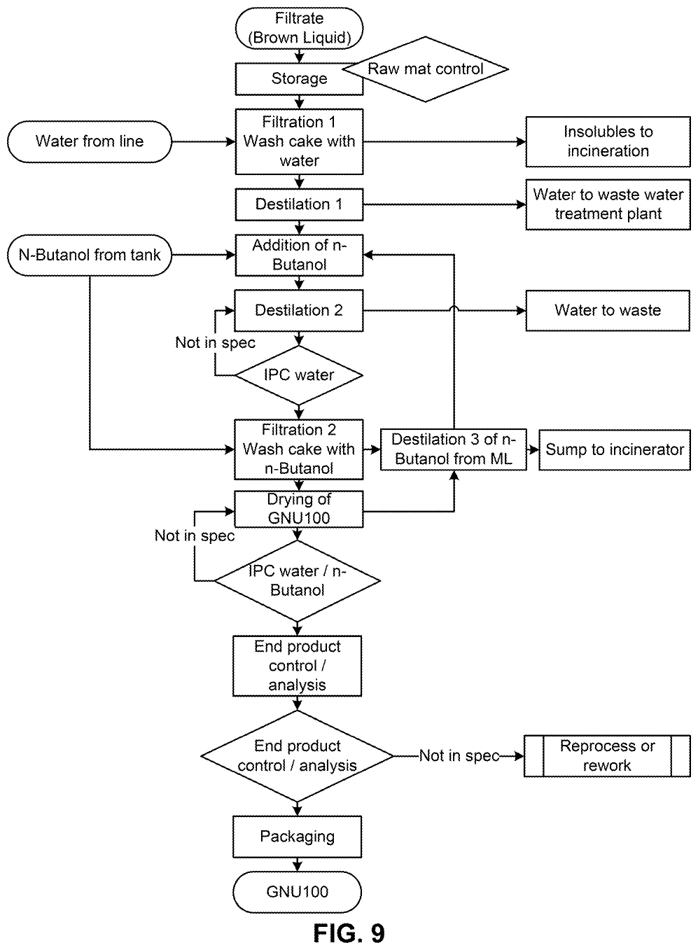

One hundred ml of the retentate was filtrated on Whatman filter paper (diameter 110 mm, pore size 4-7 inn) by suction using a Büchner funnel. 100 ml of filtrate (brown liquid) was obtained. The filtrate was then concentrated by the process shown in FIG. 9, producing a composition of the claimed invention having substantially the same properties as the GNU100 of Example 1.

### Example 4 Bacterial Growth in GNU100 Supplemented Media

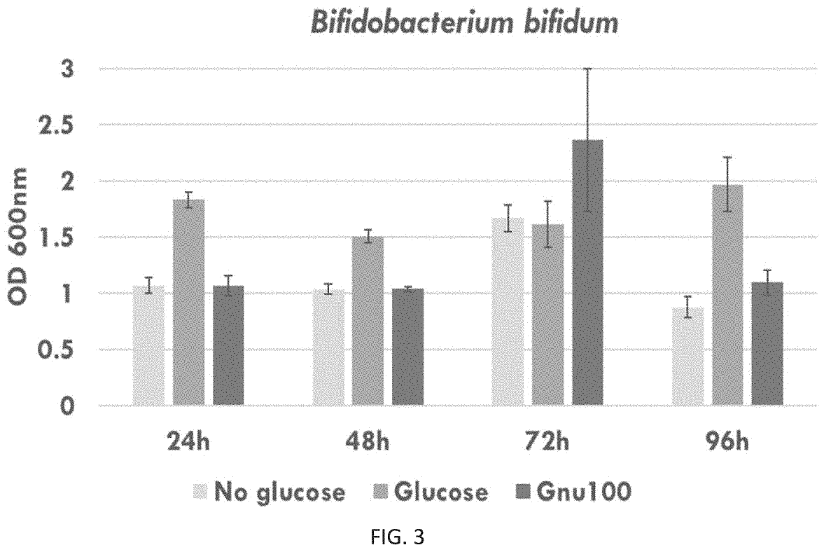

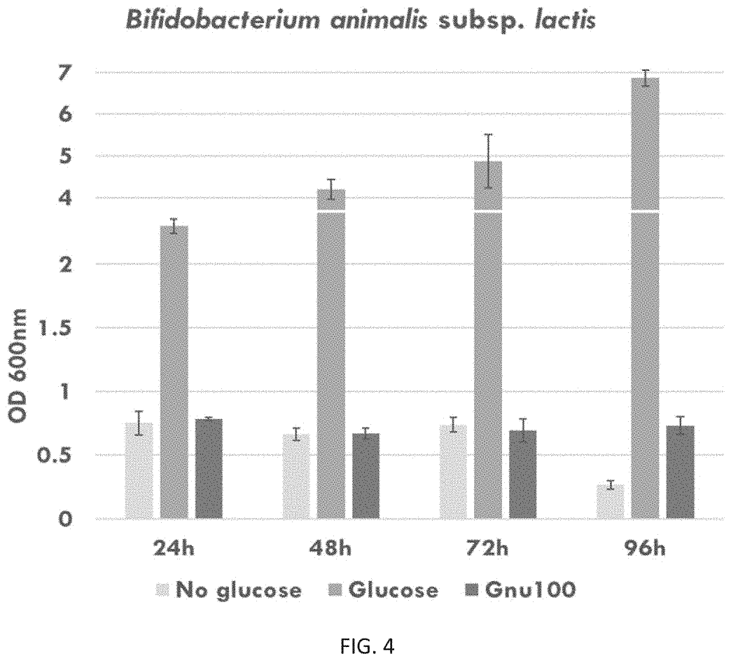

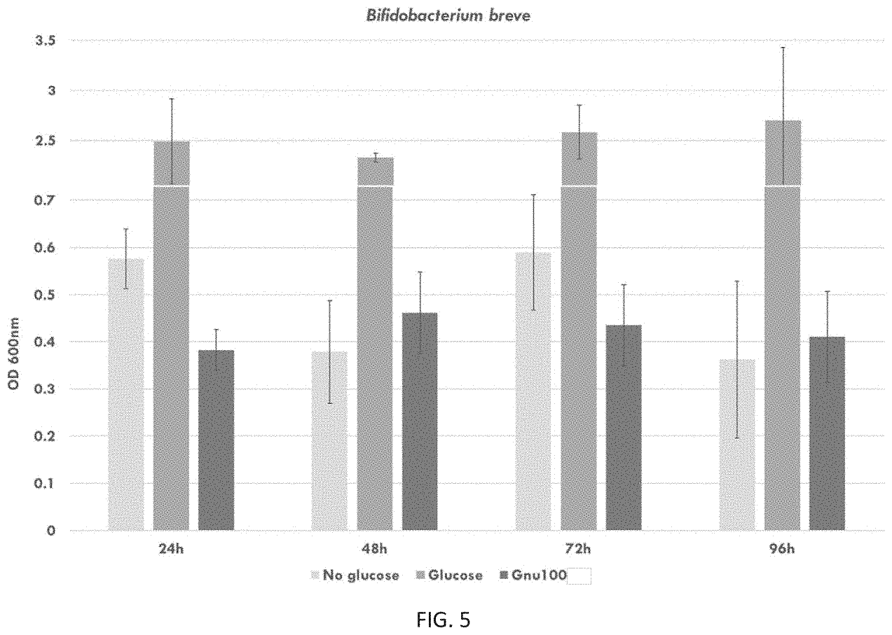

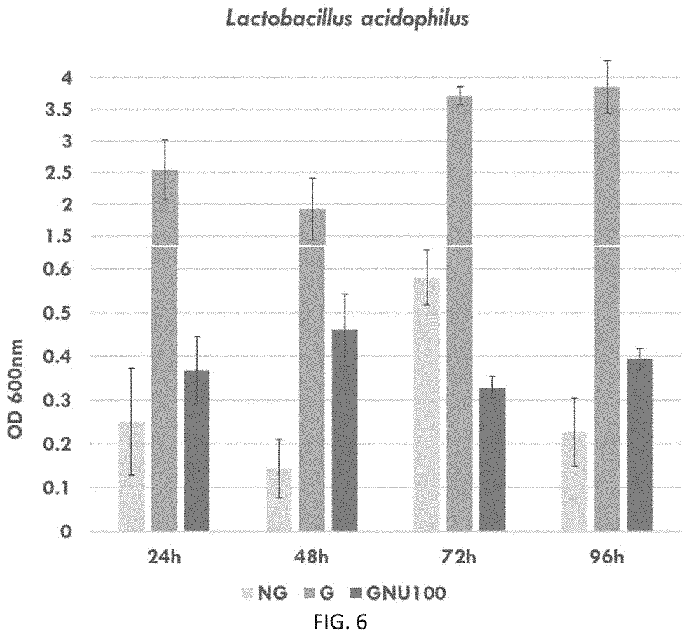

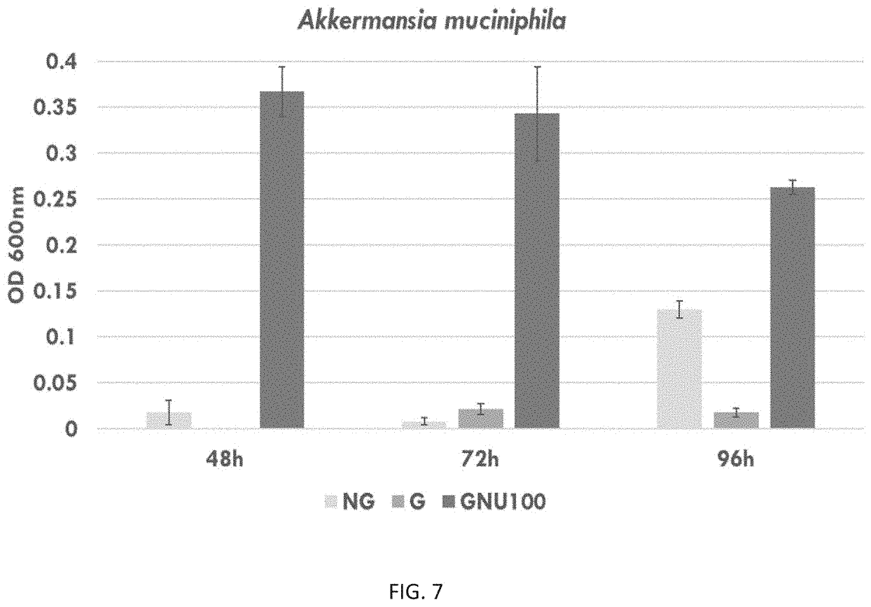

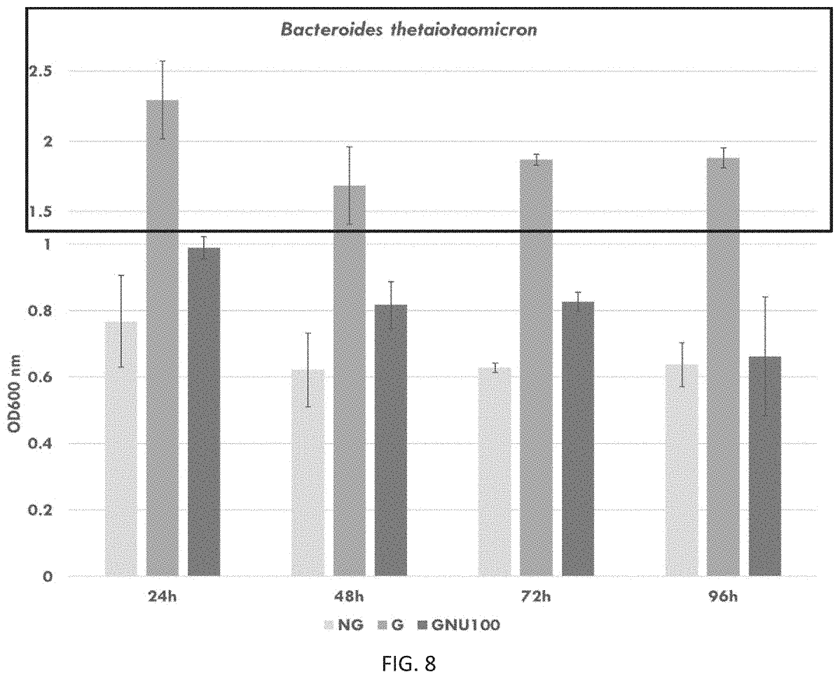

Bacterial growth in the presence of a composition of a claimed invention in liquid minimal media, GNU100 (15 mg/ml), was compared to bacteria growth in liquid minimal media (no glucose) and liquid minimal media with glucose (glucose). GNU100 in the form of a dried powder was obtained by the process of Example 1. Each sample was added to 200 μl medium and inoculated with 5 μl of Bifidobacterium bifidum (FIG. 3), Bifidobacterium animalis subsp. lactis (FIG. 4), Bifidobacterium breve (FIG. 5), Lactobacillus acidophilus (FIG. 6), Akkermansia muciniphila (FIG. 7) or Bacteroides thetaiotaomicron (FIG. 8). Each sample was prepared in triplicate. The bacterial growth was determined by measuring the optical densities (OD) at 600 nm in a spectrophotometer after 24 h, 48 h, 72 h and 96 h of growth starting with an OD of 0.05.

FIG. 3 illustrates that supplementing minimal media with GNU100 results in growth of Bifidobacterium bifidum, as measured by OD, superior to growth of Bifidobacterium bifidum in no glucose and glucose at 72 hours. Further, FIG. 3 illustrates that GNU100 supplementation results in similar growth of Bifidobacterium bifidum to no glucose at all other time points. It is believed that glucose is not an ideal energy source for gut microbiota, as glucose tends to inhibit the growth of certain beneficial bacteria in the microbiota, such as Akkermansia muciniphila.

FIG. 4 illustrates that supplementing minimal media with GNU100 results in growth of Bifidobacterium animalis, as measured by OD, superior to growth of Bifidobacterium animalis in no glucose at 96 hours. Further, FIG. 3 illustrates that GNU100 supplementation results in similar growth of Bifidobacterium animalis to no glucose at all other time points.

FIG. 5 illustrates that supplementing minimal media with GNU100 results in growth of Bifidobacterium breve, as measured by OD, similar to growth of Bifidobacterium breve in no glucose at all time points.

FIG. 6 illustrates that supplementing minimal media with GNU100 results in growth of Lactobacillus acidophilus, as measured by OD, superior to growth of Lactobacillus acidophilus in no glucose at 48 and 96 hours.

FIG. 8 illustrates that supplementing minimal media with GNU100 results in growth of Bacteroides thetaiotaomicron, as measured by OD, superior to growth of Bacteroides thetaiotaomicron in medium without glucose at all time points.

The results shown in FIGS. 3-6 and 8 show that compositions of the claimed invention sustain higher growth rates for some beneficial bacteria at different time points that minimal media or minimal media containing glucose. Thus, these results suggest that beneficial bacteria are capable of utilizing glycans attached to peptides or proteins, especially after other energy sources are exhausted.

### Example 5 Akkermansia muciniphilia Growth in GNU100 Supplemented Media

Growth of Akkermansia muciniphila with a composition of a claimed invention in liquid minimal media, GNU100, was compared to Akkermansia muciniphila in liquid minimal media (NG) and liquid minimal media with glucose (G). GNU100 was obtained by the process of Example 1. Each sample was inoculated with 5 μl of Akkermansia muciniphila to 200 μl of medium.

FIG. 7 shows that GNU100 supplementation of minimal media results in growth of Akkermansia muciniphila. Akkermansia muciniphila did not grow in liquid minimal media (no glucose) and liquid minimal media with glucose (glucose).

The results of Examples 4 and 5, taken together, show that compositions of the claimed invention are suitable energy sources for extended growth of numerous beneficial bacteria, and are an especially superior energy source for extended growth of Akkermansia muciniphila. Further, the inventors have found that compositions of the claimed invention do not promote growth of Escherichia coli or Salmonella strains, further showing that the compositions of the claimed invention are superior additives for food stuffs and pet foods.

### Example 6 Dog Food Supplemented with GNU100

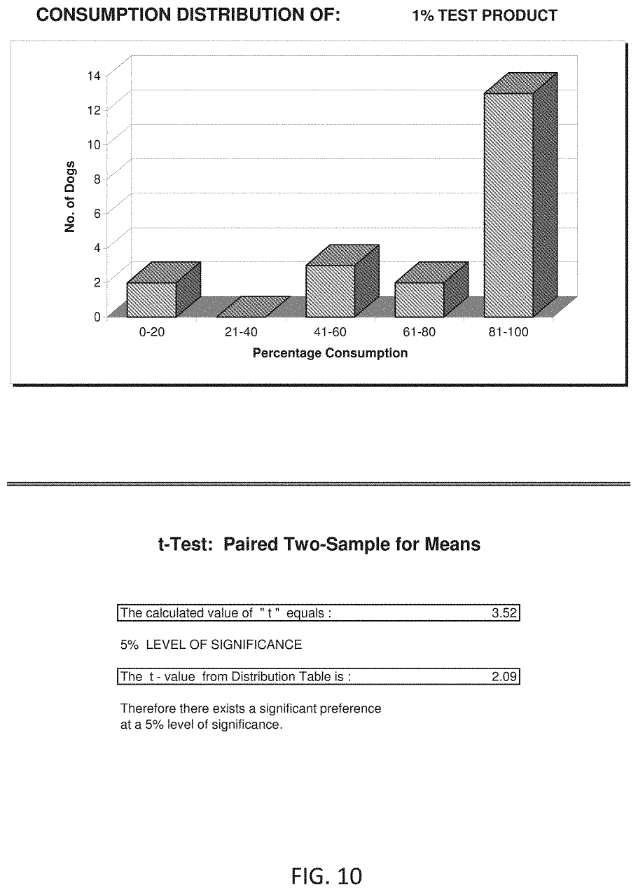

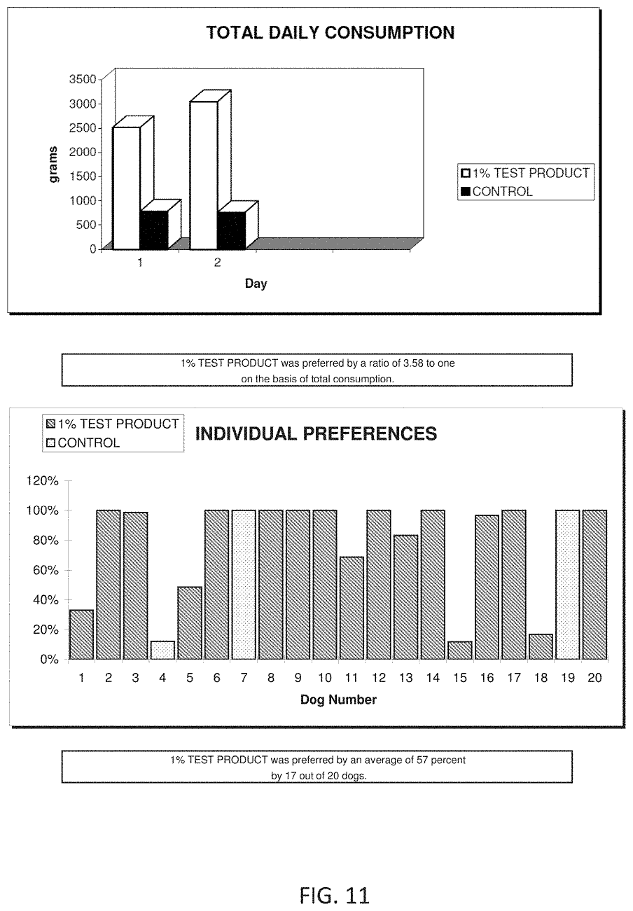

Twenty healthy English Pointers or Beagles were weighed and randomly assigned to separate kennels. 400 grams of dog food supplemented with 5% fat (control) was offered in one bowl and 400 grams of dog food supplemented with 5% fat and 1% GNU100 (1% Test Product) was simultaneously offered in a second bowl. After approximately 20 minutes, the bowls were removed and the weight of the remaining food measured. The following day, the test was repeated with the left and right positions of the control and 1% Test Product bowls reversed, to account for left-right bias by the dogs. The results are shown in FIGS. 10-11 and the following Table 5:

TABLE 5
CONSUMPTION IN GRAMS

| DOG | WT. | 1% TEST PRODUCT | | CONTROL | |
|---|---|---|---|---|---|
| # | Kg. | DAY 1 | DAY 2 | DAY 1 | DAY 2 |
| 1 | 11.6 | 0 | 171 | 86 | 0 |
| 2 | 20.6 | 153 | 399 | 0 | 0 |
| 3 | 10.6 | 123 | 155 | 2 | 0 |
| 4 | 11.6 | 171 | 0 | 0 | 219 |
| 5 | 15.3 | 170 | 200 | 45 | 83 |
| 6 | 10.8 | 107 | 112 | 0 | 0 |
| 7 | 14.1 | 0 | 0 | 145 | 183 |
| 8 | 9.8 | 100 | 150 | 0 | 0 |
| 9 | 18.1 | 176 | 238 | 0 | 0 |
| 10 | 14.6 | 0 | 165 | 0 | 0 |
| 11 | 17.4 | 255 | 201 | 53 | 32 |
| 12 | 10.3 | 167 | 149 | 0 | 0 |
| 13 | 8.4 | 13 | 64 | 7 | 0 |
| 14 | 12.1 | 218 | 156 | 0 | 0 |
| 15 | 16.0 | 0 | 230 | 181 | 0 |
| 16 | 17.1 | 294 | 275 | 6 | 4 |
| 17 | 15.7 | 127 | 130 | 0 | 0 |
| 18 | 18.1 | 186 | 0 | 88 | 44 |
| 19 | 24.1 | 0 | 0 | 178 | 200 |
| 20 | 21.3 | 261 | 258 | 0 | 0 |
| TOTAL | 297.6 | | | | |

| | | | | |
|---|---|---|---|---|
| TOTALS . . . | 2521 | 3053 | 791 | 765 |

| | | |
|---|---|---|
| GRAND TOTAL . . . | 5574 = 9.4 g/Kg/Day | 1556 = 2.6 g/Kg/Day |

1% TEST PRODUCT was approached first on 21 out of 40 occasions.
1% TEST PRODUCT was consumed first on 31 out of 40 occassions.

As shown in FIG. 10, thirteen out of twenty dogs ate 81% or more of the dog food supplemented with 1% GNU100. Furthermore, the dogs consumed dog food supplemented with 1% GNU100 at a ratio of 3.58 to 1 as compared to non-supplemented dog food (FIG. 11, top panel). Finally, dog food supplemented with 1% GNU100 was preferred by an average of 57% compared to non-supplemented dog food by 17 out of the 20 dogs tested (FIG. 11, bottom panel).

### Example 6 Cat Food Supplemented with GNU100

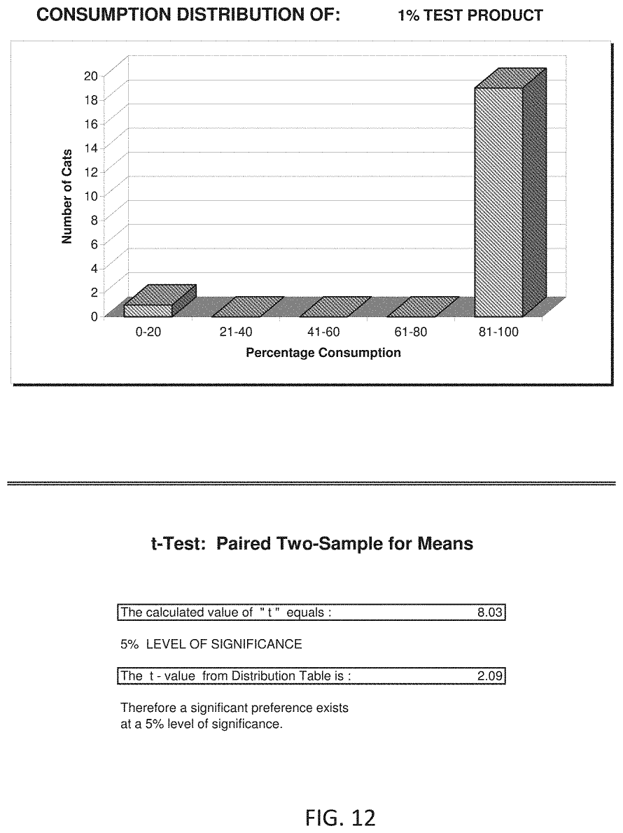

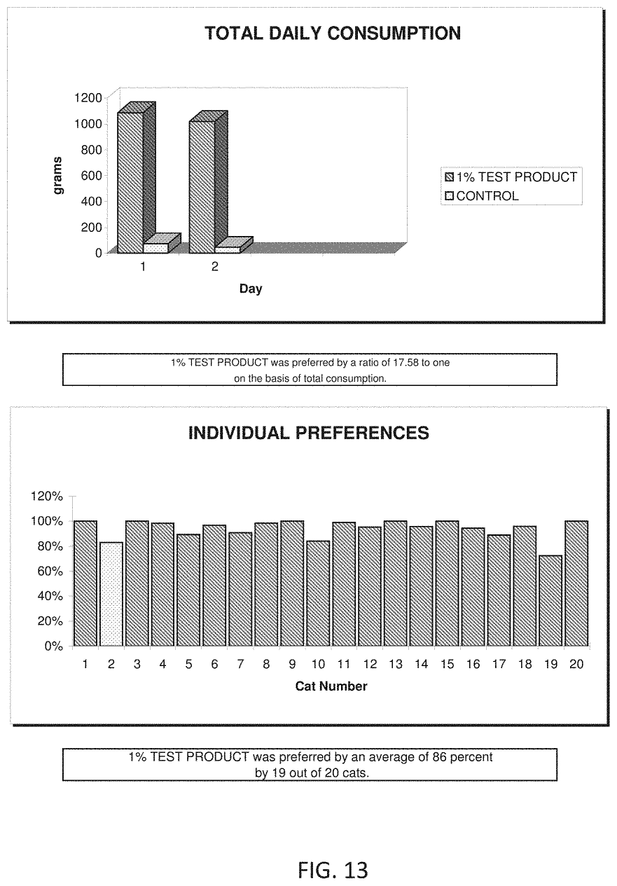

Twenty healthy cats were weighed and randomly assigned to separate kennels. 110 grams of cat food (control) was offered in one bowl and 110 grams of cat food supplemented with 1% GNU100 (1% Test Product) was simultaneously offered in a second bowl. After feeding, the bowls were removed and the weight of the remaining food measured. The following day, the test was repeated with the left and right positions of the control and 1% Test Product bowls reversed, to account for left-right bias by the cats. The results are shown in FIGS. 12-13 and the following Table 6:

TABLE 6
CONSUMPTION IN GRAMS

| CAT | WT. | 1% TEST PRODUCT | | CONTROL | |
|---|---|---|---|---|---|
| # | Kg. | DAY 1 | DAY 2 | DAY 1 | DAY 2 |
| 1 | 4.0 | 64 | 71 | 0 | 0 |
| 2 | 4.6 | 4 | 1 | 37 | 17 |
| 3 | 3.2 | 56 | 1 | 0 | 0 |
| 4 | 3.3 | 55 | 59 | 0 | 1 |
| 5 | 5.3 | 60 | 46 | 0 | 6 |
| 6 | 2.9 | 22 | 36 | 1 | 0 |
| 7 | 4.9 | 50 | 52 | 5 | 0 |
| 8 | 6.0 | 72 | 59 | 1 | 0 |
| 9 | 4.8 | 69 | 53 | 0 | 0 |
| 10 | 4.7 | 105 | 104 | 18 | 0 |
| 11 | 4.3 | 98 | 98 | 1 | 0 |
| 12 | 5.1 | 59 | 59 | 0 | 3 |
| 13 | 3.5 | 25 | 18 | 0 | 0 |
| 14 | 4.2 | 47 | 42 | 1 | 1 |
| 15 | 4.6 | 54 | 39 | 0 | 0 |
| 16 | 6.0 | 59 | 42 | 0 | 3 |
| 17 | 4.4 | 31 | 37 | 1 | 3 |
| 18 | 6.0 | 87 | 97 | 4 | 0 |
| 19 | 5.4 | 48 | 58 | 5 | 12 |
| 20 | 2.8 | 23 | 49 | 0 | 0 |
| TOTAL | 90.0 | | | | |

| | | | | |
|---|---|---|---|---|
| TOTALS . . . | 1088 | 1021 | 74 | 46 |

| | | |
|---|---|---|
| GRAND TOTAL . . . | 2109 = 11.7 g/Kg/Day | 120 = 0.7 g/Kg/Day |

1% TEST PRODUCT was approached first on 28 out of 40 occasions.
1% TEST PRODUCT was consumed first on 38 out of 40 occassions.

As shown in FIG. 12, nineteen out of twenty cats ate 81% or more of the cat food supplemented with 1% GNU100. Furthermore, the cats consumed cat food supplemented with 1% GNU100 at a ratio of 17.58 to 1 as compared to non-supplemented cat food (FIG. 13, top panel). Finally, cat food supplemented with 1% GNU100 was preferred by an average of 86% compared to non-supplemented cat food by 19 out of the 20 cats tested (FIG. 13, bottom panel).

### Example 7—Alternate GNU100 Production (Prep #4)

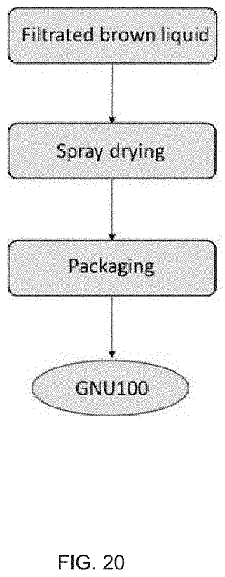

Partially purified hydrolyzed porcine gastrointestinal tract mucins were obtained from commercial sources. GNU100 was obtained by the process shown in FIG. 20 by filtration with a filter having a pore size 4-7 followed by spray drying. The resultant GNU100 composition had the following properties:
Oligosaccharides Diversity—28 Different Structures
Solubility—Water soluble (80 to 120 g/L at 25° C.)
Chemical/Physical proprieties:
   Glycopeptides and peptides 44-57%
   Ash 13-16%
   Free amino acids 30-40%
   Moisture 2-5%
   pH 5.50-6.50 (2% w/v, in DI water at 20° C.)
Microbiological:
Salmonella Negative in 25 g
Escherichia coli<10 CFU/g

The resultant GNU100 also had the following chemical properties:
B (ppm)<1
Cl tot. (ppm) 2'000
F (ppm)<500
P (ppm) 11'000
S tot. (ppm) 2'000
As (ppm)<1
Cd (ppm)<1
Pb (ppm)<1
Hg (ppm)<1

### Example 8—Investigation of GNU100 Using Short Term Single Stage Colonic Simulation

Materials and Methods

The short-term screening assay consisted of a colonic incubation of 2 different doses of GNU100 under conditions representative for the proximal colon region of a cat and a dog, with a representative bacterial inoculum. Mucin beads were also added to the reactors to simulate the mucosal environment of the colon. At the start of the short-term colonic incubation, the test ingredient, preceded by dialysis to remove amino acid fractions, was added in a concentration of 5 g/L and 10 g/L to sugar-depleted nutritional medium containing basal nutrients present in the colon. A blank, containing only the sugar-depleted nutritional medium (without fiber) was included also, to assess the background activity of the bacterial community.

As a source of the colonic microbiota, a freshly prepared fecal inoculum of a single donor was added (healthy adult dog and healthy adult cat). Incubations were performed for 48 h at 39° C., under shaking (90 rpm) and anaerobic conditions. The incubations were performed in fully independent reactors with sufficiently high volume to not only allow for a robust microbial fermentation, but also to allow collection of multiple samples over time. Sample collection enables assessment of metabolite production and enables understanding of the complex microbial interactions that are taking place. Each condition was performed in triplicate to account for biological variation, resulting in 9 independent incubations (1 blank+2 treatments) for each donor.

Canine Donor
Healthy dog, male
Breed: Boxer
Age: 4 years, 7 months
Body condition score (BCS): 4 (healthy weight)

Feline Donor
Healthy cat, male
Breed: European shorthair
Age: 14 years
Body condition score (BCS): 4 (healthy weight)

Experimental Setup

24 h dialysis of GNU100 using 0.5 kDa membranes to remove amino acid fractions (as would occur in vivo). Standard short-term colonic simulations in reactors at 39° C. including 2 treatments and a control for 2 donors (dog and cat), tested in triplicate. 2 different inoculum, i.e. 1 dog (˜SCIME) and 1 cat (˜SFIME). 3 conditions—i.e. control versus dialyzed GNU100 (5 g/L and 10 g/L). Inclusion of mucus beads to simulate mucosal environment.

Endpoints

Overall Fermentative Activity

pH: the degree of acidification during the experiment is a measure for the intensity of bacterial metabolism of the potential prebiotic (fermentation). The pH of the incubations was determined 0, 6, 24 and 48 h after starting the incubation, thus giving a rough indication on the speed of fermentation of the different test products.

Gas production: the colon incubations were performed in closed incubation systems. This allowed evaluation of the accumulation of gasses in the headspace, which can be measured with a pressure meter. Gas production is a measure of microbial activity, and thus of the speed of fermentation of the potentially prebiotic substrates. H₂ and CO₂ are the first gasses to be produced upon microbial fermentation; they can subsequently be utilized as substrates for CH₄ production, reducing the gas volume. H₂ can also be utilized to reduce sulfate to H₂S, resulting from proteolytic fermentation. As a result, N₂, O₂, CO₂, H₂ and CH₄ constitute for 99% the volume of intestinal gas. The remaining 1% consists of NH₃, H₂S, volatile amino acids and short chain fatty acids. Total gas production during incubation was determined 0, 6, 24 and 48 h after starting the incubation.

Changes in Microbial Metabolites

The following analyses enabled assessment of the kinetics in the production of bacterial metabolites upon fermentation of the prebiotic compound.

Short chain fatty acid analysis (0, 6, 24 and 48 h): SCFA production is a measure of the microbial carbohydrate metabolism (acetate, propionate and butyrate) or protein metabolism (branched SCFA) and can be compared to typical fermentation patterns for normal GI microbiota.

Lactate (0, 6, 24 and 48 h): the human intestine harbors both lactate-producing and lactate-utilizing bacteria. Lactate is produced by lactic acid bacteria and decreases the pH of the environment, thereby also acting as an antimicrobial agent. Protonated lactic acid can penetrate the microbial cell after which it dissociates and releases protons within the cell, resulting in acidification and microbial cell death. It can also be converted into propionate and butyrate by other microorganisms.

Ammonium (0, 24 and 48 h): Ammonium is a product of proteolytic degradation, which results in the production of potentially toxic or carcinogenic compounds such as p-cresol and p-phenol. It can be used as an indirect marker for low substrate availability. Since it is only produced towards the end of the incubation, it is not measured after 6 h.

Microbial Sequencing

Total DNA extracts from the colonic simulations were obtained using the CTBA method. Lumen total DNA samples were collected at 0 h, 24 h and 48 h after the beginning of the incubation (ABI). In order to gain insight into the mucosal microenvironment, total DNA from mucus beads was extracted at 48 h in addition to the lumen samples. The extracted total DNA was processed by Bioinnovation Solutions using the PETSEQ workflow for bacterial detection in cats and dogs. This consists of molecular assay for library preparation, sequencing, and data analysis and interpretation.

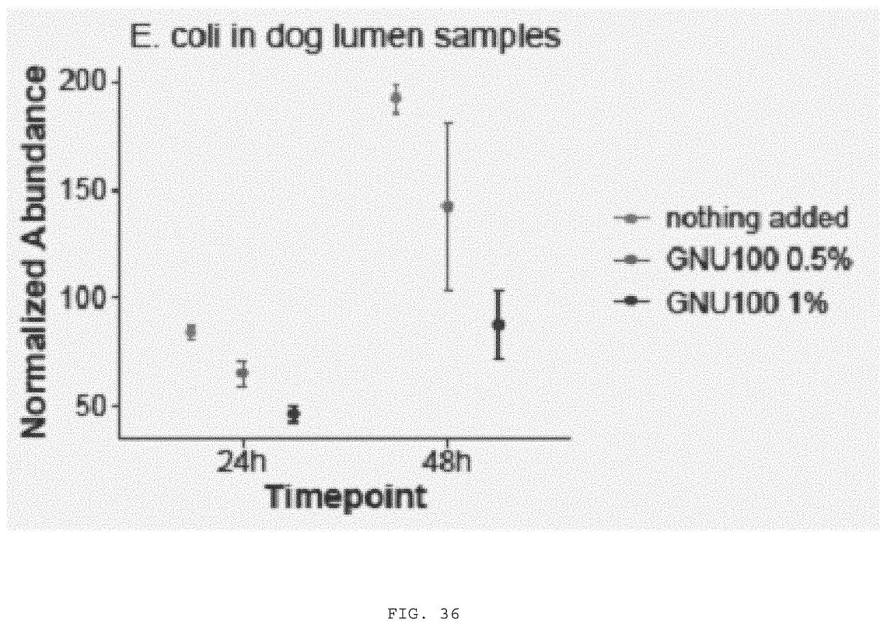

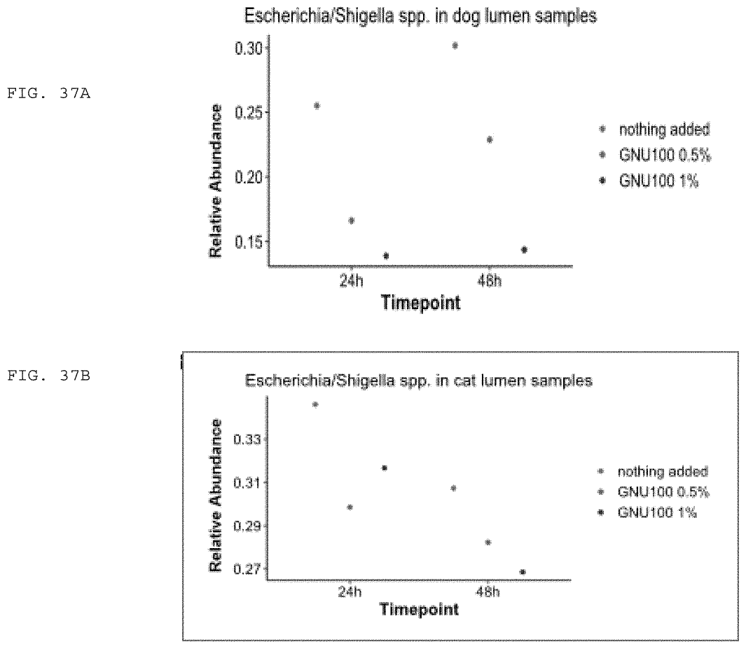

The addition of GNU100 was enough to significantly decrease the levels of Escherichia coli species found in dog lumen samples at both 24 h and 48 h ABI (FIG. 36). No changes in Escherichia coli abundance were observed in cat lumen samples upon administration of GNU100 (data not shown). PETSEQ analysis highlighted an important dose-dependent decrease of Escherichia bacteria species, in lumen samples treated with GNU100 when compared to the control sample (FIG. 37).

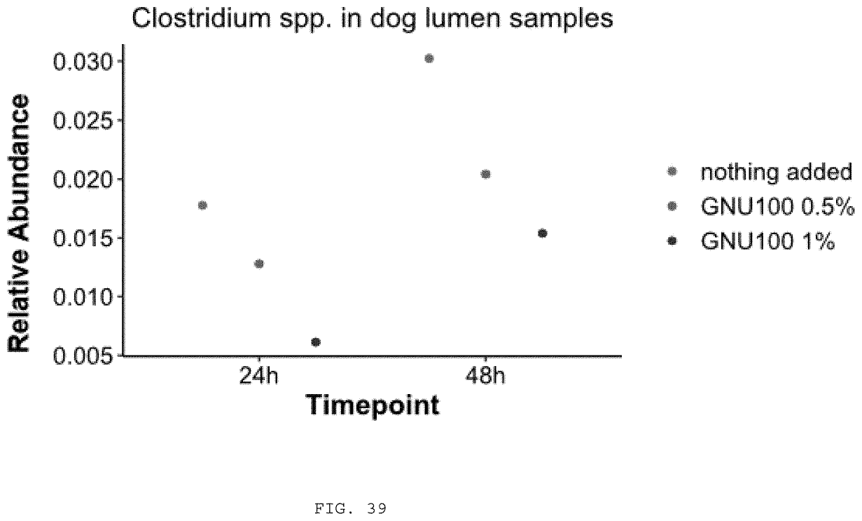

Salmonella spp. Was detected at low relative abundance in all dog and cat lumen samples. The addition of GNU100 caused a strong reduction of abundance in both animals in a dose-dependent manner. The effect was particularly visible at 48 h ABI using the highest dosage of the product. The abundance Clostridium was also reduced in dog lumen samples treated with GNU100 (FIG. 39).

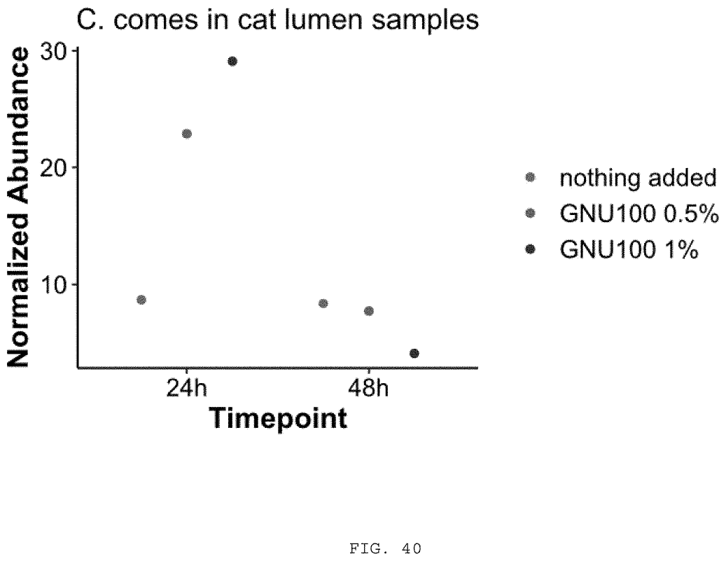

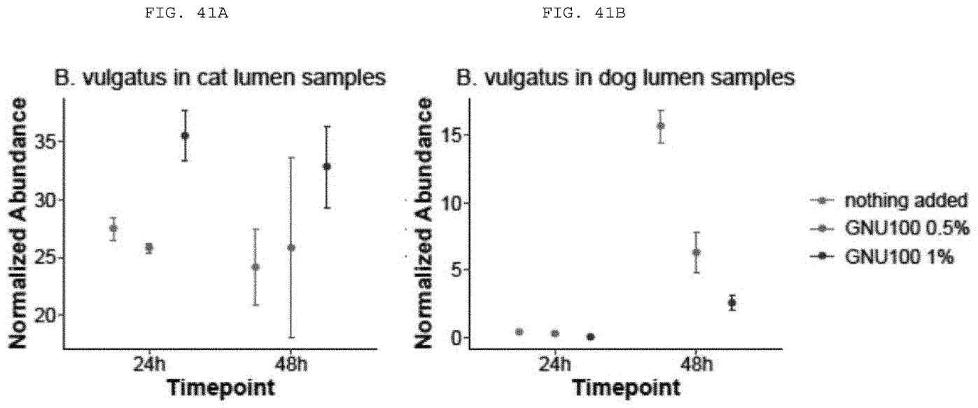

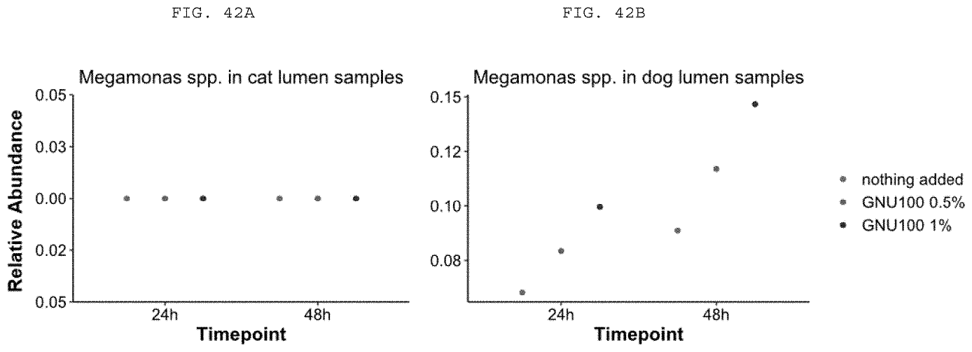

PetSeq showed an increased abundance of Bacteroides spp. in cat lumen samples (FIG. 40) treated with GNU100. Increase of Bacteroides vulgatus species (FIG. 41) is one of the Bacteroides species increased. Dog samples, on the other hand, showed a reduced abundance of Bacteroides in all samples and no correlation between Bacteroides vulgatus and propionate production. Propionate production in dog samples correlates with Megamonas increase upon GNU100 supplement (FIG. 42). In cat samples, members of the Megamonas genus were not detected. Species-level analysis did not highlight any bacteria belonging to the Megamonas genus that correlated with propionic acid increase.

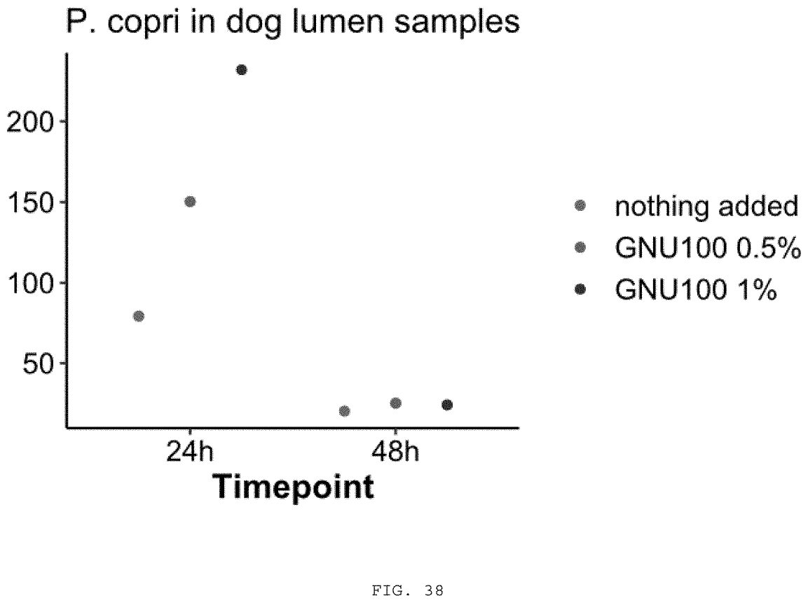

Increase in abundance of different genera that are known acetate producers coincides with the increase in acetate production in both cat and dog samples. In dog, there is an increase shown for Ruminococcus spp. and Prevotella copri (FIG. 38) with increased GNU100 dose. These bacteria produce acetate from pyruvate.

Coprococcus comes, which is a known butyrate producer, coincides with the increase in butyrate in cat samples (FIG. 40).

Sialylated glycans have been shown to play an important role in modulation of gut microbiome. GNU100 antimicrobial proprieties are conferred by its unique formula that includes 30 different glycans, 10 of which have been identified as sialylated glycans. Escherichia coli is a type of bacteria commonly found within the intestinal tract. Several Escherichia coli strains have been associated to intestinal disease, thus making it important to actively monitor the species. Indeed, the addition of GNU100 to dog intestinal lumen simulations greatly decreased the relative abundance of the species if compared to untreated control samples. Moreover, the magnitude of the reduction is directly linked to the quantity of product used, confirming the specificity of the observed effect.

Contrarily to cats, healthy dogs are not expected to carry detectable amounts of Escherichia coli pathogenic strains, thus, it comes without surprise that the reduction of Escherichia coli mpk, a non-pathogenic strain, is the major cause of Escherichia coli depletion in dog lumen samples treated with GNU100. These results suggest that GNU100 is particularly effective in limiting the growth of Escherichia coli in dog intestinal simulations.

Contrary to what was shown for dogs, Escherichia coli abundance seems not to be affected by GNU100 in cat samples. However, 16S analysis showed a clear reduction of the Escherichia/Shigella spp in both cat and dog samples, suggesting that different species belonging to the same genus, other than Escherichia coli, are effectively inhibited by the addition of GNU100.

Bacterial species belonging to the Salmonella and Clostridium genera can reside in the intestine of healthy animals with relatively low abundance, without causing any visible symptoms to the host. However, sudden changes in the gut homeostasis can promote the growth of these potential pathogens and cause serious gastric diseases. Thus, it is important to keep the level of potential pathogen populations under control. Low levels of Salmonella and Clostridium were detected in dog lumen samples. Interestingly, all GNU100 treated samples showed a decrease of both genera suggesting that GNU100 might be able to actively reduce potential pathogenic species. The relative abundance of Salmonella was decreased in cat lumen samples, although the effect of GNU100 treatment was less visible by the overall low abundance of Salmonella spp. in cat samples.

Overall, these results suggest that a supplement of GNU100 could help reduce the burden of potentially dangerous bacteria in the cat/dog intestinal tract.

Short chain fatty acid (SCFA) production results from microbial carbohydrate metabolism in the colon and is related with various health effects. The most abundantly produced SCFAs include acetate, propionate and butyrate. Whereas acetate can be used as an energy source by the host and as a potential substrate for lipid synthesis in the body, propionate reduces cholesterol and fatty acid synthesis in the liver (beneficial effect on metabolic homeostasis). Butyrate on the other hand, is a major energy source for colonocytes.

The in vivo simulation performed show a dose-dependent increase of acetate, propionate and butyrate with GNU100 and the stimulation of propionate and butyrate production suggests that GNU100 was metabolized by the bacteria and that cross-feeding mechanisms were triggered by the treatment.

Various bacterial species and/or genera are known to produce these SCFAs and therefore have a positive impact on the gut health. Herein, several species that are known SCFA producers are identified having a dose-dependent increase with GNU100. This correlates with the increase of acetate, propionate and butyrate shown herein. For dog lumen samples, those bacteria were Megamonas spp.; for cat the identified bacteria were Bacteroides spp. (including Bacteroides vulgatus) and Coprococcus comes. These bacteria seem to be able to utilize GNU100, produce SCFA and ultimately leading to a healthier digestive system.

Microbial Metabolic Activity

Overall Fermentative Activity

pH Decrease

Monitoring the pH during a colonic incubation provides a good indication of the production of SCFA, lactate and ammonium (NH₄+). In general, a pH drop is observed during the first 24 h of incubation due to the formation of SCFA/lactate. This pH drop is often followed by a pH increase during the last 24 h of incubation due to proteolytic fermentation, which results in the production of amongst others NH₄+, and due to conversion of stronger acids into weaker acids through cross-feeding (for instance acetate/lactate-topropionate/butyrate conversion).

The following observations were made:

Overall the strongest pH decrease was observed during the first 6 h of incubation for both donors. The pH decrease was similar between blanks and treatments (regardless of product concentrations). The extent of the pH decrease was more pronounced for the cat than the dog.

During the 6-24 h timeframe a pH increase was seen for both donors. The increase was similar between blanks and treatments. In this case also, the extent of the pH increase was most pronounced for the cat.

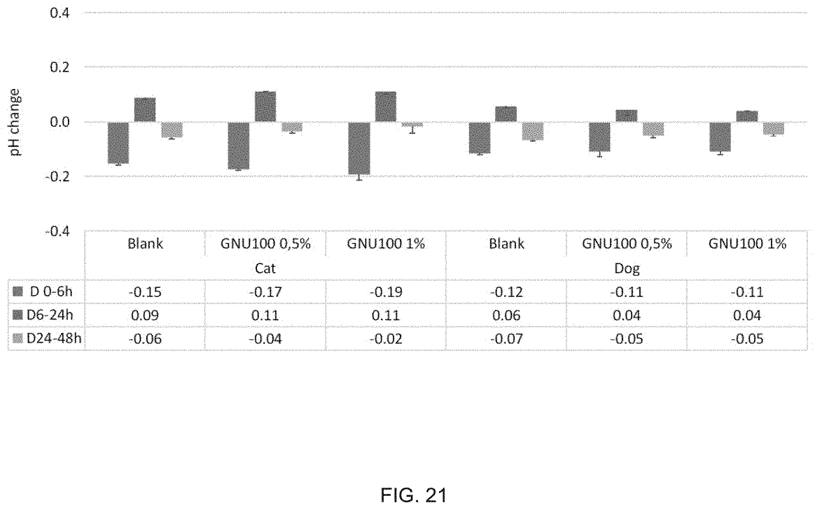

During the last 24 h of incubation a mild pH decrease was observed for both donors. The pH decrease was similar between blanks and treatments. See FIG. 21.

Gas Production

Besides pH decrease, gas production is a measure of overall microbial activity, and thus of speed of fermentation. The blank yielded the lowest gas pressures. Any gas produced in the blank was likely due to proteolytic fermentation of peptides and proteins in the background medium.

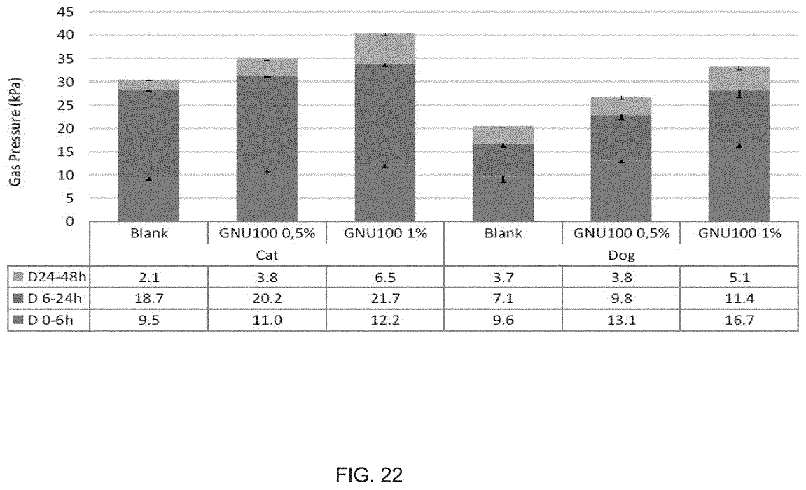

Both product concentrations stimulated gas production compared to the blank incubation in both donors, indicative of product fermentation. A dose-response relation was observed for both donors. Gas production was most pronounced in the cat during the 6-24 h timeframe, and between 0-6 h in the dog. See FIG. 22.

Short-Chain Fatty Acids

SCFA production results from carbohydrate metabolism in the colon and is related with various health effects. The most abundantly produced SCFAs include acetate, propionate and butyrate. Whereas acetate can be used as an energy source for the host and as a potential substrate for lipid synthesis in the body, propionate reduces cholesterol and fatty acid synthesis in the liver (beneficial effect on metabolic homeostasis). Butyrate on the other hand, is a major energy source for colonocytes and induces differentiation in these cells (related to cancer prevention). Positive effects of the investigated substrates on SCFA production therefore include an increase of acetate, propionate and/or butyrate.

Acetate

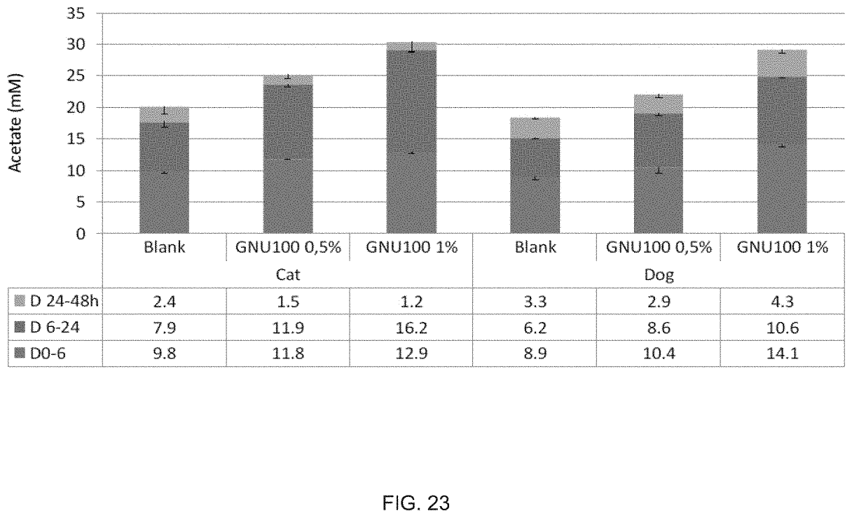

Acetate can be produced by many different gut microbes (e.g. Bifidobacterium, Bacteroides, . . . ). GNU100 stimulated acetate production, as illustrated by the higher acetate levels in the treatment incubations than the blank incubations in cat and dog. A dose-response relation was observed for both donors, thus consistently yielding higher acetate concentrations for the 1% dose. Acetate production mostly occurred during the first 24 h of the incubation. See FIG. 23.

Propionate

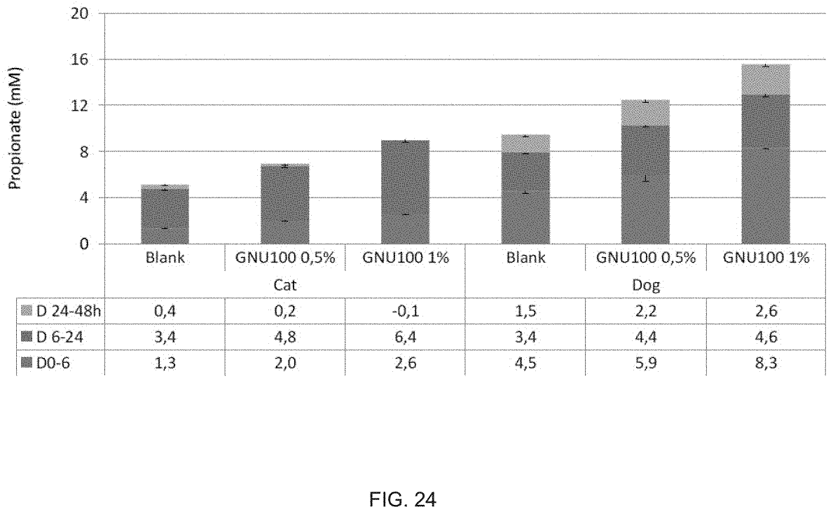

Propionate can be produced by a wide range of gut microbes, with the most abundant propionate producers being Bacteroides spp. (phylum=Bacteroidetes), Veillonellaceae (phylum=Firmicutes) and Akkermansia muciniphila (phylum=Verrucomicrobia). GNU100 stimulated propionate production, as illustrated by the higher propionate levels in the treatment incubations than the blank incubations in cat and dog. A dose-response relation was observed for both donors, thus consistently yielding higher concentrations for the 1% dose. Propionate production mostly occurred during the first 24 h of the incubation. See FIG. 24.

Butyrate

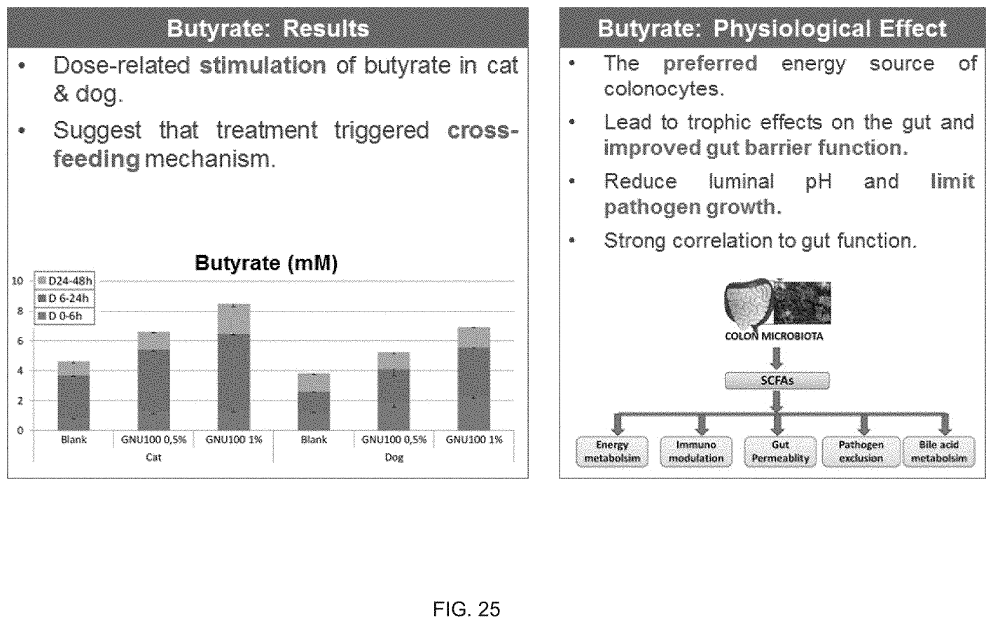

Butyrate is produced by members of the Clostridium clusters IV and XIVa (phylum=Firmicutes). In a process called cross-feeding, these microbes convert acetate and/or lactate (along with other substrates) to the health-related butyrate. GNU100 stimulated butyrate production, as illustrated by the higher butyrate levels in the treatment incubations than the blank incubations in cat and dog. A dose-response relation was observed for both donors, thus consistently yielding higher concentrations for the 1% dose. Butyrate production mostly occurred between 6-24 h of incubation. See FIG. 25.

Lactate

The human intestine harbors lactate-producing and lactate-utilizing bacteria. Lactate is produced by lactic acid bacteria (bifidobacteria and lactobacilli) and decreases the pH of the environment. Especially at low pH values, lactate can exert strong antimicrobial effects against pathogens, as protonated lactic acid can penetrate the microbial cell, after which it dissociates and releases protons within the cell, resulting in acidification and microbial cell death. Another beneficial effect of lactate results from its conversion to butyrate and/or propionate by specific micro-organisms. As different microbial species thus produce and convert lactate, an increase of lactate concentration can both result from an increased production as well as a decreased conversion. Therefore, one needs to be cautious with interpretation of lactate data.

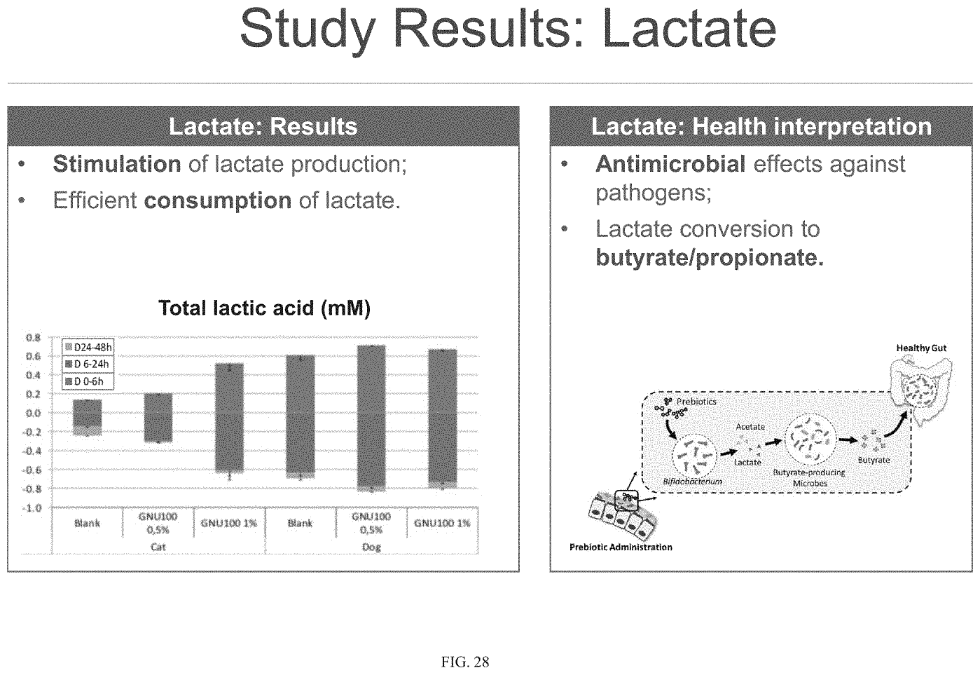

Lactate production was generally low. During the first 6 h of incubation, the lactate production rate exceeded the lactate consumption rate, leading to accumulation of lactate. Lactate production was moderately stimulated by the treatment with GNU100 in dog and cat. A dose-response relation was less pronounced than observed for SCFA production. Any lactate produced during the first 6 h was efficiently consumed by the end of the incubation for both donors. This is indicative of efficient lactate conversion. See FIG. 28.

Markers for Protein Metabolism: Ammonium and Branched SCFA

Less abundant SCFA include branched SCFA (isobutyrate, isovalerate and isocaproate). Ammonium and branched SCFA production results from proteolytic microbial activity, which is associated with formation of toxic by-products such as p-cresol. Therefore, high branched SCFA and ammonium production in the colon has been associated with detrimental health effects. As a consequence, products that reduce branched SCFA and ammonium production are considered health-beneficial.

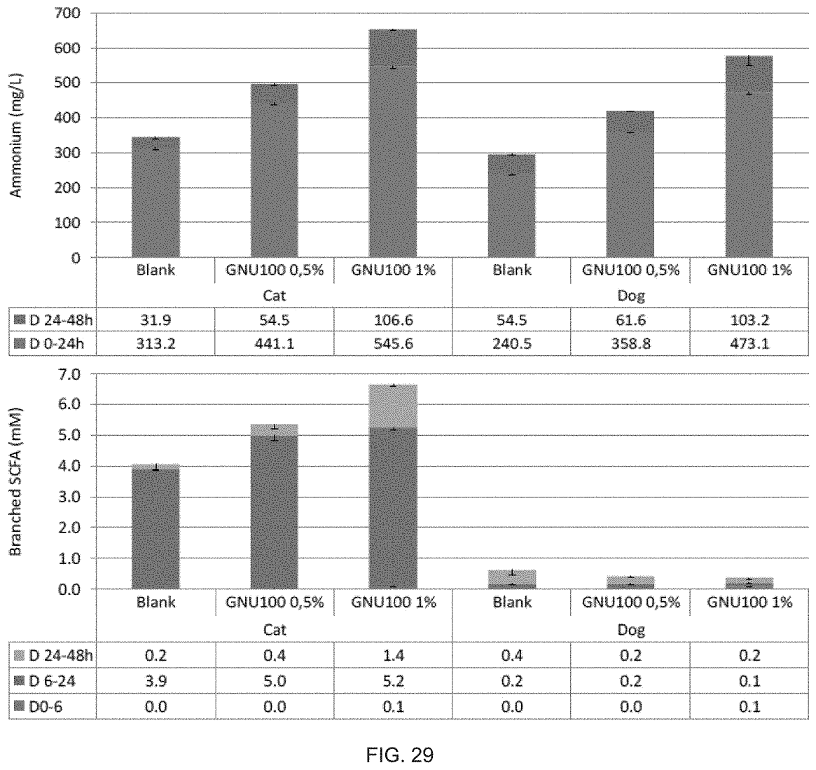

GNU100 is known to contain glycopeptides; fermentation by the gut microbiota was thus expected to result in elevated ammonium levels. Indeed, fermentation of GNU100 was associated with an increase in ammonium concentrations in cat and dog, mostly during the first 24 h of incubation (which was the timeframe during which product fermentation mainly took place). A dose-response relation was observed. Increased ammonium concentrations explain the mild pH decreases observed, as produced ammonium neutralized medium acidification induced by SCFA production. See FIG. 29, top panel.

Branched SCFA production was virtually absent in the incubations with the dog. However, GNU100 stimulated branched SCFA production in the feline incubations, with a higher dose resulting in a higher metabolite concentration. See FIG. 29, bottom panel.

### Example 9—Preparation and Analysis of GNU100 (Prep #5)

Partially purified hydrolyzed porcine gastrointestinal tract mucins were obtained from commercial sources and stabilized at a pH of 5.5 using sulfuric acid or sodium hydroxide, as appropriate. The stabilized mucins were then centrifuged at low speed (500 to 10,000×g) to remove large insoluble particles, fats, and lipids. The mucins were then desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further purified by partially filtering through a 0.45 (1M Polyethersulfone (PES) filter (Millipore Sigma) until the flow rate was reduced in order to remove some amino acids and salts, and the retentate collected.

One hundred ml of the retentate was filtrated on Whatman filter paper (diameter 110 mm, pore size 4-7 μm) by suction using a Büchner funnel. About 100 ml of filtrate (brown liquid) was obtained. The solid residue was discarded, and the filtrate dried under rotary evaporator at 50 mbar and 50° C. m=31.8 g. Total yield=31.8%. Dry substance yield=88% to yield a powder composition of the claimed invention labeled GNU100. The powder composition was white to yellow with an amino acid smell and had a 2-5% moisture content. The water solubility of the powder was greater than 120 g/L at 25° C.

Analysis of glycan content of GNU100-O-glycans were released from glycopeptides in GNU100 by β-elimination in 50 mM NaOH and 0.5M NaBH4. If needed, pH was adjusted to above 12, the required pH for a successful release reaction. The samples were incubated in 50° C., with the lids loosely tightened. On day 2, the samples were slowly neutralized with concentrated acetic acid (HAc). Aliquots (20 ul) of the samples were desalted using cation exchange resin (AG50W×8) packed onto a ZipTip C18 tip. After drying the samples in SpeedVac, 50 ul 1% Acetic Acid (HAc) in methanol was added five times to remove residual borate by evaporation.

Released glycans were resuspended in water and analyzed by liquid chromatograph-electrospray ionization tandem mass spectrometry (LC-ESI/MS). The oligosaccharides were separated on a column (10 cm×250 inn) packed in-house with 3 inn porous graphite particles (Hypercarb, Thermo-Hypersil, Runcorn, UK). The oligosaccharides were injected on to the column and eluted with an acetonitrile gradient (Buffer A, 10 mM ammonium bicarbonate; Buffer B, 10 mM ammonium bicarbonate in 80% acetonitrile); Buffer C: 0.1% HAc. The gradient (0-45% Buffer B) was eluted for 30 min, followed by 8 min with 100% Buffer B, followed by 10 min with 0.1% HAc, and equilibrated with Buffer A in the next 15 min. A 40 cm×50 inn i.d. fused silica capillary was used as transfer line to the ion source.

The samples were analyzed in negative ion mode on a LTQ linear ion trap mass spectrometer (Thermo Electron, San José, CA), with an IonMax standard ESI source equipped with a stainless steel needle kept at −3.5 kV. Compressed air was used as nebulizer gas. The heated capillary was kept at 270° C., and the capillary voltage was −50 kV. Full scan (m/z 380-2000, two microscan, maximum 100 ms, target value of 30,000) was performed, followed by data-dependent MS² scans (two microscans, maximum 100 ms, target value of 10,000) with normalized collision energy of 35%, isolation window of 2.5 units, activation q=0.25 and activation time 30 ms. The threshold for MS² was set to 300 counts. Data acquisition and processing were conducted with Xcalibur software (Version 2.0.7).

TABLE 7
Oligosaccharides structures in GNU100 as obtained via LCS MS.

| Name ^{[1]} | Composition ^{[2]} | Putative structures ^{[3]} | RT ^{[4]} |
|---|---|---|---|
| 384 | Hex1HexNAc1 | Galβ1-3GalNAcol | 7.8 |
| 425 | HexNAc2 | GlcNAcβ1-6GalNAcol | 8.1, 9.9 |
| 513 | NeuAc1HexNAc1 | NeuAcα2-6HalNAcol | 10.5 |
| 529 | NeuGc1HexNAc1 | NeuGcα2-6GalNAcol | 10.3 |
| 530 | Hex1HexNAc1deHex1 | Fucα1-2Galβ1-33GalNacol | 18.5 |
| 587-1 | Hex1HexNAc2 | Gal + GlcNAcβ1-6GalNAcol | 10.1 |
| 587-2 | Hex1HexNAc2 | Galβ1-3(GlcNAcβ1-6)GalNAcol | 10.4 |
| 587-3 | Hex1HexNAc2 | Galβ1-3GlcNAcβ1-6GalNAcol | 11.2 |
| 587-4 | Hex1HexNAc2 | Galβ1-3(GlcNAcβ1-6)GalNAcol | 12.01 |
| 667 | Hex1hexNAc2Sul1 | Galβ1-3(6SGlcNAβ1-6)GalNAcol | 12.3 |
| 675-1 | NeuAc1Hex1HexNAc1 | Galβ1-3(NeuAcα2-6)GalNAcol | 11.1 |
| 675-2 | NeuAc1Hex1HexNAc1 | NeuAcαα2-3Galβ1-3GalNAcol | 13.2 |
| 691-1 | NeuGc1Hex1HexNAc1 | Galβ1-3(NeuAcα2-6)GalNAcol | 11 |
| 691-2 | NeuGc1Hex1HexNAc1 | NeuGcα2-3Galβ1-3GalNAcol | 12.8 |
| 716-1 | NeuAc1HexNAc2 | HexNAc-(NeuAcα2-6)GalNAcol | 11.3 |
| 716-2 | NeuAc1HexNAc2 | HexNAc-(NeuAcα2-6)GalNAcol | 13.8 |
| 732 | NeuGc1HexNAc2 | HexNAc-(NeuGcα2-6)GalNAcol | 11.1 |
| 733-1 | Hex1HexNAc2deHex1 | Fucα1-2(GalNAcα1-3)Galβ1-3GalNAcol | 13.21 |
| 733-2 | Hex1HexNAc2deHex1 | Fucα1-2Galβ1-4GlcNAcβ1-6GalNAcol | 15.4 |
| 733-3 | Hex1HexNAc2deHex1 | Fucα1-2Galβ1-3(GlcNAcβ1-6)GalNAcol | 18.9 |
| 813 | Hex1HexNAc2deHex1Sul1 | Fucα1-2Galβ1-3(6S-GlcNAcβ1-6)GalNAcol | 21.7 |
| 821 | NeuAc1Hex1HexNAc1deHex1 | Fucα1-2Galβ1-3(NeuAcβ2-6)GalNAcol | 21.8 |
| 870-1 | Hex1HexNAc3Sul1 | GlcNAcβ1-3[Galβ1-4(6S)GlcNAcβ1-6]GalNAcol | 14.6 |
| 870-2 | Hex1HexNAc3Sul1 | Galβ1-4GlcNAcβ1-3[(6S)GlcNAcβ1-6]GalNAcol | 15.1 |
| 895-1 | Hex2HexNAc2deHex1 | Galβ1-3(Fucα1-2Galβ1-4GlcNAcβ1-6)GalNAcol | 15.4 |
| 1016-1 | Hex1HexNAc3deHex1Sul1 | Fucα1-2Galβ1-4(6S)GlcNAcβ1-6[GlcNAcβ1-3]GalNAcol | 14.5 |
| 1016-2 | Hex1HexNAc3deHex1Sul1 | GlcNAcβ1-3[Fucα1-2Galβ1-3(6S-)GlcNAcβ1-6]GalNAcol | 18.19 |
| 1121 | Hex2HexNAc2deHex2Sul1 | Fucα1-2Galβ1-3[Fucα1-2Galβ1-4(6S)GlcNAcβ1-6]GalNAcol | 21.8 |

Footnote:
^{[1]} The names of structures;
^{[2]} Hex, hexose; HexNAc, N-acetylhexosamine; deHex, fucose; NeuAC, N-acetylneuraminic acid; NeuGc, N-acetyl glycolylneuraminic acid; S, sulphate
^{[3]} structures are given in the text according following rules: the structure is described clockwise and left-to-right where reducing end locates righmost side (as shown in cartoon figure); “ + ” is used for uncertain location Gal, galactose; Galol, alditol form of Gal; Gal NAc, N-acetylgalactosamine; GalNAcol, alditol form of Gal NAc; GlcNAc, N-acetylglucosamine; Fuc, fucose; S, sulfate; NeuAc/NeuGc, N-acetylneuramnic acid/N-acetyl glycolylneraminic acid
^{[4]} Retention time (RT) of selected structure on LC;
Recitation of “Hex” in structures 716-1 and 716-2 correspond to Glc or Gal

Determination of Principle Sugars in GNU100—HPAEC-PAD (High-performance anion exchange chromatography with derivatization-free, pulsed amperometric detection) was performed on the GNU100 composition to determine the principal sugars in the oligosaccharide component. This result is shown in a chromatogram in FIG. 2.

Specifically, GNU100 was freeze dried to remove water and treated with TFA 2N at 5 g/L at 100° C. during 4 hours under agitation to obtain free monosaccharides. The sample was then neutralized (NaOH 19N), diluted with distilled water and filtered through an 0.24 μm filter. The resulting sample was brought to a concentration of 100 mg/L to 500 mg/L of monosaccharides and loaded on a CarboPac PA-1 (Dionex) 4×250 mm analytical column to perform HPAEC-PAD with the following parameters.

System: ICS 6000 (Dionex) with pump, electrochemical detector, thermal compartment and autosampler.

Temperature of column 17° C.

Rate of elution: 1 mL/min

Volume of sample: 20 μl

Detection: Electrochemical detection PAD with reference electrode mode Ag/Cl.

Data Acquisition Software: Chromeleon (Dionex).

Elution Gradient: NaOH from 0.18 mM to 200 mM; Sodium Acetate from 0 to 500 mM. A mixture of external standards of monosaccharides (Fuc, GalNH2, GlcNH2, Gal, Glc at 6 mg/L and 12 mg/L) was analyzed in parallel to identify and quantify each monosaccharide in the tested sample.

Based on the results of the HPAEC-PAD analysis, the principle composition and content of monosaccharides in GNU100 were determined, as shown in Table 8.

TABLE 8
Composition and content of monosaccharides in GNU 100.
Oligosaccharides quantitative composition in mg/L for 100 mg/L

| Sam- | GalNac- | | | | Galac- | | |
|---|---|---|---|---|---|---|---|
| ple | ol | Fucose | GalNH2 | GlcNH2 | tose | Glucose | Total |
| GNU | 0 | 0.169 | 0.696 | 0.915 | 0.282 | 0 | 2.062 |
| 100 | | | | | | | |

Free Amino Acids analysis of GNU100—GNU100 was dissolved in water to obtain 200 mg/ml solution. 25 μL of prepared solution was extracted with 275 μL of pre-cooled Acetonitrile (ACN):H₂O (5:1, v/v) solvent containing internal standards. This solvent and sample mixture was vortexed and incubated for 1 hour at −20° C., followed by 15 min centrifugation (at 13,000 rpm at 4° C.) to facilitate protein precipitation. The resulting supernatant was collected and analyzed using Hydrophilic Interaction Liquid Chromatography coupled to High Resolution Mass Spectrometry (HILIC-HRMS) in positive ionization mode on a Q Exactive™ Hybrid Quadrupole-Orbitrap interfaced with Thermo Accela 1250 UPLC pump and CTC PAL Analytics autosampler. Amino acids were separated using a BEH Amide, 1.7 μm, 100 mm×2.1 mm I.D. column (Waters, Massachusetts, US). The mobile phase was composed of A=10 mM ammonium formate and 0.1% FA in water and B=0.1% FA in ACN. The instrument was set to acquire over the m/z range 60-900 at 70′000 FWHM resolution.

Amino acids and derivatives were quantified by using a standard calibration curves and isotopic labeled internal standards (please Table 9 below). Data was processed using TraceFinder Clinical Research (version 4.1, Thermo Fischer Scientific).

TABLE 9
List of quantified amino acids with the concentration
range of a calibration curve and stable isotope
labeled standard used for each acid.

| Amino acids & | Stable isotope- | Concentration |
|---|---|---|
| derivatives | labeled standard | range (μM) |
| 2-Aminoadipate | Tyrosine (13C9, 15N) | 4-500 |
| Alanine | Alanine (13C3, 15N) | 20-2500 |
| alpha-Aminobutyrate | Tyrosine (13C9, 15N) | 4-500 |
| Asparagine | Asparagine (13C4) | 2-250 |
| Aspartate | Aspartate (13C4, 15N) | 1-124 |
| beta-Alanine/Sarcosine | Alanine (13C3, 15N) | 4-508 |
| Cirtulline | Cirtulline (Ureido-13C) | 8-1000 |
| Creatine | Alanine (13C3, 15N) | 2-255 |
| Creatinine | Phenylalanine (13C3, 15N) | 31-4000 |
| Guanidinoacetate | Threonine (13C4, 15N) | 4-500 |
| gamma-Aminobutyrate | Methionine (13C5, 15N) | 4-495 |
| Glutamine | Glutamate (13C5, 15N) | 39-5000 |
| Glutamate | Glutamate (13C5, 15N) | 10-1248 |
| Histidine | Histidine (13C6; 15N3) | 8-1025 |
| Hydroxyproline | Proline (13C5, 15N) | 2-300 |
| Isoleucine/Allo Isoleucine | Isoleucine (13C6, 15N) | 8-1000 |
| Kynurenine | Phenylalanine (13C3, 15N) | 4-500 |
| Leucine | Leucine (13C6, 15N) | 8-1000 |
| Methionine | Methionine (13C5, 15N) | 2-263 |
| Phenylalanine | Phenylalanine (13C3, 15N) | 2-259 |
| Pipecolate | Tyrosine (13C9, 15N) | 4-500 |
| Proline | Proline (13C5, 15N) | 8-1010 |
| Taurine | Taurine (1,2-13C2) | 39-5010 |
| Threonine | Threonine (13C4, 15N) | 5-579 |
| Tryptophan | Phenylalanine (13C3, 15N) | 2-260 |
| Tyrosine | Tyrosine (13C9, 15N) | 2-246 |
| Valine | Valine (13C5, 15N) | 8-1010 |

Elemental Analysis of GNU100 An elemental analysis of the GNU100 sample was also performed. Carbon, hydrogen and nitrogen content were determined with a CHN analyzer (PerkinElmer). Chlorine content was determined with a FX Amperometric Total Chlorine Analyzer (FoxCroft). Sulfur, phosphorus, boron and sodium were measured with a Thermo Fisher Scientific ICP-iCAP 7400 elemental analyzer. Finally, fluoride content was determined by mineralizing the sample via the Wurzchmitt method followed by using the TISAB IV reagent and a fluoride ion selective electrode (Thermo Fisher Scientific).

The results are shown in Table 10.

TABLE 10

| Elements | GNU100 |
|---|---|
| C (%) | 31.2 |
| H (%) | 7.6 |
| N (%) | 9.9 |
| B (ppm) | <1 |
| Cl tot. (ppm) | 2000 |
| F (ppm) | <500 |
| Na (ppm) | 44000 |
| P (ppm) | 11000 |
| S tot. (ppm) | 2000 |
| As (ppm) | <1 |
| Cd (ppm) | <1 |
| Pb (ppm) | <1 |
| Hg (ppm) | <1 |

### Example 10. Alternate Preparation of GNU100 (Prep #6)

Partially purified hydrolyzed porcine gastrointestinal tract mucins were obtained from commercial sources and stabilized at a pH of 5.5 using sulfuric acid or sodium hydroxide, as appropriate. The stabilized mucins were then centrifuged at low speed (500 to 10,000×g) to remove large insoluble particles, fats, and lipids. The mucins were desalinated using a dialysis membrane (Slide-A-Lyzer Dialysis Flask (2K MWCO) ThermoFisher Scientific) and then concentrated by evaporation with a rotary evaporator (Fisher Scientific) with heating to at least 80° C., forming a slurry. The slurry was further purified by partially filtering through a 0.45 μM Polyethersulfone (PES) filter (Millipore Sigma) until the flow rate was reduced in order to remove some amino acids and salts, and the retentate collected.

One hundred ml of the retentate was filtrated on Whatman filter paper (diameter 110 mm, pore size 4-7 μm) by suction using a Büchner funnel. 100 ml of filtrate (brown liquid) was obtained. The filtrate was then filtrated though a 0.22 μm filter to sterilize, and the filtrate collected. The filtrate was then dried under rotary evaporator at 50 mbar and 50° C., to yield a powder composition of the claimed invention having substantially the same properties as the GNU100 of Examples 1 and 9. However, the powder exhibited reduced growth of Escherichia coli in in vitro bacterial culture as compared to the powder of Examples 1 and 9 as shown in the following Table 11.

TABLE 11
Total Plate Count Bioburden

| Sample | Total Plate Count (1 g of sample) |
|---|---|
| GNU100 w/o 0.22 filtration | 1,600 |
| GNU100 w/0.22 filtration | <100 |

1.-20. (canceled)

21. A method of manufacturing a composition comprising a mixture of glycopeptides, comprising the following steps a)-d):
a) providing hydrolyzed gastrointestinal tract mucins or a partially purified fraction thereof having a pH of approximately 5.5, wherein gastrointestinal tract mucins have been hydrolysed without subjecting the mucins or the partially purified fraction thereof to conditions or reagents that release oligosaccharides from glycopeptides,
b) optionally concentrating the mucins,
c) partially removing substances in the mucins having a diameter of less than about 0.45 μm by filtration or centrifugation, and d) removing insoluble substances in the mucins having a diameter of greater than 7 μm by filtration or centrifugation.

22. The method according to claim 21, wherein the resulting composition comprising a mixture of glycopeptides has a water solubility of greater than or equal to 120 g/L at 25° C.

23. The method according to claim 21, wherein the resulting composition comprising a mixture of glycopeptides comprises glycopeptide-bound oligosaccharides having at least each of the different structures selected from the list of structures shown in a) to bb):
a) Galβ1-3GalNAc
b) GlcNAcβ1-6GalNAc
c) NeuAcα2-6GalNAc
d) NeuGcα2-6GalNAc
e) Fucα1-2Galβ1-3 GalNAc
f) Gal+GlcNAcβ1-6GalNAc
g) Galβ1-3(GlcNAcβ1-6)GalNAc
h) Galβ1-3GlcNAcβ1-6GalNAc
i) Galβ1-3(GlcNAcβ1-6)GalNAc
j) Galβ1-3(6SGlcNAcβ1-6)GalNAc
k) Galβ1-3(NeuAcα2-6)GalNAc
l) NeuAcαα2-3Galβ1-3GalNAc
m) Galβ1-3(NeuGcα2-6)GalNAc
n) NeuGcα2-3Galβ1-3GalNAc
o) GlcNAc-(NeuAcα2-6)GalNAc
p) GalNAc-(NeuAcα2-6)GalNAc
q) HexNAc-(NeuGcα2-6)GalNAc
r) Fucα1-2(GalNAcα1-3)Galβ1-3GalNAc
s) Fucα1-2Galβ1-4GlcNAcβ1-6GalNAc
t) Fucα1-2Galβ1-3 (GlcNAcβ1-6)GalNAc
u) Fucα1-2Galβ1-3 (6S-GlcNAcβ1-6)GalNAc
v) Fucα1-2Galβ1-3 (NeuAcβ2-6)GalNAc
w) GlcNAcβ1-3[Galβ1-4(6S)GlcNAcβ1-6]GalNAc
x) Galβ1-4GlcNAcβ1-3 [(6S)GlcNAcβ1-6]GalNAc
y) Galβ1-3(Fucα1-2Galβ1-4GlcNAcβ1-6)GalNAc
z) Fucα1-2Galβ1-4(6S)GlcNAcβ1-6[GlcNAcβ1-3]GalNAc
aa) GlcNAcβ1-3[Fucα1-2Galβ1-3(6S-)GlcNAcβ1-6]GalNAc
bb) Fucα1-2Gal+31-3 [Fucα1-2Galβ1-4(6 S)GlcNAcβ1-6]GalNAc.

24. The method according to claim 21, wherein the resulting composition comprising a mixture of glycopeptides comprises substantially no free glycans (w/w).

25. The method according to claim 21, wherein the obtained composition comprising a mixture of glycopeptides inhibits growth or reduces a level of Escherichia coli in the gut when orally administered to a subject more than a composition derived from the same process but not purified to remove insoluble particles greater than 7 μm.

26. A method for supplementing an animal feed comprising adding to the feed (1) between about 0.5% and 3.0% w/w of a composition comprising a mixture of glycopeptides obtained from gastrointestinal tract mucins, and (2) between about 97% and 99.5% of livestock feed, wherein:
a) the composition is obtained without subjecting the mucins or the partially purified fraction thereof to conditions or reagents that release oligosaccharides from glycopeptides;
b) the oligosaccharide content of the composition is >2% (w/w);
c) the peptide content of the composition, for peptides with a molecular weight of 5 KDa or less, is >40% (w/w);
d) the free amino acid content of the composition is <45% (w/w);
e) the water solubility of the mixture of glycopeptides is greater than 120 g/L at 25° C.;
f) the composition comprises glycopeptide-bound oligosaccharides having each of the following general formulae:
i. Hex₁HexNAc₁
ii. HexNAc₂
iii. NeuAc₁HexNAc₁
iv. NeuGc₁HexNAc₁
v. Hex₁HexNAc₁Fuc₁
vi. Hex₁HexNAc₂
vii. Hex₁HexNAc₂Sul₁
viii. NeuAc₁Hex₁HexNAc₁
ix. NeuGc₁Hex₁HexNAc₁
x. NeuAc₁HexNAc₂
xi. NeuGc₁HexNAc₂
xii. Hex₁HexNAc₂Fuc₁
xiii. Hex₁HexNAc₂Fuc₁Sul₁
xiv. NeuAc₁Hex₁HexNAc₁Fuc₁
xv. Hex₁HexNAc₃Sul₁
xvi. Hex₂HexNAc₂Fuc₁
xvii. Hex₁HexNAc₃Fuc₁Sul₁
xviii. Hex₂HexNAc₂Fuc₂Sul₁, and
g) the composition has been filtered to remove insoluble particles having a diameter greater than 7 μm.

27. The method according to claim 26, wherein the composition comprises glycopeptide-bound oligosaccharides having at least 7 of the structures shown in a) to bb):
a) Galβ1-3GalNAc
b) GlcNAcβ1-6GalNAc
c) NeuAcα2-6GalNAc
d) NeuGcα2-6GalNAc
e) Fucα1-2Galβ1-3GalNAc
f) Gal+GlcNAcβ1-6GalNAc
g) Galβ1-3(GlcNAcβ1-6)GalNAc
h) Galβ1-3 GlcNAcβ1-6GalNAc
i) Galβ1-3 (GlcNAcβ1-6)GalNAc
j) Galβ1-3 (6 SGlcNAcβ1-6)GalNAc
k) Galβ1-3 (NeuAcα2-6)GalNAc
l) NeuAcαα2-3 Galβ1-3 GalNAc
m) Galβ1-3 (NeuGcα2-6)GalNAc
n) NeuGcα2-3 Galβ1-3 GalNAc
o) GlcNAc-(NeuAcα2-6)GalNAc
p) GalNAc-(NeuAcα2-6)GalNAc
q) HexNAc-(NeuGcα2-6)GalNAc
r) Fucα1-2(GalNAcα1-3)Galβ1-3 GalNAc
s) Fucα1-2Galβ1-4GlcNAcβ1-6GalNAc
t) Fucα1-2Galβ1-3 (GlcNAcβ1-6)GalNAc
u) Fucα1-2Galβ1-3 (6 S-GlcNAcβ1-6)GalNAc
v) Fucα1-2Galβ1-3 (NeuAcβ2-6)GalNAc
w) GlcNAcβ1-3 [Galβ1-4(6 S)GlcNAcβ1-6]GalNAc
x) Galβ1-4GlcNAcβ1-3 [(6 S)GlcNAcβ1-6]GalNAc
y) Galβ1-3 (Fucα1-2Galβ1-4GlcNAcβ1-6)GalNAc
z) Fucα1-2Galβ1-4(6 S)GlcNAcβ1-6[GlcNAcβ1-3]GalNAc
aa) GlcNAcβ1-3 [Fucα1-2Galβ1-3 (6 S-)GlcNAcβ1-6]GalNAc
bb) Fucα1-2Galβ1-3 [Fucα1-2Galβ1-4(6 S)GlcNAcβ1-6]GalNAc.

28. The method according to claim 26, wherein the composition comprises ten sialylated glycopeptide-bound oligosaccharides selected from the following cc) through ll):
cc) NeuAcα2-6GalNAc
dd) NeuGcα2-6GalNAc
ee) Galβ1-3(NeuAcα2-6)GalNAc
ff) NeuAcαα2-3Galβ1-3GalNAc
gg) Galβ1-3(NeuGcα2-6)GalNAc
hh) NeuGcα2-3Galβ1-3GalNAc
ii) GlcNAc-(NeuAcα2-6)GalNAc
jj) GalNAc-(NeuAcα2-6)GalNAc
kk) HexNAc-(NeuGcα2-6)GalNAc; and
ll) Fucα1-2Galβ1-3(NeuAcβ2-6)GalNAc.

29. The method according to claim 26, having less than 0.1% (w/w) free glycans.

30. The method according to claim 26, wherein the gastrointestinal tract mucins are porcine gastrointestinal tract mucins.

31. The method of claim 26, wherein the commensal bacteria comprise Coprococcus comes, Prevotella copri, Megamonas spp., or Bacteroides vulgatus.

32. A method of increasing production of short chain fatty acid (SCFA) in the gut of a subject comprising orally administering to the subject a composition comprising a mixture of glycopeptides obtained from gastrointestinal tract mucins or a partially purified fraction thereof, wherein:
a) the composition is obtained without subjecting the mucins or the partially purified fraction thereof to conditions or reagents that release oligosaccharides from glycopeptides;
b) the oligosaccharide content of the composition is >2% (w/w);
c) the peptide content of the composition, for peptides with a molecular weight of 5 KDa or less, is >40% (w/w);
d) the free amino acid content of the composition is <45% (w/w);
e) the water solubility of the mixture of glycopeptides is greater than 120 g/L at 25° C.;
f) the composition comprises glycopeptide-bound oligosaccharides having each of the following general formulae:
i. Hex₁HexNAc₁
ii. HexNAc₂
iii. NeuAc₁HexNAc₁
iv. NeuGc₁HexNAc₁
v. Hex₁HexNAc₁Fuc₁
vi. Hex₁HexNAc₂
vii. Hex₁HexNAc₂Sul₁
viii. NeuAc₁Hex₁HexNAc₁
ix. NeuGc₁Hex₁HexNAc₁
x. NeuAc₁HexNAc₂
xi. NeuGc₁HexNAc₂
xii. Hex₁HexNAc₂Fuc₁
xiii. Hex₁HexNAc₂Fuc₁Sul₁
xiv. NeuAc₁Hex₁HexNAc₁Fuc₁
xv. Hex₁HexNAc₃Sul₁
xvi. Hex₂HexNAc₂Fuc₁
xvii. Hex₁HexNAc₃Fuc₁Sul₁
xviii. Hex₂HexNAc₂Fuc₂Sul₁, and
g) the composition has been filtered to remove insoluble particles having a diameter greater than 7 μm.

33. The method according to claim 32, wherein the composition comprises glycopeptide-bound oligosaccharides having at least 7 of the structures shown in a) to bb):
a) Galβ1-3GalNAc
b) GlcNAcβ1-6GalNAc
c) NeuAcα2-6GalNAc
d) NeuGcα2-6GalNAc
e) Fucα1-2Galβ1-3GalNAc
f) Gal+GlcNAcβ1-6GalNAc
g) Galβ1-3 (GlcNAcβ1-6)GalNAc
h) Galβ1-3 GlcNAcβ1-6GalNAc
i) Galβ1-3 (GlcNAcβ1-6)GalNAc
j) Galβ1-3(6 SGlcNAcβ1-6)GalNAc
k) Galβ1-3(NeuAcα2-6)GalNAc
l) NeuAcαα2-3 Galβ1-3 GalNAc
m) Galβ1-3(NeuGcα2-6)GalNAc
n) NeuGcα2-3 Galβ1-3 GalNAc
o) GlcNAc-(NeuAcα2-6)GalNAc
p) GalNAc-(NeuAcα2-6)GalNAc
q) HexNAc-(NeuGcα2-6)GalNAc
r) Fucα1-2(GalNAcα1-3)Galβ1-3GalNAc
s) Fucα1-2Galβ1-4GlcNAcβ1-6GalNAc
t) Fucα1-2Galβ1-3(GlcNAcβ1-6)GalNAc
u) Fucα1-2Galβ1-3 (6 S-GlcNAcβ1-6)GalNAc
v) Fucα1-2Galβ1-3(NeuAcβ2-6)GalNAc
w) GlcNAcβ1-3 [Galβ1-4(6S)GlcNAcβ1-6]GalNAc
x) Galβ1-4GlcNAcβ1-3 [(6 S)GlcNAcβ1-6]GalNAc
y) Galβ1-3(Fucα1-2Galβ1-4GlcNAcβ1-6)GalNAc
z) Fucα1-2Galβ1-4(6 S)GlcNAcβ1-6 [GlcNAcβ1-3]GalNAc
aa) GlcNAcβ1-3 [Fucα1-2Galβ1-3 (6 S-)GlcNAcβ1-6]GalNAc
bb) Fucα1-2Galβ1-3 [Fucα1-2Galβ1-4(6 S)GlcNAcβ1-6]GalNAc.

34. The method according to claim 32, wherein the composition comprises ten sialylated glycopeptide-bound oligosaccharides selected from the following cc) through ll):
cc) NeuAcα2-6GalNAc
dd) NeuGcα2-6GalNAc
ee) Galβ1-3(NeuAcα2-6)GalNAc
ff) NeuAcαα2-3Galβ1-3GalNAc
gg) Galβ1-3(NeuGcα2-6)GalNAc
hh) NeuGcα2-3Galβ1-3GalNAc
ii) GlcNAc-(NeuAcα2-6)GalNAc
jj) GalNAc-(NeuAcα2-6)GalNAc
kk) HexNAc-(NeuGcα2-6)GalNAc; and
ll) Fucα1-2Galβ1-3(NeuAcβ2-6)GalNAc.

35. The method according to claim 32, having less than 0.1% (w/w) free glycans.

36. The method according to claim 32, wherein the gastrointestinal tract mucins are porcine gastrointestinal tract mucins.

37. The method of claim 32, wherein the commensal bacteria comprise Coprococcus comes, Prevotella copri, Megamonas spp., or Bacteroides vulgatus.

38. The method of claim 32, wherein the pH in the gut of the subject is decreased.
